SEQUENCE LISTING (13 sequences)

SEQ ID NO: 1 - DNA, 66 bp, Artificial Sequence
  CS-seq primer PCR1-Fs
gatctacactctttccctacacgacgctcttccgatctgctagcactgtacctaggactgagctag

SEQ ID NO: 2 - DNA, 95 bp, Artificial Sequence
  CS-seq primer PCR2-F
aatgatacggcgaccaccgagatctacacccatgttggctcattggaaaccactacagatctacactctttccctacacgacgctcttccgatct

SEQ ID NO: 3 - DNA, 21 bp, Artificial Sequence
  CS-seq primer PCR1-R
gtctcgtgggctcggagatgt

SEQ ID NO: 4 - DNA, 67 bp, Artificial Sequence
  CS-seq primer PCR2-R
caagcagaagacggcatacgagatgctgtgttgatcataggctccgagtcttgtctcgtgggctcgg

SEQ ID NO: 5 - DNA, 37 bp, Artificial Sequence
  Primer SG-seq PCR1-F
tcgtcggcagcgtctatttacctcctttatgctagca

SEQ ID NO: 6 - DNA, 71 bp, Artificial Sequence
  Primer SG-seq PCR2-F
aatgatacggcgaccaccgagatctacacccatgttggctcattggaaaccactacatcgtcggcagcgtc

SEQ ID NO: 7 - DNA, 30 bp, Artificial Sequence
  Primer SG-seq PCR1-R
  misc_feature (16)..(25): n is a, c, g, or t
gtctcgtgggctcggnnnnnnnnnntgcgg

SEQ ID NO: 8 - DNA, 30 bp, Artificial Sequence
  Primer SG-seq PCR1-R
  misc_feature (16)..(26): n is a, c, g, or t
gtctcgtgggctcggnnnnnnnnnnngcgg

SEQ ID NO: 9 - DNA, 30 bp, Artificial Sequence
  Primer SG-seq PCR1-R
  misc_feature (16)..(27): n is a, c, g, or t
gtctcgtgggctcggnnnnnnnnnnnncgg

SEQ ID NO: 10 - DNA, 30 bp, Artificial Sequence
  Primer SG-seq PCR1-R
  misc_feature (16)..(25): n is a, c, g, or t
gtctcgtgggctcggnnnnnnnnnnctata

SEQ ID NO: 11 - DNA, 30 bp, Artificial Sequence
  Primer SG-seq PCR1-R
  misc_feature (16)..(26): n is a, c, g, or t
gtctcgtgggctcggnnnnnnnnnnntata

SEQ ID NO: 12 - DNA, 30 bp, Artificial Sequence
  Primer SG-seq PCR1-R
  misc_feature (16)..(27): n is a, c, g, or t
gtctcgtgggctcggnnnnnnnnnnnnata

SEQ ID NO: 13 - DNA, 67 bp, Artificial Sequence; identical to SEQ ID NO: 4
  Primer SG-seq PCR2-R

(19) United States
(12) Patent Application Publication
(10) Pub. No.: US 2021/0115500 A1
(43) Pub. Date: Apr. 22, 2021

(54) GENOTYPING EDITED MICROBIAL STRAINS

(71) Applicant: Zymergen Inc., Emeryville, CA (US)

(72) Inventors: Philip D. WEYMAN, Alameda, CA (US); Kedar PATEL, Fremont, CA (US); Aaron MILLER, El Cerrito, CA (US); Sara da Luz Areosa CLETO, Emeryville, CA (US); Kunal MEHTA, Oakland, CA (US)

(21) Appl. No.: 17/072,449

(22) Filed: Oct. 16, 2020

Related U.S. Application Data
(60) Provisional application No. 62/923,355, filed on Oct. 18, 2019.

(51) Int. Cl. C12Q 1/689; G16B 30/10; G16B 20/00

(52) U.S. Cl. CPC C12Q 1/689; G16B 20/00; G16B 30/10

(57) ABSTRACT

The present invention relates to methods for genotyping microbial host cells that have been subjected to metabolic engineering. The methods provided herein allow detection of genetic edits in the genome of a microbial host cell using PCR-based genome enrichment following appendage of a common priming site. The compositions and methods of the present invention can be used to confirm engineered metabolic diversity as well as ectopic insertions. Kits for performing the methods are also disclosed.

### CROSS-REFERENCE TO RELATED APPLICATIONS

This application claims the benefit of priority to U.S. Provisional Application Ser. No. 62/923,355, filed Oct. 18, 2019, which is herein incorporated by reference in its entirety for all purposes.

### FIELD

The present disclosure is directed to compositions and methods for genotyping microbial strains whose genomes have been edited. The disclosed methods and compositions can be useful for determining and/or confirming the location of a genetic edit or each of a plurality of genetic edits introduced into the genome of a desired host cell or organism. Further, the compositions and methods provided herein can be useful for identifying and tracking engineered diversity as opposed to natural or random diversity

### STATEMENT REGARDING SEQUENCE LISTING

The Sequence Listing associated with this application is provided in text format in lieu of a paper copy, and is hereby incorporated by reference into the specification. The name of the text file containing the Sequence Listing is ZYMR-043_01US_SeqList_ST25.txt. The text file is about 3.47 KB, and was created on Oct. 16, 2020, and is being submitted electronically via EFS-Web.

### BACKGROUND

Metabolic engineering is widely applied to modify microbial host cells such as Escherichia coli to produce industrially relevant biofuels or biochemicals, including ethanol, higher alcohols, fatty acids, amino acids, shikimate precursors, terpenoids, polyketides, and polymeric precursors of 1,4-butanediol. Often, industrially optimized strains require numerous genomic modifications, including insertions, deletions, and regulatory modifications in order to produce such industrially relevant products. Such large numbers of genome editing targets require efficient tools to perform time-saving sequential manipulations or multiplex manipulations as well as to determine and/or confirm that each designed genetic manipulation occurred in the proper location within the genome of the host cell or organism. Genotyping of microbial strains subjected to metabolic engineering techniques is typically performed by whole genome sequencing (WGS) techniques or polymerase chain reaction (PCR) of the target genetic manipulations followed by cloning and sequencing. Either of these techniques can be useful when an organism contains a single or small number of possible genetic manipulations. However, the use of PCR of the target genetic manipulations followed by cloning and sequencing is impractical when the metabolic engineering is performed using a library or pooled approach where the resultant organisms could contain one of many possible edits. Moreover, use of WGS to identify genetic manipulations is expensive, data and computation intensive and capacity limited when screening thousands of colonies for metabolic engineering experiments performed in a high-throughput fashion. Furthermore, because WGS is negatively impacted by genome size, WGS solutions might not scale as easily, especially when the organism subjected to the high-throughput metabolic engineering has a genome that is quite large.

Thus, there is a need in the art for new methods for determining and/or confirming the genomic locations of genetic edits introduced into microbial host cells in an efficient, rapid, accurate and cost-effective manner that can be utilized across multiple strains in a high-throughput manner. The compositions and methods provided herein address the aforementioned drawbacks inherent with current methods for genotyping engineered or ectopic metabolic diversity in microbial host cells.

### SUMMARY

In one aspect, provided herein is a method for identifying one or a plurality of genetic edits introduced into a microbial strain, the method comprising: (a) appending an adaptor comprising a universal sequence to nucleic acid fragments from a plurality of nucleic acid fragments prepared from nucleic acid obtained or derived from a microbial strain, wherein the microbial strain comprises the one or the plurality of genetic edits, wherein each genetic edit from the one or the plurality of genetic edits comprises a common sequence; (b) amplifying each of the nucleic acid fragments from step (a) in a polymerase chain reaction (PCR) using a primer pair comprising a first primer comprising a sequence complementary to the common sequence at its 3′ end and a 5′ tail comprising non-complementary sequence and a second primer comprising sequence complementary to the universal sequence at its 3′ end and a 5′ tail comprising non-complementary sequence, optionally, wherein the non-complementary sequence of the first primer and the second primer each comprise sequencing primer binding sites; and (c) performing molecular analysis on amplicons generated from the PCR performed in step (b), thereby identifying the one or the plurality of genetic edits in the microbial strain. In some cases, step (a) is performed in a transposon mediated adapter addition reaction. In some cases, step (a) is performed in a tagmentation reaction. In some cases, step (a) is performed by fragmenting the nucleic acid obtained or derived from the microbial strain and ligating the adaptors comprising the universal sequence to the nucleic acid fragments. In some cases, the non-complementary sequence of the first primer and/or the second primer further comprise a sample specific index sequence. In some cases, the molecular analysis comprises amplicon size selection on the amplicons generated from the PCR performed in step (b). In some cases, the amplicon size selection comprises digestion and/or gel electrophoresis of the amplicons, optionally wherein the electrophoresis is preceded by the digestion. In some cases, the first primer is specific to a genetic edit and the second primer is specific to a single universal sequence found in each adapter. In some cases, the molecular analysis comprises DNA sequencing. In some cases, the molecular analysis of the amplicons comprises DNA sequencing using sequencing primers directed to the sequencing primer binding sites. In some cases, the molecular analysis comprises first, second, or third generation DNA sequencing. In some cases, the method further comprises comparing sequence reads obtained from the sequencing of the amplicons to a reference database for the microbial strain using a computer-implemented method, thereby identifying the one or the plurality of genetic edits. In some cases, the computer-implemented method utilizes a sequence similarity search program, a sequence composition search program or a combination thereof. In some cases, the sequence similarity search program employs a basic local alignment search tool (BLAST) algorithm, fuzzy logic, lowest common ancestor (LCA) algorithm or a profile hidden Markov Model (pHMM). In some cases, the sequence composition search program employs interpolated Markov models (IMMs), naive Bayesian classifiers, k-mers or k-means/k-nearest-neighbor algorithms. In some cases, the sequence composition search program employs k-mers. In some cases, the k-mers comprise short nucleotide sequences comprising nucleotide bases complementary to a sequence near the one or each of the plurality of genetic edits, wherein detection of the short nucleotide sequence in the sequence reads indicates presence of the one or each of the plurality of genetic edits in the microbial strain. In some cases, the sequence near the one or each of the plurality of genetic edits is within 25 base pairs (bps), 20 bps, 15 bps, 10 bps, or 5 bps of the one or each of the plurality of genetic edits. In some cases, the one or the plurality of genetic edits is in an episome, chromosome, or other genomic DNA. In some cases, the chromosome is from bacteria or fungi. In some cases, the obtaining or derivation of the nucleic acid entails lysing the microbial strain. In some cases, the obtaining or derivation of the nucleic acid entails isolating the nucleic acid from the microbial strain. In some cases, the obtaining or derivation of the nucleic acid entails whole genome amplification (WGA) or multiple displacement amplification (MDA) of nucleic acid isolated from the microbial strain. In some cases, the obtaining or derivation of the nucleic acid entails performing a boil preparation of the microbial strain. In some cases, the common sequence in at least one genetic edit in the plurality of genetic edits is different from the common sequence in each other genetic edit in the plurality of genetic edits. In some cases, the common sequence in each genetic edit in the plurality of genetic edits is different from the common sequence in each other genetic edit in the plurality of genetic edits. In some cases, the common sequence is selected from any genetic element including a promoter sequence, a termination sequence, a degron sequence, a protein solubility tag sequence, a protein degradation tag sequence, a ribosomal binding site (RBS) sequence, a landing pad primer binding sequence, an antibiotic resistance gene sequence or any portion thereof. In some cases, the common sequence is specific to a genetic edit.

In another aspect, provided herein is a method for identifying one or a plurality of genetic edits introduced into a microbial strain, the method comprising: (a) appending an adaptor comprising a universal sequence to nucleic acid fragments from a plurality of nucleic acid fragments prepared from nucleic acid obtained or derived from a microbial strain, wherein the microbial strain comprises the one or the plurality of genetic edits, wherein each genetic edit from the one or the plurality of genetic edits comprises a common sequence; (b) amplifying each of the nucleic acid fragments from step (a) in a first polymerase chain reaction (PCR) using a first primer pair comprising a first primer comprising a sequence complementary to the common sequence at its 3′ end and a 5′ tail comprising non-complementary sequence and a second primer comprising sequence complementary to the universal sequence at its 3′ end and a 5′ tail comprising non-complementary sequence; (c) amplifying amplicons generated in step (b) in a second PCR using a second primer pair comprising a first primer comprising a 3′ end comprising sequence complementary to the non-complementary sequence in the 5′ tail of the first primer from the first primer pair and a second primer comprising a 3′ end comprising sequence complementary to the non-complementary sequence in the 5′ tail of the second primer from the first primer pair, wherein the first primer and the second primer from the second primer pair each comprise 5′ tails comprising non-complementary sequence and, optionally each of the 5′ tails comprising non-complementary sequence from the second primer pair comprise sequencing primer binding sites; and (d) performing molecular analysis on amplicons generated from the PCR performed in step (c), thereby identifying the one or the plurality of genetic edits in the microbial strain. In some cases, step (a) is performed in a transposon mediated adapter addition reaction. In some cases, step (a) is performed in a tagmentation reaction. In some cases, step (a) is performed by fragmenting the nucleic acid obtained or derived from the microbial strain and ligating the adaptors comprising the universal sequence to the nucleic acid fragments. In some cases, the non-complementary sequence of the first primer and/or the second primer of the second primer pair further comprise a sample specific index sequence. In some cases, the molecular analysis comprises amplicon size selection on the amplicons generated from the PCR performed in step (c). In some cases, the amplicon size selection comprises digestion and/or gel electrophoresis of the amplicons, optionally wherein the electrophoresis is preceded by the digestion. In some cases, the first primer of the second primer pair is specific to a genetic edit and the second primer of the second primer pair is specific to a single universal sequence found in each adapter. In some cases, the molecular analysis comprises DNA sequencing. In some cases, the molecular analysis of the amplicons comprises DNA sequencing using sequencing primers directed to the sequencing primer binding sites. In some cases, the molecular analysis comprises first, second, or third generation DNA sequencing. In some cases, the method further comprises comparing sequence reads obtained from the sequencing of the amplicons to a reference database for the microbial strain using a computer-implemented method, thereby identifying the one or the plurality of genetic edits. In some cases, the computer-implemented method utilizes a sequence similarity search program, a sequence composition search program or a combination thereof. In some cases, the sequence similarity search program employs a basic local alignment search tool (BLAST) algorithm, fuzzy logic, lowest common ancestor (LCA) algorithm or a profile hidden Markov Model (pHMM). In some cases, the sequence composition search program employs interpolated Markov models (IMMs), naive Bayesian classifiers, k-mers or k-means/k-nearest-neighbor algorithms. In some cases, the sequence composition search program employs k-mers. In some cases, the k-mers comprise short nucleotide sequences comprising nucleotide bases complementary to a sequence near the one or each of the plurality of genetic edits, wherein detection of the short nucleotide sequence in the sequence reads indicates presence of the one or each of the plurality of genetic edits in the microbial strain. In some cases, the sequence near the one or each of the plurality of genetic edits is within 25 base pairs (bps), 20 bps, 15 bps, 10 bps, or 5 bps of the one or each of the plurality of genetic edits. In some cases, the one or the plurality of genetic edits is in an episome, chromosome, or other genomic DNA. In some cases, the chromosome is from bacteria or fungi. In some cases, the obtaining or derivation of the nucleic acid entails lysing the microbial strain. In some cases, the obtaining or derivation of the nucleic acid entails isolating the nucleic acid from the microbial strain. In some cases, the obtaining or derivation of the nucleic acid entails whole genome amplification (WGA) or multiple displacement amplification (MDA) of nucleic acid isolated from the microbial strain. In some cases, the obtaining or derivation of the nucleic acid entails performing a boil preparation of the microbial strain. In some cases, the common sequence in at least one genetic edit in the plurality of genetic edits is different from the common sequence in each other genetic edit in the plurality of genetic edits. In some cases, the common sequence in each genetic edit in the plurality of genetic edits is different from the common sequence in each other genetic edit in the plurality of genetic edits. In some cases, the common sequence is selected from any genetic element including a promoter sequence, a termination sequence, a degron sequence, a protein solubility tag sequence, a protein degradation tag sequence, a ribosomal binding site (RBS) sequence, a landing pad primer binding sequence, an antibiotic resistance gene sequence or any portion thereof. In some cases, the common sequence is specific to a genetic edit.

In yet another aspect, provided herein is a method for identifying one or a plurality of genetic edits introduced into a microbial strain, the method comprising: (a) amplifying nucleic acid obtained or derived from a microbial strain in a first polymerase chain reaction (PCR), wherein the microbial strain comprises the one or the plurality of genetic edits, and wherein each genetic edit from the one or the plurality of genetic edits comprises a common sequence, wherein the first PCR utilizes a first primer pair comprising a first primer comprising a sequence complementary to the common sequence at its 3′ end and a 5′ tail comprising a first universal sequence and a plurality of second primers comprising a priming sequence complementary to a variable locus-specific sequence at its 3′ end and a 5′ tail comprising a second universal sequence that is common among all second primers; (b) amplifying amplicons generated in step (a) in a second PCR using a second primer pair comprising a first primer comprising a 3′ end comprising sequence complementary to the first universal sequence in the 5′ tail of the first primer from the first primer pair and a second primer comprising a 3′ end comprising sequence complementary to the second universal sequence in the 5′ tail of each of the second primers from the first primer pair, wherein the first primer and the second primer from the second primer pair each comprise 5′ tails comprising non-complementary sequence and, optionally each of the 5′ tails comprising non-complementary sequence from the second primer pair; and (c) performing molecular analysis on amplicons generated from the second PCR performed in step (b), thereby identifying the one or the plurality of genetic edits in the microbial strain. In some cases, the non-complementary sequence of the first primer and/or the second primer of the second primer pair further comprise a sample specific index sequence. In some cases, the priming sequence in the plurality of second primers comprises a mixture of fully or partially random nucleotides and nucleotides that are complementary to the variable locus-specific sequence. In some cases, the priming sequence comprises at least 1, at least 2, at least 3, at least 4, at least 5, at least 6, at least 7, at least 8, at least 9 or at least 10 nucleotides that are complementary to the variable locus-specific sequence. In some cases, the priming sequence comprises at least 3-5 nucleotides that are complementary to the variable locus-specific sequence. In some cases, the priming sequence comprises 1, 2, 3, 4, 5, 6, 7, 8, 9 or 10 nucleotides that are complementary to the variable locus-specific sequence. In some cases, the priming sequence comprises between 0-3, between 1-4, between 2-5, between 3-6, between 4-7, between 5-8, between 6-9, between 7-10 or between 8-11 nucleotides that are complementary to the variable locus-specific sequence. In some cases, the variable locus-specific sequence is near the one or each genetic edit from the plurality of genetic edits. In some cases, the variable locus-specific sequence is present in the microbial strain at least once near the one or each genetic edit of the plurality of genetic edits. In some cases, the variable locus-specific sequence is less than 3 kilobases (kbs), less than 1.5 kbs, less than 1 kb, less than 750 base-pairs (bps), less than 500 bps, less than 250 bps, less than 125 bps, less than 100 bps, less than 75 bps, less than 50 bps, less than 25 bps, less than 20 bps, less than 15 bps, less than 10 bps, or less than 5 bps away from the one or each of the plurality of genetic edits. In some cases, the variable locus-specific sequence is less than 1.5 kb away from the one or each of the plurality of genetic edits. In some cases, the molecular analysis comprises amplicon size selection on the amplicons generated from the PCR performed in step (b). In some cases, the amplicon size selection comprises digestion and/or gel electrophoresis of the amplicons, optionally wherein the electrophoresis is preceded by the digestion. In some cases, the molecular analysis comprises DNA sequencing. In some cases, the molecular analysis of the amplicons comprises DNA sequencing using sequencing primers directed to the sequencing primer binding sites. In some cases, the molecular analysis comprises first, second, or third generation DNA sequencing. In some cases, the method further comprises comparing sequence reads obtained from the sequencing of the amplicons to a reference database for the microbial strain using a computer-implemented method, thereby identifying the one or the plurality of genetic edits. In some cases, the computer-implemented method utilizes a sequence similarity search program, a sequence composition search program or a combination thereof. In some cases, the sequence similarity search program employs a basic local alignment search tool (BLAST) algorithm, fuzzy logic, lowest common ancestor (LCA) algorithm or a profile hidden Markov Model (pHMM). In some cases, the sequence composition search program employs interpolated Markov models (IMMs), naive Bayesian classifiers, k-mers or k-means/k-nearest-neighbor algorithms. In some cases, the sequence composition search program employs k-mers. In some cases, the k-mers comprise short nucleotide sequences comprising nucleotide bases complementary to a sequence near the one or each of the plurality of genetic edits, wherein detection of the short nucleotide sequence in the sequence reads indicates presence of the one or each of the plurality of genetic edits in the microbial strain. In some cases, the sequence near the one or each of the plurality of genetic edits is within 25 base pairs (bps), 20 bps, 15 bps, 10 bps, or 5 bps of the one or each of the plurality of genetic edits. In some cases, the one or the plurality of genetic edits is in an episome, chromosome, or other genomic DNA. In some cases, the chromosome is from bacteria or fungi. In some cases, the obtaining or derivation of the nucleic acid entails lysing the microbial strain. In some cases, the derivation of the nucleic acid entails isolating the nucleic acid from the microbial strain. In some cases, the obtaining or derivation of the nucleic acid entails whole genome amplification (WGA) or multiple displacement amplification (MDA) of nucleic acid isolated from the microbial strain. In some cases, the obtaining or derivation of the nucleic acid entails performing a boil preparation of the microbial strain. In some cases, the common sequence in at least one genetic edit in the plurality of genetic edits is different from the common sequence in each other genetic edit in the plurality of genetic edits. In some cases, the common sequence in each genetic edit in the plurality of genetic edits is different from the common sequence in each other genetic edit in the plurality of genetic edits. In some cases, the common sequence is selected from any genetic element including a promoter sequence, a termination sequence, a degron sequence, a protein solubility tag sequence, a protein degradation tag sequence, a ribosomal binding site (RBS) sequence, a landing pad primer binding sequence, an antibiotic resistance gene sequence or any portion thereof. In some cases, the common sequence is specific to a genetic edit.

In still another aspect, provided herein is a method for identifying one or a plurality of genetic edits introduced into a microbial strain, the method comprising: (a) amplifying nucleic acid obtained or derived from a microbial strain in a polymerase chain reaction (PCR), wherein the microbial strain comprises the one or the plurality of genetic edits, and wherein each genetic edit from the one or the plurality of genetic edits comprises a common sequence, wherein the PCR utilizes a primer pair comprising a first primer comprising a sequence complementary to the common sequence at its 3′ end and a 5′ tail comprising a first universal sequence and a plurality of second primers comprising a priming sequence complementary to a variable locus-specific sequence at its 3′ end and a 5′ tail comprising a second universal sequence that is common among all second primers, optionally, wherein the first primer and each second primer of the plurality of second primers each comprise sequencing primer binding sites in the 5′ tail; and (b) performing molecular analysis on amplicons generated from the PCR performed in step (a), thereby identifying the one or the plurality of genetic edits in the microbial strain. In some cases, the non-complementary sequence of the first primer and/or the second primer further comprise a sample specific index sequence. In some cases, the priming sequence in the plurality of second primers comprises a mixture of fully or partially random nucleotides and nucleotides that are complementary to the variable locus-specific sequence. In some cases, the priming sequence comprises at least 1, at least 2, at least 3, at least 4, at least 5, at least 6, at least 7, at least 8, at least 9 or at least 10 nucleotides that are complementary to the variable locus-specific sequence. In some cases, the priming sequence comprises at least 3-5 nucleotides that are complementary to the variable locus-specific sequence. In some cases, the priming sequence comprises 1, 2, 3, 4, 5, 6, 7, 8, 9 or 10 nucleotides that are complementary to the variable locus-specific sequence. In some cases, the priming sequence comprises between 0-3, between 1-4, between 2-5, between 3-6, between 4-7, between 5-8, between 6-9, between 7-10 or between 8-11 nucleotides that are complementary to the variable locus-specific sequence. In some cases, the variable locus-specific sequence is near the one or each genetic edit from the plurality of genetic edits. In some cases, the variable locus-specific sequence is present in the microbial strain at least once near the one or each genetic edit of the plurality of genetic edits. In some cases, the variable locus-specific sequence is less than 3 kilobases (kbs), less than 1.5 kbs, less than 1 kb, less than 750 base-pairs (bps), less than 500 bps, less than 250 bps, less than 125 bps, less than 100 bps, less than 75 bps, less than 50 bps, less than 25 bps, less than 20 bps, less than 15 bps, less than 10 bps, or less than 5 bps away from the one or each of the plurality of genetic edits. In some cases, the variable locus-specific sequence is less than 1.5 kb away from the one or each of the plurality of genetic edits. In some cases, the molecular analysis comprises amplicon size selection on the amplicons generated from the PCR performed in step (a). In some cases, the amplicon size selection comprises digestion and/or gel electrophoresis of the amplicons, optionally wherein the electrophoresis is preceded by the digestion. In some cases, the molecular analysis comprises DNA sequencing. In some cases, the molecular analysis of the amplicons comprises DNA sequencing using sequencing primers directed to the sequencing primer binding sites. In some cases, the molecular analysis comprises first, second, or third generation DNA sequencing. In some cases, the method further comprises comparing sequence reads obtained from the sequencing of the amplicons to a reference database for the microbial strain using a computer-implemented method, thereby identifying the one or the plurality of genetic edits. In some cases, the computer-implemented method utilizes a sequence similarity search program, a sequence composition search program or a combination thereof. In some cases, the sequence similarity search program employs a basic local alignment search tool (BLAST) algorithm, fuzzy logic, lowest common ancestor (LCA) algorithm or a profile hidden Markov Model (pHMM). In some cases, the sequence composition search program employs interpolated Markov models (IMMs), naive Bayesian classifiers, k-mers or k-means/k-nearest-neighbor algorithms. In some cases, the sequence composition search program employs k-mers. In some cases, the k-mers comprise short nucleotide sequences comprising nucleotide bases complementary to a sequence near the one or each of the plurality of genetic edits, wherein detection of the short nucleotide sequence in the sequence reads indicates presence of the one or each of the plurality of genetic edits in the microbial strain. In some cases, the sequence near the one or each of the plurality of genetic edits is within 25 base pairs (bps), 20 bps, 15 bps, 10 bps, or 5 bps of the one or each of the plurality of genetic edits. In some cases, the one or the plurality of genetic edits is in an episome, chromosome, or other genomic DNA. In some cases, the chromosome is from bacteria or fungi. In some cases, the obtaining or derivation of the nucleic acid entails lysing the microbial strain. In some cases, the obtaining or derivation of the nucleic acid entails isolating the nucleic acid from the microbial strain. In some cases, the obtaining or derivation of the nucleic acid entails whole genome amplification (WGA) or multiple displacement amplification (MDA) of nucleic acid isolated from the microbial strain. In some cases, the obtaining or derivation of the nucleic acid entails performing a boil preparation of the microbial strain. In some cases, the common sequence in at least one genetic edit in the plurality of genetic edits is different from the common sequence in each other genetic edit in the plurality of genetic edits. In some cases, the common sequence in each genetic edit in the plurality of genetic edits is different from the common sequence in each other genetic edit in the plurality of genetic edits. In some cases, the common sequence is selected from any genetic element including a promoter sequence, a termination sequence, a degron sequence, a protein solubility tag sequence, a protein degradation tag sequence, a ribosomal binding site (RBS) sequence, a landing pad primer binding sequence, an antibiotic resistance gene sequence or any portion thereof. In some cases, the common sequence is specific to a genetic edit.

In some cases, the genetic edits were introduced into the microbial strain by an iterative editing method, wherein the iterative method comprises: (a) introducing into a microbial host cell a first plasmid comprising a first repair fragment and a selection marker gene, wherein the microbial host cell comprises a site-specific restriction enzyme or a sequence encoding a site-specific restriction enzyme is introduced into the microbial host cell along with the first plasmid, wherein the site-specific restriction enzyme targets a first locus in the microbial host cell, and wherein the first repair fragment comprises homology arms separated by a sequence for a genetic edit comprising a common sequence in or adjacent to a first locus in the microbial host cell, wherein the homology arms comprise sequence homologous to sequence that flanks the first locus in the microbial host cell; (b) growing the microbial host cells from step (a) in a medium selective for microbial host cells expressing the selection marker gene and isolating microbial host cells from cultures derived therefrom; (c) growing the microbial host cells isolated in step (b) in media not selective for the selection marker gene and isolating microbial host cells from cultures derived therefrom; and (d) repeating steps (a)-(c) in one or more additional rounds in the microbial host cells isolated in step (c), wherein each of the one or more additional rounds comprises introducing an additional plasmid comprising an additional repair fragment, wherein the additional repair fragment comprises homology arms separated by a sequence for a genetic edit comprising a common sequence in or adjacent to a locus in the microbial host cell, wherein the homology arms comprise sequence homologous to sequence that flanks the locus in the microbial host cell, wherein the additional plasmid comprises a different selection marker gene than the selection marker gene introduced in a previous round of selection, and wherein the microbial host cell comprises a site-specific restriction enzyme or a sequence encoding a site-specific restriction enzyme is introduced into the microbial host cell along with the additional plasmid that targets the first locus or another locus in the microbial host cell, thereby iteratively editing the microbial host cell to generate the microbial strain comprising the plurality of genetic edits; wherein a counterselection is not performed after at least one round of editing.

In some cases, the genetic edits were introduced into the microbial strain by an iterative editing method, wherein the iterative method comprises: (a) introducing into the microbial host cell a first plasmid, a first guide RNA (gRNA) and a first repair fragment, wherein the gRNA comprises a sequence complementary to a first locus in the microbial host cell, wherein the first repair fragment comprises homology arms separated by a sequence for a genetic edit comprising a common sequence in or adjacent to a first locus in the microbial host cell, wherein the homology arms comprise sequence homologous to sequence that flanks the first locus in the microbial host cell, wherein the first plasmid comprises a selection marker gene and at least one or both of the gRNA and the repair fragment, and wherein: (i) the microbial host cell comprises an RNA-guided DNA endonuclease; or (ii) an RNA-guided DNA endonuclease is introduced into the microbial host cell along with the first plasmid; (b) growing the microbial host cells from step (a) in a medium selective for microbial host cells expressing the selection marker gene and isolating microbial host cells from cultures derived therefrom; (c) growing the microbial host cells isolated in step (b) in media not selective for the selection marker gene and isolating microbial host cells from cultures derived therefrom; and (d) repeating steps (a)-(c) in one or more additional rounds in the microbial host cells isolated in step (c), wherein each of the one or more additional rounds comprises introducing an additional plasmid, an additional gRNA and an additional repair fragment, wherein the additional gRNA comprises sequence complementary to a locus in the microbial host cell, wherein the additional repair fragment homology arms separated by a sequence for a genetic edit comprising a common sequence in or adjacent to a locus in the microbial host cell, wherein the homology arms comprise sequence homologous to sequence that flanks the locus in the microbial host cell, wherein the additional plasmid comprises a different selection marker gene than the selection marker gene introduced in a previous round of selection, and wherein the additional plasmid comprises at least one or both of the additional gRNA and the additional repair fragment, thereby iteratively editing the microbial host cell to generate the microbial strain comprising the plurality of genetic edits; wherein a counterselection is not performed after at least one round of editing.

In some cases, the genetic edits were introduced into the microbial strain by an iterative editing method, wherein the iterative method comprises: (a) introducing into the microbial host cell a first plasmid comprising a first repair fragment and a selection marker gene, wherein the first repair fragment comprises homology arms separated by a sequence for a genetic edit comprising a common sequence in or adjacent to a first locus in the microbial host cell, wherein the homology arms comprise sequence homologous to sequence that flanks the first locus in the microbial host cell; (b) growing the microbial host cells from step (a) in a medium selective for microbial host cells expressing the selection marker gene and isolating microbial host cells from cultures derived therefrom; (c) growing the microbial host cells isolated in step (b) in media not selective for the selection marker gene and isolating microbial host cells from cultures derived therefrom; and (d) repeating steps (a)-(c) in one or more additional rounds in the microbial host cells isolated in step (c), wherein each of the one or more additional rounds comprises introducing an additional plasmid comprising an additional repair fragment, wherein the additional repair fragment comprises homology arms separated by sequence for a genetic edit comprising a common sequence in or adjacent to a locus in the microbial host cell, wherein the homology arms comprise sequence homologous to sequence that flanks the locus in the microbial host cell, and wherein the additional plasmid comprises a different selection marker gene than the selection marker gene introduced in a previous round of selection, thereby iteratively editing the microbial host cell to generate the microbial strain comprising the plurality of genetic edits; wherein a counterselection is not performed after at least one round of editing.

In some cases, the genetic edits were introduced into the microbial strain by a pooled editing method, wherein the pooled method comprises: (a) combining a base population of microbial host cells with a first pool of editing plasmids, wherein each editing plasmid in the pool comprises at least one repair fragment, and wherein the pool of editing plasmids comprises at least two different repair fragments, wherein each editing plasmid in the pool further comprises a selection marker gene, and wherein each repair fragment comprises sequence for one or more genetic edits comprising a common sequence in or adjacent to one or more target loci in the microbial host cells, and wherein sequence for each of the one or more genetic edits lies between homology arms, wherein the homology arms comprise sequence homologous to sequence that flanks a target loci from the one or more target loci in the microbial host cells; (b) introducing into individual microbial host cells from step (a) a plasmid or plasmids from the pool of editing plasmids; and (c) growing the microbial host cells from step (b) in a medium selective for microbial host cells expressing the selection marker gene and isolating microbial host cells from cultures derived therefrom, thereby generating the microbial strain comprising the plurality of genetic edits.

In some cases, the plurality of genetic edits were introduced into the microbial strain by a pooled editing method, wherein the pooled method comprises: (a) combining a base population of microbial host cells with a first pool of editing plasmids, wherein each editing plasmid in the pool comprises at least one repair fragment, wherein the pool of editing plasmids comprises at least two different repair fragments, wherein each editing plasmid in the pool of editing plasmids further comprises a selection marker gene, and wherein the microbial host cells comprise one or more site-specific restriction enzymes or one or more sequences encoding one or more site-specific restriction enzymes is/are introduced into the microbial host cells along with the first pool of editing plasmids, wherein the one or more site-specific restriction enzymes target one or more target loci in the microbial host cells, wherein each repair fragment comprises sequence for one or more genetic edits comprising a common sequence in or adjacent to one or more target loci targeted by the one or more site-specific restriction enzymes, and wherein sequence for each of the one or more genetic edits lies between homology arms, wherein the homology arms comprise sequence homologous to sequence that flanks a target loci from the one or more target loci in the microbial host cell; (b) introducing into individual microbial host cells from step (a) a plasmid or plasmids from the pool of editing plasmids; and (c) growing the microbial host cells from step (b) in a medium selective for microbial host cells expressing the selection marker gene and isolating microbial host cells from cultures derived therefrom, thereby generating the microbial strain comprising the plurality of genetic edits.

In some cases, the genetic edits were introduced into the microbial strain by a pooled editing method, wherein the pooled method comprises: (a) combining a base population of microbial host cells with a first pool of editing constructs comprising one or more editing plasmids, wherein each editing plasmid in the first pool of editing constructs comprises a selection marker gene and one or both of a guide RNA (gRNA) and a repair fragment, wherein the microbial host cells comprise an RNA-guided DNA endonuclease or an RNA-guided DNA endonuclease is introduced into the microbial host cells along with the first pool of editing constructs, and wherein the first pool of editing constructs comprise: (i) gRNAs that target the same target locus or loci, and at least two different repair fragments, wherein each repair fragment comprises a sequence for one or more genetic edits comprising a common sequence in or adjacent to the target locus, and wherein sequence for each of the genetic edits lies between homology arms, wherein the homology arms comprise sequence homologous to sequence that flanks the target locus in the microbial host cell; (ii) gRNAs that target at least two different target loci, and at least two different repair fragments, wherein each repair fragment comprises a sequence for the same one or more genetic edits comprising a common sequence in or adjacent to the target loci, and wherein sequence for each of the genetic edits lies between homology arms, wherein the homology arms comprise sequence homologous to sequence that flanks the target loci in the microbial host cell; or (iii) gRNAs that target at least two different target loci, and at least two different repair fragments, wherein each repair fragment comprises a sequence for one or more genetic edits comprising a common sequence in or adjacent to the target loci, and wherein sequence for each of the genetic edits lies between homology arms, wherein the homology arms comprise sequence homologous to sequence that flanks the target loci in the microbial host cell; (b) introducing into individual microbial host cells from step (a) the first pool of editing constructs comprising the one or more editing plasmids, wherein the first pool of editing constructs comprise gRNAs and repair fragments according to any one of step (a)(i)-(iii); and (c) growing the microbial host cells from step (b) in a medium selective for microbial host cells expressing the selection marker gene and isolating microbial host cells from cultures derived therefrom, thereby generating the microbial strain comprising the plurality of genetic edits.

In some cases, the genetic edits were introduced into the microbial strain by a pooled editing method, wherein the pooled method comprises: (a) combining a base population of microbial host cells with a first pool of editing constructs comprising one or more editing plasmids, wherein each editing plasmid in the first pool of editing constructs comprises a selection marker gene and one or both of a guide RNA (gRNA) and a repair fragment, and wherein the first pool of editing constructs comprise: (i) gRNAs that target the same target locus or loci, and at least two different repair fragments, wherein each repair fragment comprises a sequence for one or more genetic edits comprising a common sequence in or adjacent to the target locus, and wherein sequence for each of the genetic edits lies between homology arms, wherein the homology arms comprise sequence homologous to sequence that flanks the target locus in the microbial host cell; (ii) gRNAs that target at least two different target loci, and at least two different repair fragments, wherein each repair fragment comprises a sequence for the same one or more genetic edits comprising a common sequence in or adjacent to the target loci, and wherein sequence for each of the genetic edits lies between homology arms, wherein the homology arms comprise sequence homologous to sequence that flanks the target loci in the microbial host cell; or (iii) gRNAs that target at least two different target loci, and at least two different repair fragments, wherein each repair fragment comprises a sequence for one or more genetic edits comprising a common sequence in or adjacent to the target loci, and wherein sequence for each of the genetic edits lies between homology arms, wherein the homology arms comprise sequence homologous to sequence that flanks the target loci in the microbial host cell; (b) introducing into individual microbial host cells from step (a) an RNA-guided DNA endonuclease and the first pool of editing constructs comprising the one or more editing plasmids, wherein the first pool of editing constructs comprise gRNAs and repair fragments according to any one of step (a)(i)-(iii); and (c) growing the microbial host cells from step (b) in a medium selective for microbial host cells expressing the selection marker gene and isolating microbial host cells from cultures derived therefrom, thereby generating the microbial strain comprising the plurality of genetic edits.

### BRIEF DESCRIPTION OF THE FIGURES

FIG. 1 depicts an embodiment of the common sequence sequencing (CS-Seq) method provided herein that entails the use of tagmentation (Nextera®) on genomic DNA extracted from microbial cells that are either wild-type or subjected to genomic editing.

FIG. 2 illustrates use of CS-Seq for enrichment of an inserted sequence (e.g. Promoter, black) and the target insertion locus (e.g. Homology Arm, gray). The CS-Seq approach can be used to identify the particular locus of insertion of one or more sequences of interest (e.g. Promoter, black) when the strains are generated in a pooled fashion.

FIG. 3 depicts an overview of an embodiment of the SG-Seq method provided herein.

FIG. 4 depicts a strategy for universal primer design where each different exogenous DNA fragment to be introduced into host cells comprise a region that is common or shared between each of the exogenous DNA fragments against which primers can be designed for use in an enrichment method provided herein.

FIG. 5 illustrates the first and second PCR steps utilized in an embodiment of the SG-Seq method provided herein for enriching genome sequence around the engineered edit.

FIG. 6 illustrates example of the frequency of annealing of semi-guided primers (highlighted) described in Example 2.

FIG. 7 depicts results of molecular analysis of amplicons obtained by the SG-Seq method provided herein using a TapeStation System (Agilent®). FIG. 7 shows that the semi-guided method allowed appropriately sized amplicons to be created that were enriched for the junction between the promoter and the locus or homology arm. Ideal range of size fragments for this application with Illumina MiSeq-based sequencing were the fragments between 200-400 bp (shown above between dashed lines).

FIG. 8 depicts an overview for detecting ectopic integrations via the enrichment sequencing methods provided herein.

FIG. 9 illustrates results of the proof of concept for ectopic integration experiment conducted in Example 3. A long-fragment library was sequenced and k-mers at varying distances downstream of the payload were detected in the raw reads. A total of 576 samples were analyzed, encompassing 32 possible edited genotypes. All samples had an independently verified on-target integration. Each data point in the plot represents the detection of a k-mer in the reads for a sample (with the corresponding count on the y-axis). As the distance downstream of the payload increases, k-mers are detected in fewer samples and with decreasing hit count. The highlighted set of points showed that on-target k-mers 100 bases downstream of the payload are detected in 58% of the samples. This would be sufficient to indicate on-target editing for homology arms as long as 99 bases. Sequencing via long-read approaches may likely increase the proportion of samples that could be successfully analyzed in this manner.

### DETAILED DESCRIPTION

### Definitions

While the following terms are believed to be well understood by one of ordinary skill in the art, the following definitions are set forth to facilitate explanation of the presently disclosed subject matter.

As used herein, the term “a” or “an” can refer to one or more of that entity, i.e. can refer to a plural referents. As such, the terms “a” or “an”, “one or more” and “at least one” can be used interchangeably herein. In addition, reference to “an element” by the indefinite article “a” or “an” does not exclude the possibility that more than one of the elements is present, unless the context clearly requires that there is one and only one of the elements.

Unless the context requires otherwise, throughout the present specification and claims, the word “comprise” and variations thereof, such as, “comprises” and “comprising” are to be construed in an open, inclusive sense that is as “including, but not limited to”.

Reference throughout this specification to “one embodiment” or “an embodiment” means that a particular feature, structure or characteristic described in connection with the embodiment may be included in at least one embodiment of the present disclosure. Thus, the appearances of the phrases “in one embodiment” or “in an embodiment” in various places throughout this specification may not necessarily all referring to the same embodiment. It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination.

As used herein, the terms “cellular organism” “microorganism” or “microbe” should be taken broadly. These terms are used interchangeably and include, but are not limited to, the two prokaryotic domains, Bacteria and Archaea, as well as certain eukaryotic fungi and protists. In some embodiments, the disclosure refers to the “microorganisms” or “cellular organisms” or “microbes” of lists/tables and figures present in the disclosure. This characterization can refer to not only the identified taxonomic genera provided herein, but also the identified taxonomic species, as well as the various novel and newly identified or designed strains of any organism provided herein.

As used herein, the term “prokaryotes” is art recognized and refers to cells that contain no nucleus or other cell organelles. The prokaryotes are generally classified in one of two domains, the Bacteria and the Archaea. The definitive difference between organisms of the Archaea and Bacteria domains is based on fundamental differences in the nucleotide base sequence in the 16S ribosomal RNA.

As used herein, the term “Archaea” refers to a categorization of organisms of the division Mendosicutes, typically found in unusual environments and distinguished from the rest of the prokaryotes by several criteria, including the number of ribosomal proteins and the lack of muramic acid in cell walls. On the basis of ssrRNA analysis, the Archaea consist of two phylogenetically-distinct groups: Crenarchaeota and Euryarchaeota. On the basis of their physiology, the Archaea can be organized into three types: methanogens (prokaryotes that produce methane); extreme halophiles (prokaryotes that live at very high concentrations of salt (NaCl); and extreme (hyper) thermophilus (prokaryotes that live at very high temperatures). Besides the unifying archaeal features that distinguish them from Bacteria (i.e., no murein in cell wall, ester-linked membrane lipids, etc.), these prokaryotes exhibit unique structural or biochemical attributes which adapt them to their particular habitats. The Crenarchaeota consists mainly of hyperthermophilic sulfur-dependent prokaryotes and the Euryarchaeota contains the methanogens and extreme halophiles.

As used herein, “bacteria” or “eubacteria” can refer to a domain of prokaryotic organisms. Bacteria include at least 11 distinct groups as follows: (1) Gram-positive (gram+) bacteria, of which there are two major subdivisions: (1) high G+C group (Actinomycetes, Mycobacteria, Micrococcus, others) (2) low G+C group (Bacillus, Clostridia, Lactobacillus, Staphylococci, Streptococci, Mycoplasmas); (2) Proteobacteria, e.g., Purple photosynthetic+non-photosynthetic Gram-negative bacteria (includes most “common” Gram-negative bacteria); (3) Cyanobacteria, e.g., oxygenic phototrophs; (4) Spirochetes and related species; (5) Planctomyces; (6) Bacteroides, Flavobacteria; (7) Chlamydia; (8) Green sulfur bacteria; (9) Green non-sulfur bacteria (also anaerobic phototrophs); (10) Radioresistant micrococci and relatives; (11) Thermotoga and Thermosipho thermophiles.

As used herein, a “eukaryote” is any organism whose cells contain a nucleus and other organelles enclosed within membranes. Eukaryotes belong to the taxon Eukarya or Eukaryota. The defining feature that sets eukaryotic cells apart from prokaryotic cells (the aforementioned Bacteria and Archaea) is that they have membrane-bound organelles, especially the nucleus, which contains the genetic material, and is enclosed by the nuclear envelope.

As used herein, the terms “genetically modified host cell,” “recombinant host cell,” and “recombinant strain” are used interchangeably herein and can refer to host cells that have been genetically modified by the iterative genetic editing methods provided herein. Thus, the terms include a host cell (e.g., bacteria, etc.) that has been genetically altered, modified, or engineered, such that it exhibits an altered, modified, or different genotype and/or phenotype (e.g., when the genetic modification affects coding nucleic acid sequences of the microorganism), as compared to the naturally-occurring organism from which it was derived. It is understood that in some embodiments, the terms refer not only to the particular recombinant host cell in question, but also to the progeny or potential progeny of such a host cell.

As used herein, the term “wild-type microorganism” or “wild-type host cell” can describe a cell that occurs in nature, i.e. a cell that has not been genetically modified.

As used herein, the term “genome” may refer to the complete set of genes or genetic material present in a cell or organism. The genome can include both the genes (the coding regions) and the noncoding DNA. The genes or genetic material may be present on a chromosome or be present on an extrachromosomal genetic element such as, for example, a plasmid, episome, mitochondria or chloroplast.

As used herein, the term “genetically engineered” may refer to any manipulation of a host cell's genome (e.g. by insertion, deletion, mutation, or replacement of nucleic acids).

As used herein, the term “control” or “control host cell” can refer to an appropriate comparator host cell for determining the effect of a genetic modification or experimental treatment. In some embodiments, the control host cell is a wild type cell. In other embodiments, a control host cell is genetically identical to the genetically modified host cell, save for the genetic modification(s) differentiating the treatment host cell. In some embodiments, the present disclosure teaches the use of parent strains as control host cells. In other embodiments, a host cell may be a genetically identical cell that lacks a specific promoter or SNP being tested in the treatment host cell.

As used herein, the term “allele(s)” can mean any of one or more alternative forms of a gene, all of which alleles relate to at least one trait or characteristic. In a diploid cell, the two alleles of a given gene occupy corresponding loci on a pair of homologous chromosomes.

As used herein, the term “locus” (loci plural) can mean any site at which an edit to the native genomic sequence is desired. In one embodiment, said term can mean a specific place or places or a site on a chromosome where for example a gene or genetic marker is found.

As used herein, the term “genetically linked” can refer to two or more traits that are co-inherited at a high rate during breeding such that they are difficult to separate through crossing.

A “recombination” or “recombination event” as used herein can refer to a chromosomal crossing over or independent assortment.

As used herein, the term “phenotype” can refer to the observable characteristics of an individual cell, cell culture, organism, or group of organisms, which results from the interaction between that individual's genetic makeup (i.e., genotype) and the environment.

As used herein, the term “chimeric” or “recombinant” when describing a nucleic acid sequence or a protein sequence can refer to a nucleic acid, or a protein sequence, that links at least two heterologous polynucleotides, or two heterologous polypeptides, into a single macromolecule, or that rearranges one or more elements of at least one natural nucleic acid or protein sequence. For example, the term “recombinant” can refer to an artificial combination of two otherwise separated segments of sequence, e.g., by chemical synthesis or by the manipulation of isolated segments of nucleic acids by genetic engineering techniques.

As used herein, a “synthetic nucleotide sequence” or “synthetic polynucleotide sequence” is a nucleotide sequence that is not known to occur in nature or that is not naturally occurring. Generally, such a synthetic nucleotide sequence can comprise at least one nucleotide difference when compared to any other naturally occurring nucleotide sequence.

As used herein, the term “nucleic acid” can refer to a polymeric form of nucleotides of any length, either ribonucleotides or deoxyribonucleotides, or analogs thereof. This term can refer to the primary structure of the molecule, and thus includes double- and single-stranded DNA, as well as double- and single-stranded RNA. It also includes modified nucleic acids such as methylated and/or capped nucleic acids, nucleic acids containing modified bases, backbone modifications, and the like. The terms “nucleic acid” and “nucleotide sequence” are used interchangeably.

As used herein, the term “gene” can refer to any segment of DNA associated with a biological function. Thus, genes can include, but are not limited to, coding sequences and/or the regulatory sequences required for their expression. Genes can also include non-expressed DNA segments that, for example, form recognition sequences for other proteins. Genes can be obtained from a variety of sources, including cloning from a source of interest or synthesizing from known or predicted sequence information, and may include sequences designed to have desired parameters.

As used herein, the term “homologous” or “homologue” or “ortholog” or “orthologue” is known in the art and can refer to related sequences that share a common ancestor or family member and are determined based on the degree of sequence identity.

The terms “homology,” “homologous,” “substantially similar” and “corresponding substantially” can be used interchangeably herein. Said terms can refer to nucleic acid fragments wherein changes in one or more nucleotide bases do not affect the ability of the nucleic acid fragment to mediate gene expression or produce a certain phenotype. These terms can also refer to modifications of the nucleic acid fragments of the instant disclosure such as deletion or insertion of one or more nucleotides that do not substantially alter the functional properties of the resulting nucleic acid fragment relative to the initial, unmodified fragment. It is therefore understood, as those skilled in the art will appreciate, that the disclosure encompasses more than the specific exemplary sequences. These terms describe the relationship between a gene found in one species, subspecies, variety, cultivar or strain and the corresponding or equivalent gene in another species, subspecies, variety, cultivar or strain. For purposes of this disclosure homologous sequences are compared.

“Homologous sequences” or “homologues” or “orthologs” are thought, believed, or known to be functionally related. A functional relationship may be indicated in any one of a number of ways, including, but not limited to: (a) degree of sequence identity and/or (b) the same or similar biological function. Preferably, both (a) and (b) are indicated. Sequence homology between amino acid or nucleic acid sequences can be defined in terms of shared ancestry. Two segments of nucleic acid can have shared ancestry because of either a speciation event (orthologs) or a duplication event (paralogs). Homology among amino acid or nucleic acid sequences can be inferred from their sequence similarity such that amino acid or nucleic acid sequences are said to be homologous if said amino acid or nucleic acid sequences share significant similarity. Significant similarity can be strong evidence that two sequences are related by divergent evolution from a common ancestor. Alignments of multiple sequences can be used to discover the homologous regions. Homology can be determined using software programs readily available in the art, such as those discussed in Current Protocols in Molecular Biology (F. M. Ausubel et al., eds., 1987) Supplement 30, section 7.718, Table 7.71. Some alignment programs are BLAST (NCBI), MacVector (Oxford Molecular Ltd, Oxford, U.K.), ALIGN Plus (Scientific and Educational Software, Pennsylvania) and AlignX (Vector NTI, Invitrogen, Carlsbad, Calif.). Another alignment program is Sequencher (Gene Codes, Ann Arbor, Mich.), using default parameters.

As used herein, the term “endogenous” or “endogenous gene,” can refer to the naturally occurring gene, in the location in which it is naturally found within the host cell genome. In the context of the present disclosure, operably linking a heterologous promoter to an endogenous gene means genetically inserting a heterologous promoter sequence in front of an existing gene, in the location where that gene is naturally present. An endogenous gene as described herein can include alleles of naturally occurring genes that have been mutated according to any of the methods of the present disclosure.

As used herein, the term “exogenous” can be used interchangeably with the term “heterologous,” and refers to a substance coming from some source other than its native source. For example, the terms “exogenous protein,” or “exogenous gene” refer to a protein or gene from a non-native source or location, and that have been artificially supplied to a biological system.

As used herein, the term “nucleotide change” refers to, e.g., nucleotide substitution, deletion, and/or insertion, as is well understood in the art. For example, mutations can contain alterations that produce silent substitutions, additions, or deletions, but do not alter the properties or activities of the encoded protein or how the proteins are made. Alternatively, mutations can be nonsynonymous substitutions or changes that can alter the amino acid sequence of the encoded protein and can result in an alteration in properties or activities of the protein.

As used herein, the term “protein modification” can refer to, e.g., amino acid substitution, amino acid modification, deletion, and/or insertion, as is well understood in the art.

As used herein, the term “at least a portion” or “fragment” of a nucleic acid or polypeptide can mean a portion having the minimal size characteristics of such sequences, or any larger fragment of the full-length molecule, up to and including the full length molecule. A fragment of a polynucleotide of the disclosure may encode a biologically active portion of a genetic regulatory element. A biologically active portion of a genetic regulatory element can be prepared by isolating a portion of one of the polynucleotides of the disclosure that comprises the genetic regulatory element and assessing activity as described herein. Similarly, a portion of a polypeptide may be 1 amino acid, 2 amino acids, 3 amino acids, 4 amino acids, 5 amino acids, 6 amino acids, 7 amino acids, and so on, going up to the full length polypeptide. The length of the portion to be used will depend on the particular application. A portion of a nucleic acid useful as a hybridization probe may be as short as 12 nucleotides; in some embodiments, it is 20 nucleotides. A portion of a polypeptide useful as an epitope may be as short as 4 amino acids. A portion of a polypeptide that performs the function of the full-length polypeptide would generally be longer than 4 amino acids.

Variant polynucleotides can also encompass sequences derived from a mutagenic and recombinogenic procedure such as DNA shuffling. Strategies for such DNA shuffling are known in the art. See, for example, Stemmer (1994) PNAS 91:10747-10751; Stemmer (1994) Nature 370:389-391; Crameri et al. (1997) Nature Biotech. 15:436-438; Moore et al. (1997) J. Mol. Biol. 272:336-347; Zhang et al. (1997) PNAS 94:4504-4509; Crameri et al. (1998) Nature 391:288-291; and U.S. Pat. Nos. 5,605,793 and 5,837,458.

For PCR amplifications disclosed herein, oligonucleotide primers can be designed for use in PCR reactions to amplify corresponding DNA sequences from cDNA or genomic DNA extracted from any organism of interest. Methods for designing PCR primers and PCR cloning are generally known in the art and are disclosed in Sambrook et al. (2001) Molecular Cloning: A Laboratory Manual (3^{rd} ed., Cold Spring Harbor Laboratory Press, Plainview, N.Y.). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Known methods of PCR include, but are not limited to, methods using paired primers, nested primers, single specific primers, degenerate primers, gene-specific primers, vector-specific primers, partially-mismatched primers, multiplex methods using multiple sets of paired primers to simultaneously amplify more than one DNA segment, and the like.

The term “primer” as used herein can refer to an oligonucleotide which is capable of annealing to the amplification target allowing a DNA polymerase to attach, thereby serving as a point of initiation of DNA synthesis when placed under conditions in which synthesis of primer extension product is induced, i.e., in the presence of nucleotides and an agent for polymerization such as DNA polymerase and at a suitable temperature and pH. The (amplification) primer can be single stranded for maximum efficiency in amplification. The primer can be an oligodeoxyribonucleotide. The primer must be sufficiently long to prime the synthesis of extension products in the presence of the agent for polymerization. The exact lengths of the primers will depend on many factors, including temperature and composition (A/T vs. G/C content) of primer. A pair of bi-directional primers consists of one forward and one reverse primer as commonly used in the art of DNA amplification such as in PCR amplification.

As used herein, “promoter” can refer to a DNA sequence capable of controlling the expression of a coding sequence or functional RNA. In some embodiments, the promoter sequence consists of proximal and more distal upstream elements, the latter elements often referred to as enhancers. Accordingly, an “enhancer” can be a DNA sequence that can stimulate promoter activity, and may be an innate element of the promoter or a heterologous element inserted to enhance the level or tissue specificity of a promoter. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. For example, promoters can be used to change the level of expression of a gene in a manner that is constitutive or that responds to an endogenous or exogenous stimulus. It is further recognized that since in most cases the exact boundaries of regulatory sequences have not been completely defined, DNA fragments of some variation may have identical promoter activity.

As used herein, the phrases “recombinant construct”, “expression construct”, “chimeric construct”, “construct”, and “recombinant DNA construct” can be used interchangeably herein. A recombinant construct can comprise an artificial combination of nucleic acid fragments, e.g., regulatory and coding sequences that are not found together in nature. For example, a chimeric construct may comprise regulatory sequences and coding sequences that are derived from different sources, or regulatory sequences and coding sequences derived from the same source, but arranged in a manner different than that found in nature. Such construct may be used by itself or may be used in conjunction with a vector. If a vector is used then the choice of vector is dependent upon the method that will be used to transform host cells as is well known to those skilled in the art. For example, a plasmid vector can be used. The skilled artisan is well aware of the genetic elements that must be present on the vector in order to successfully transform, select and propagate host cells comprising any of the isolated nucleic acid fragments of the disclosure. The skilled artisan will also recognize that different independent transformation events will result in different levels and patterns of expression (Jones et al., (1985) EMBO J. 4:2411-2418; De Almeida et al., (1989) Mol. Gen. Genetics 218:78-86), and thus that multiple events must be screened in order to obtain lines displaying the desired expression level and pattern. Such screening may be accomplished by direct sequencing, Southern analysis of DNA, Northern analysis of mRNA expression, immunoblotting analysis of protein expression, or phenotypic analysis, among others. Vectors can be plasmids, viruses, bacteriophages, pro-viruses, phagemids, transposons, artificial chromosomes, and the like, that replicate autonomously or can integrate into a chromosome of a host cell. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that is not autonomously replicating. As used herein, the term “expression” refers to the production of a functional end-product e.g., an mRNA or a protein (precursor or mature).

“Operably linked” or “functionally linked” can mean the sequential arrangement of any functional genetic element according to the disclosure (e.g., promoter, terminator, degron, solubility tag, etc.) with a further oligo- or polynucleotide. In some cases, the sequential arrangement can result in transcription of said further polynucleotide. In some cases, the sequential arrangement can result in translation of said further polynucleotide. The functional genetic elements can be present upstream or downstream of the further oligo or polynucleotide. In one example, “operably linked” or “functionally linked” can mean a promoter controls the transcription of the gene adjacent or downstream or 3′ to said promoter. In another example, “operably linked” or “functionally linked” can mean a terminator controls termination of transcription of the gene adjacent or upstream or 5′ to said terminator.

The term “product of interest” or “biomolecule” as used herein can refer to any product produced by microbes from feedstock. In some cases, the product of interest may be a small molecule, enzyme, peptide, amino acid, organic acid, synthetic compound, fuel, alcohol, etc. For example, the product of interest or biomolecule may be any primary or secondary extracellular metabolite. The primary metabolite may be, inter alia, ethanol, citric acid, lactic acid, glutamic acid, glutamate, lysine, threonine, tryptophan and other amino acids, vitamins, polysaccharides, etc. The secondary metabolite may be, inter alia, an antibiotic compound like penicillin, or an immunosuppressant like cyclosporin A, a plant hormone like gibberellin, a statin drug like lovastatin, a fungicide like griseofulvin, etc. The product of interest or biomolecule may also be any intracellular component produced by a microbe, such as: a microbial enzyme, including: catalase, amylase, protease, pectinase, glucose isomerase, cellulase, hemicellulase, lipase, lactase, streptokinase, and many others. The intracellular component may also include recombinant proteins, such as insulin, hepatitis B vaccine, interferon, granulocyte colony-stimulating factor, streptokinase and others.

As used herein, the term “HTP genetic design library” or “library” refers to collections of genetic perturbations according to the present disclosure. In some embodiments, the libraries of the present invention may manifest as i) a collection of sequence information in a database or other computer file, ii) a collection of genetic constructs comprising the aforementioned series of genetic elements, or iii) host cell strains comprising said genetic elements. In some embodiments, the libraries of the present disclosure may refer to collections of individual elements (e.g., collections of promoters for PRO swap libraries, collections of terminators for STOP swap libraries, collections of protein solubility tags for SOLUBILITY TAG swap libraries, or collections of protein degradation tags for DEGRADATION TAG swap libraries). In other embodiments, the libraries of the present disclosure may also refer to combinations of genetic elements, such as combinations of promoter:genes, gene:terminator, or even promoter:gene:terminators. In some embodiments, the libraries of the present disclosure may also refer to combinations of promoters, terminators, protein solubility tags and/or protein degradation tags. In some embodiments, the libraries of the present disclosure further comprise metadata associated with the effects of applying each member of the library in host organisms. For example, a library as used herein can include a collection of promoter::gene sequence combinations, together with the resulting effect of those combinations on one or more phenotypes in a particular species, thus improving the future predictive value of using said combination in future promoter swaps.

As used herein, the term “SNP” can refer to Small Nuclear Polymorphism(s). In some embodiments, SNPs of the present disclosure should be construed broadly, and include single nucleotide polymorphisms, sequence insertions, deletions, inversions, and other sequence replacements. As used herein, the term “non-synonymous” or “non-synonymous SNPs” can refer to mutations that lead to coding changes in host cell proteins.

A “high-throughput (HTP)” method of genomic engineering may involve the utilization of at least one piece of equipment that enables one to evaluate a large number of experiments or conditions, for example, automated equipment (e.g. a liquid handler or plate handler machine) to carry out at least one-step of said method.

The term “polynucleotide” as used herein can encompass oligonucleotides and refers to a nucleic acid of any length. Polynucleotides may be DNA or RNA. Polynucleotides may be single-stranded (ss) or double-stranded (ds) unless otherwise specified. Polynucleotides may be synthetic, for example, synthesized in a DNA synthesizer, or naturally occurring, for example, extracted from a natural source, or derived from cloned or amplified material. Polynucleotides referred to herein can contain modified bases or nucleotides.

The term “pool”, as used herein, can refer to a collection of at least 2 polynucleotides. A pool of polynucleotides may comprise a plurality of different polynucleotides. In some embodiments, a set of polynucleotides in a pool may comprise at least 5, at least 10, at least 12 or at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 200, at least 300, at least 400, at least 500, at least 600, at least 700, at least 800, at least 900, or at least 1000 or more polynucleotides.

As used herein, the term “assembling”, can refer to a reaction in which two or more, four or more, six or more, eight or more, ten or more, 12 or more 15 or more polynucleotides, e.g., four or more polynucleotides are joined to another to make a longer polynucleotide.

As used herein, the term “incubating under suitable reaction conditions”, can refer to maintaining a reaction a suitable temperature and time to achieve the desired results, i.e., polynucleotide assembly. Reaction conditions suitable for the enzymes and reagents used in the present method are known (e.g. as described in the Examples herein) and, as such, suitable reaction conditions for the present method can be readily determined. These reactions conditions may change depending on the enzymes used (e.g., depending on their optimum temperatures, etc.).

As used herein, the term “joining”, can refer to the production of covalent linkage between two sequences.

As used herein, the term “composition” can refer to a combination of reagents that may contain other reagents, e.g., glycerol, salt, dNTPs, etc., in addition to those listed. A composition may be in any form, e.g., aqueous or lyophilized, and may be at any state (e.g., frozen or in liquid form).

As used herein a “vector” is a suitable DNA into which a fragment or DNA assembly may be integrated such that the engineered vector can be replicated in a host cell. A linearized vector may be created restriction endonuclease digestion of a circular vector or by PCR. The concentration of fragments and/or linearized vectors can be determined by gel electrophoresis or other means.

As used herein, the term “integron” can refer to a mobile genetic element or a genetic element integrated into a nucleic acid (e.g., a genome, plasmid, etc.) that comprises or contains a gene cassette comprising an exogenous gene, a gene encoding an integron integrase (Intl), an integron-associated recombination site (attl) and an integron-associated promoter (Pc) as described in Gillings, Michael R, “Integrons: Past, Present, and Future” Microbiology and Molecular Biology Review, June 2014 Vol. 78:2, pp. 257-277, the contents of which are herein incorporated by reference.

### Overview

Provided herein are methods, compositions and kits for genotyping organisms engineered to possess one or more genetic edits using targeted enrichment coupled with sequencing (e.g., next generation sequencing (NGS)). The methods, compositions and kits provided herein can be particularly useful in instances when screening by polymerase chain reaction (PCR) with primers targeting specific genetic edits is impractical due to a large number of possible loci where the edit could be located within an organism's genome, and when multiple PCR reactions per sample would need to be performed to assess the genotype of the organism. In one embodiment, the enrichment methods provided herein are designed to work with sequences that are inserted into the genome of an organism to provide a common priming site for PCR-based genome enrichment for subsequent sequencing (e.g., next generation sequencing (NGS)). In one embodiment, while the enrichment methods provided herein use sequencing technology (e.g., NGS), they do not require the entire genome of an organism to be sequenced. In one aspect, provided herein is a targeted enrichment method referred to as common-sequence-sequencing (CS-seq) that generally entails amplifying genomic regions of interest from an organism (e.g., microbe) using a first primer that binds sequence present in a genetic edit introduced into the genome of the organism and a second primer that binds the nearest universal sequence present on an adapter introduced during preparation of the genomic DNA for use in the method and subsequently analyzing amplicons generated during the method via sequencing. In another aspect, provided herein is a targeted enrichment method referred to as semi-guided-sequencing (SG-seq) that generally entails amplifying genomic regions of interest from an organism (e.g., microbe) using a first primer that binds sequence present in a genetic edit introduced into the genome of the organism and a second primer that binds the nearest universal sequence present on a 5′tail of a semi-guided or partially degenerate primer that anneals at different distances on a genetic element (e.g., chromosome) using a variable locus-specific sequence at its 3′ end introduced during preparation of the genomic DNA for use in the method and subsequently analyzing amplicons generated during the method via sequencing.

The enrichment methods provided herein (e.g., CS-seq or SG-seq) can be implemented in a high-throughput manner. The enrichment methods provided herein (e.g., CS-seq or SG-seq) can be implemented as part of the workflow in any high-throughput method for engineering organisms known in the art such as, for example, the high-throughput engineering methods described in U.S. Pat. No. 9,988,624, WO2018226880, WO2018226900 and WO2018126207, each of which are herein incorporated by reference in their entirety. In one embodiment, the enrichment methods provided herein (e.g., CS-seq or SG-seq) enrich only for the regions of the genome of an organism that are required to make the genotype determination. Accordingly, the enrichment methods provided herein (e.g., CS-seq or SG-seq) can vastly decrease sequencing costs as compared to whole genome sequencing methods when screening organisms for genetic edits.

In one embodiment, the enrichment methods provided herein (e.g., CS-seq and/or SG-seq) are used for screening and genotyping the genomes of organisms that have been edited. The organisms suitable for use in the enrichment methods provided herein (e.g., CS-seq or SG-seq) may be any prokaryotic or eukaryotic organism know in the art and/or provided herein. As provided herein, the genome of the organism can encompass both the chromosomal and extrachromosomal genetic elements present in the cells of the organism. The genetic edits in the genome of an organism may have been introduced by any method known in the art for introducing genetic edits. The methods utilized for introducing genetic edits in the genome of an organism can be selected from the group consisting of homologous recombination, nuclease-based editing (e.g. CRISPR/Cas9, transcription activator-like effector nucleases (TALEN), Meganuclease, Zn-finger) with a targeted donor sequence, lambda red recombination, viral or phage transduction or any combination thereof.

The enrichment methods provided herein (e.g., CS-seq or SG-seq) can be used to genotype an organism that has been subjected to genetic engineering. The genetic engineering can entail the introduction of one or a plurality of genetic edits into the genome of the organism. The one or a plurality of genetic edits can be novel or exogenous sequences. The one or plurality of genetic edits can be introduced or inserted using homologous recombination-based editing or CRISP-Cas9 based editing. The enrichment methods provided herein (e.g., CS-seq or SG-seq) can be useful for genotyping a mixed population of edited organisms where any organism in the population could be wild type or edited at one locus or multiple loci. In one embodiment, the enrichment methods provided herein (e.g., CS-seq or SG-seq) are used to genotype or identify or confirm the loci of multiple genetic edits in the genome of a microbial strain. The multiple genetic edits may have been introduced simultaneously (e.g., where a subset of possible edits occur in individually isolated colonies), iteratively (e.g., where at each step either a single specified edit or a pool of possible edits (i.e. from a library) are possible), synthetically (e.g., genome shuffling), via natural recombination (e.g., mating) or any combination thereof.

In one embodiment, the enrichment methods provided herein (e.g., CS-seq or SG-seq) are used to identify off-target insertion sites of genetic edits in the genome of an organism. Off-target insertion or “ectopic” insertion/recombination of genetic edits can be frequent in some organisms, such as, for example, organisms with low rates of homologous recombination. When introducing libraries of genetic edits into organisms that comprise low rates of homologous recombination (e.g. via homology-directed recombination, CRISPR-Cas9, etc.), the enrichment methods provided herein (e.g., CS-seq or SG-seq) can be used to identify the resulting clones that received a genetic edit in the intended target locus or site rather than an off-target insertion of said genetic edit. The enrichment methods provided herein (e.g., CS-seq or SG-seq) can be used to distinguish between the following outcomes: (1) no edit occurred, (2) editing occurred at the intended site, (3) editing occurred at an unintended site, and (4) editing occurred at both intended and unintended sites. In one embodiment, the enrichment-based genotyping methods provided herein can help distinguish between (2), (3), and (4) to allow identification of strains of type (2). In one embodiment, in order to distinguish from these possibilities, the fragment size of the libraries generated during the enrichment processes provided herein are longer than the homology arms used for integration to allow for identification of the site where integration occurred. The enrichment methods provided herein (e.g., CS-seq or SG-seq) can generate fragment libraries with an average length of up to 476 base pairs (bps), which can allow for reliable detection of ectopic integration with homology arms of ˜100 bp or shorter.

In another embodiment, the enrichment methods provided herein (e.g., CS-seq or SG-seq) are used to identify the presence of desired vs. unwanted genomic rearrangements in an organism. Known natural variations or mutations in the genome of the cells of an organism that can occur due to movement of transposons or natural genomic rearrangement can be identified using the enrichment methods provided herein. In one embodiment, the enrichment methods provided herein (e.g., CS-seq or SG-seq) are used to identify natural variations or rearrangements alone or in combination with identifying genetic edits introduced into the genome of an organism using any of the genetic engineering methods known in the art and/or provided herein.

### Common Sequence-Sequencing (CS-Seq) Enrichment Method

In one embodiment, the CS-seq method provided herein for identifying one or a plurality of genetic edits introduced into a microbial strain comprises: (a) appending an adaptor comprising a universal sequence to nucleic acid fragments from a plurality of nucleic acid fragments prepared from nucleic acid derived from a microbial strain, wherein the microbial strain comprises the one or the plurality of genetic edits, wherein each genetic edit from the one or the plurality of genetic edits comprises a common sequence; (b) amplifying each of the nucleic acid fragments from step (a) in a polymerase chain reaction (PCR) using a primer pair comprising a first primer comprising a sequence complementary to the common sequence at its 3′ end and a 5′ tail comprising non-complementary sequence and a second primer comprising sequence complementary to the universal sequence at its 3′ end and a 5′ tail comprising non-complementary sequence, wherein the non-complementary sequence of the first primer and the second primer each comprise sequencing primer binding sites; and (c) performing molecular analysis on amplicons generated from the PCR performed in step (b), thereby identifying the one or the plurality of genetic edits in the microbial strain. The sequencing primer binding sites of the non-complementary sequence of the first and/or second primer can be replaced with an adapter sequence compatible with a third generation sequencing platform (e.g., Oxford Nanopore MinION sequencing platform). The sequencing primer binding sites of the non-complementary sequence of the first and/or second primer further comprise an adapter sequence compatible with a third generation sequencing platform (e.g., Oxford Nanopore MinION sequencing platform). In one embodiment, the first primer is specific to a genetic edit and the second primer is specific to a single universal sequence found in each adapter. In one embodiment, the non-complementary sequence of the first primer and/or the second primer further comprise a sample specific index sequence. In some cases, the one or the plurality of genetic edits is in an episome, chromosome, or other genomic DNA. In some cases, the chromosome is from bacteria or fungi. In some cases, size selection can be performed after each step in the method. In one embodiment, the molecular analysis comprises amplicon size selection on the amplicons generated from the PCR performed in step (b). Size selection can comprise any method known in the art for performing size selection such as, for example, column purification or isolation from an agarose gel. Size selection can comprise digestion and/or gel electrophoresis, optionally, wherein the electrophoresis is preceded by the digestion. In one embodiment, the amplicon size selection comprises digestion and/or gel electrophoresis of the amplicons, optionally, wherein the electrophoresis is preceded by the digestion. In some cases, size selection can be performed using SPRI beads or magnetic particles coated with carboxyl groups (in the form of succinic acid) that can bind DNA non-specifically and reversibly. Amplicon size selection can be employed to isolate amplicons sizes that are compatible with a sequencing platform or technology used for the molecular analysis. The amplicon size selection can isolate fragments that are at least 50 base pairs (bps), 75 bps, 100 bps, 125 bps, 150 bps, 175 bps, 200 bps, 225 bps, 250 bps, 275 bps, 300 bps, 325 bps, 350 bps, 375 bps, 400 bps, 425 bps, 450 bps, 475 bps, or 500 bps.

In another embodiment, the CS-seq method provided herein for identifying one or a plurality of genetic edits introduced into a microbial strain comprises: (a) appending an adaptor comprising a universal sequence to nucleic acid fragments from a plurality of nucleic acid fragments prepared from nucleic acid derived from a microbial strain, wherein the microbial strain comprises the one or the plurality of genetic edits, wherein each genetic edit from the one or the plurality of genetic edits comprises a common sequence; (b) amplifying each of the nucleic acid fragments from step (a) in a first polymerase chain reaction (PCR) using a first primer pair comprising a first primer comprising a sequence complementary to the common sequence at its 3′ end and a 5′ tail comprising non-complementary sequence and a second primer comprising sequence complementary to the universal sequence at its 3′ end and a 5′ tail comprising non-complementary sequence; (c) amplifying amplicons generated in step (b) in a second PCR using a second primer pair comprising a first primer comprising a 3′ end comprising sequence complementary to the non-complementary sequence in the 5′ tail of the first primer from the first primer pair and a second primer comprising a 3′ end comprising sequence complementary to the non-complementary sequence in the 5′ tail of the second primer from the first primer pair, wherein the first primer and the second primer from the second primer pair each comprise 5′ tails comprising non-complementary sequence that each comprise sequencing primer binding sites; and (d) performing molecular analysis on amplicons generated from the PCR performed in step (c), thereby identifying the one or the plurality of genetic edits in the microbial strain. The sequencing primer binding sites of the non-complementary sequence of the first and/or second primer of the second primer pair can be replaced with an adapter sequence compatible with a third generation sequencing platform (e.g., Oxford Nanopore Technologies MinION sequencing platform). The sequencing primer binding sites of the non-complementary sequence of the first and/or second primer of the second primer pair further comprise an adapter sequence compatible with a third generation sequencing platform (e.g., Oxford Nanopore Technologies MinION sequencing platform). In one embodiment, the first primer is specific to a genetic edit and the second primer of the second primer pair is specific to a single universal sequence found in each adapter. In one embodiment, the non-complementary sequence of the first primer and/or the second primer of the second primer pair further comprise a sample specific index sequence. The one or the plurality of genetic edits can be in a bacterial chromosome, plasmid or episome. In some cases, size selection can be performed after each step in the method. In one embodiment, the molecular analysis comprises amplicon size selection on the amplicons generated from the PCR performed in step (c). Size selection can comprise any method known in the art for performing size selection such as, for example, column purification or isolation from an agarose gel. Size selection can comprise digestion and/or gel electrophoresis, optionally, wherein the electrophoresis is preceded by the digestion. In one embodiment, the amplicon size selection comprises digestion and/or gel electrophoresis of the amplicons, optionally, wherein the electrophoresis is preceded by the digestion. In some cases, size selection can be performed using SPRI beads or magnetic particles coated with carboxyl groups (in the form of succinic acid) that can bind DNA non-specifically and reversibly. Amplicon size selection can be employed to isolate amplicons sizes that are compatible with a sequencing platform or technology used for the molecular analysis. The amplicon size selection can isolate fragments that are at least 50 base pairs (bps), 75 bps, 100 bps, 125 bps, 150 bps, 175 bps, 200 bps, 225 bps, 250 bps, 275 bps, 300 bps, 325 bps, 350 bps, 375 bps, 400 bps, 425 bps, 450 bps, 475 bps, or 500 bps.

Also provided herein is a composition for use in a CS-seq enrichment method provided herein. The composition can comprise a one or more adapters comprising the universal sequence and at least one primer pair. The at least one primer pair can comprise a first primer comprising a sequence complementary to a common sequence present in a genetic edit at the primer's 3′ end and a 5′ tail comprising non-complementary sequence and a second primer comprising sequence complementary to the universal sequence at its 3′ end and a 5′ tail comprising non-complementary sequence. The non-complementary sequence of the first primer and the second primer can each comprise sequencing primer binding sites. The non-complementary sequence of the first and/or second primer can each comprise an adapter sequence compatible with a third generation sequencing platform (e.g., Oxford Nanopore Technologies MinION sequencing platform). In some cases, the composition can further comprise a second primer pair comprising a first primer comprising a 3′ end comprising sequence complementary to the non-complementary sequence in the 5′ tail of the first primer from the first primer pair and a second primer comprising a 3′ end comprising sequence complementary to the non-complementary sequence in the 5′ tail of the second primer from the first primer pair. In one embodiment, the first primer and the second primer from the second primer pair each comprise 5′ tails comprising non-complementary sequence that each comprise sequencing primer binding sites. The non-complementary sequence of the first and/or second primer from the second primer pair can each comprise an adapter sequence compatible with a third generation sequencing platform (e.g., Oxford Nanopore Technologies MinION sequencing platform). In one embodiment, the composition further comprises reagents necessary for performing tagmentation. In another embodiment, the composition further comprises one or more reagents for performing nucleic extraction, purification, ligation, PCR, size selection or sequencing.

In one embodiment, derivation of the nucleic acid for use in a CS-seq method provided herein entails lysing the microbial strain. Lysing of the microbial strain can performed using any method known in the art for lysing cells such as, for example, temperature based methods (e.g., boil preparation, freeze-thawing, etc.), physical or mechanical means (e.g., grinding, sonication), pressure-based methods (e.g., French press) or enzymatic or chemical means (e.g., alcohols, ether, and chloroform, chelating agents (EDTA), detergents or surfactants (e.g., SDS, Triton) and chaotropic agents (e.g., urea, guanidine)). In some cases, derivation can further comprise isolating the nucleic acid from the microbial strain. The isolating can entail extracting nucleic acid (e.g., genomic DNA) from the microbial strain and purifying the extracted nucleic acid. Purification of the nucleic acid can be performed using any nucleic acid purification method known in the art. In one embodiment, the derivation of the nucleic acid entails performing a boil preparation of the microbial strain.

In one embodiment, the derivation of the nucleic acid entails whole genome amplification (WGA) or multiple displacement amplification (MDA) of nucleic acid isolated from the microbial strain.

In one embodiment, adapters are appended to nucleic acid derived from the microbial strain via a transposon mediated adapter addition reaction. The transposon mediated adapter addition reaction can be any such method known in the art. In some cases, adapters are appended to nucleic acid derived from the microbial strain via a tagmentation reaction. In one embodiment, the nucleic acid derived from the microbial strain is fragmented and adapters comprising the universal sequence are ligated to the nucleic acid fragments. Ligation can be facilitated through the use of enzymes (i.e. T4 DNA ligase) and methods known in the art, including, but not limited to, commercially available kits such as the Encore™ Ultra Low Input NGS Library System.

In one embodiment, fragmentation of the nucleic acids can be achieved through methods known in the art. Fragmentation can be through physical fragmentation methods and/or enzymatic fragmentation methods. Physical fragmentation methods can include nebulization, sonication, and/or hydrodynamic shearing. In some embodiments, the fragmentation can be accomplished mechanically comprising subjecting the nucleic acids in the input sample to acoustic sonication. In some embodiments, the fragmentation comprises treating the nucleic acids in the input sample with one or more enzymes under conditions suitable for the one or more enzymes to generate double-stranded nucleic acid breaks. Examples of enzymes useful in the generation of nucleic acid or polynucleotide fragments include sequence specific and non-sequence specific nucleases. Non-limiting examples of nucleases include DNase I, Fragmentase, restriction endonucleases, variants thereof, and combinations thereof. Reagents for carrying out enzymatic fragmentation reactions are commercially available (e.g., from New England Biolabs). For example, digestion with DNase I can induce random double-stranded breaks in DNA in the absence of Mg⁺⁺ and in the presence of Mn⁺⁺. In some embodiments, fragmentation comprises treating the nucleic acids in the input sample with one or more restriction endonucleases. Fragmentation can produce fragments having 5′ overhangs, 3′ overhangs, blunt ends, or a combination thereof. In some embodiments, such as when fragmentation comprises the use of one or more restriction endonucleases, cleavage of sample polynucleotides leaves overhangs having a predictable sequence.

In one embodiment, the molecular analysis of the amplicons in the CS-seq methods provided herein comprises DNA sequencing using sequencing primers directed to the sequencing primer binding sites. The molecular analysis can comprises any first, second, or third generation DNA sequencing method known in the art and/or provided herein. In one embodiment, the molecular analysis further comprises comparing sequence reads obtained from the sequencing of the amplicons to a reference database for the microbial strain using a computer-implemented method, thereby identifying the one or the plurality of genetic edits. The computer-implemented method can utilize a sequence similarity search program, a sequence composition search program or a combination thereof. The sequence similarity search program can employ a basic local alignment search tool (BLAST) algorithm, fuzzy logic, lowest common ancestor (LCA) algorithm or a profile hidden Markov Model (pHMM). The sequence composition search program employs interpolated Markov models (IMMs), naive Bayesian classifiers, k-mers or k-means/k-nearest-neighbor algorithms. In one embodiment, the sequence composition search program employs k-mers. The k-mers can comprise short nucleotide sequences comprising nucleotide bases complementary to a sequence near the one or each of the plurality of genetic edits. In one embodiment, detection of the short nucleotide sequence in the sequence reads indicates presence of the one or each of the plurality of genetic edits in the microbial strain. The sequence near the one or each of the plurality of genetic edits is within 25 base pairs (bps), 20 bps, 15 bps, 10 bps, or 5 bps of the one or each of the plurality of genetic edits.

In one embodiment, the common sequence in at least one genetic edit in the plurality of genetic edits is different from the common sequence in each other genetic edit in the plurality of genetic edits. In another embodiment, the common sequence in each genetic edit in the plurality of genetic edits is different from the common sequence in each other genetic edit in the plurality of genetic edits. In yet another embodiment, the common sequence is specific to a genetic edit. In one embodiment, the common sequence is a portion of the sequence that makes up the genetic edit. The portion of the sequence that makes up the genetic edit that can serve as the common sequence can be at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95 or 100 nucleotides in length. The portion of the sequence that makes up the genetic edit that can serve as the common sequence can be at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95 or 100 nucleotides in length. The portion of the sequence that makes up the genetic edit that can serve as the common sequence can be between 1-5, between 5-10, between 10-15, between 15-20, between 20-25, between 25-30, between 30-35, between 35-40, between 40-45, between 45-50, between 50-55, between 55-60, between 60-65, between 65-70, between 70-75, between 75-80, between 80-85, between 85-90, between 90-95 or between 95-100 nucleotides in length. The portion of the sequence that makes up the genetic edit that can serve as the common sequence can be from 1-5, from 5-10, from 10-15, from 15-20, from 20-25, from 25-30, from 30-35, from 35-40, from 40-45, from 45-50, from 50-55, from 55-60, from 60-65, from 65-70, from 70-75, from 75-80, from 80-85, from 85-90, from 90-95 or from 95-100 nucleotides in length, inclusive of the endpoints. In another embodiment, the common sequence is sequence added to the genetic edit that does not alter or affect the function of the genetic edit. In some cases, the common sequence added to the genetic edit can be shared with at least one genetic edit in the plurality of genetic edits. In some cases, the common sequence added to the genetic edit can be shared with each of the genetic edits in the plurality of genetic edits. The common sequence added to the genetic edit can be at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95 or 100 nucleotides in length. The common sequence added to the genetic edit can be at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95 or 100 nucleotides in length. The common sequence added to the genetic edit can be between 1-5, between 5-10, between 10-15, between 15-20, between 20-25, between 25-30, between 30-35, between 35-40, between 40-45, between 45-50, between 50-55, between 55-60, between 60-65, between 65-70, between 70-75, between 75-80, between 80-85, between 85-90, between 90-95 or between 95-100 nucleotides in length. The common sequence added to the genetic edit can be from 1-5, from 5-10, from 10-15, from 15-20, from 20-25, from 25-30, from 30-35, from 35-40, from 40-45, from 45-50, from 50-55, from 55-60, from 60-65, from 65-70, from 70-75, from 75-80, from 80-85, from 85-90, from 90-95 or from 95-100 nucleotides in length, inclusive of the endpoints. The common sequence and/or genetic edit (of which the common sequence can be all or a part of) can be selected from any genetic element including a promoter sequence, a termination sequence, a degron sequence, a protein solubility tag sequence, a protein degradation tag sequence, a ribosomal binding site (RBS) sequence, a landing pad primer binding sequence, an antibiotic resistance gene sequence or any portion thereof. The common sequence and/or genetic edit (of which the common sequence can be all or a part of) can be an exogenous gene sequence or mutated version thereof. The common sequence and/or genetic edit (of which the common sequence can be all or a part of) can be a mutated version of a gene present in the genome of the organism. The mutated version of the gene sequence can contain or comprise a single nucleotide polymorphism (SNP). In one embodiment, the one genetic edit or the plurality of genetic edits introduced into an organism that is subsequently subjected to a CS-seq enrichment method provided herein can be derived from or introduced as a part of a library of genetic edits. The library of genetic edits can be libraries of a genetic element, including promoter sequences, termination sequences, solubility tag sequences, degradation tag sequences or SNP sequences that can be generated using any of the methods described in WO 2020/092704, WO 2018/226900, WO 2018/226880 or WO 2017/100377, each of which is herein incorporated by reference in their entireties. Said libraries of promoter sequences, termination sequences, solubility tag sequences, degradation tag sequences or SNP sequences can be introduced using the promoter swapping, terminator (stop) swapping, solubility tag swapping, degradation tag swapping or SNP swapping methods described in WO 2018/226900, WO 2018/226880 or WO 2017/100377. In one embodiment, the common sequence and/or genetic edit (of which the common sequence can be all or a part of) is not a transposon or transposon-related sequence.

### Semi-Guided-Sequencing (SG-Seq) Enrichment Method

In one embodiment, the SG-seq method provided herein for identifying one or a plurality of genetic edits introduced into a microbial strain comprises: (a) amplifying nucleic acid derived from a microbial strain in a first polymerase chain reaction (PCR), wherein the microbial strain comprises the one or the plurality of genetic edits, and wherein each genetic edit from the one or the plurality of genetic edits comprises a common sequence, wherein the first PCR utilizes a first primer pair comprising a first primer comprising a sequence complementary to the common sequence at its 3′ end and a 5′ tail comprising a first universal sequence and a plurality of second primers comprising a priming sequence complementary to a variable locus-specific sequence at its 3′ end and a 5′ tail comprising a second universal sequence that is common among all second primers; (b) amplifying amplicons generated in step (a) in a second PCR using a second primer pair comprising a first primer comprising a 3′ end comprising sequence complementary to the first universal sequence in the 5′ tail of the first primer from the first primer pair and a second primer comprising a 3′ end comprising sequence complementary to the second universal sequence in the 5′ tail of each of the second primers from the first primer pair, wherein the first primer and the second primer from the second primer pair each comprise 5′ tails comprising non-complementary sequence that each comprise sequencing primer binding sites; and (c) performing molecular analysis on the amplicons generated from the second PCR performed in step (b), thereby identifying the one or the plurality of genetic edits in the microbial strain. The sequencing primer binding sites of the non-complementary sequence of the first and/or second primer of the second primer pair can be replaced with an adapter sequence compatible with a third generation sequencing platform (e.g., Oxford Nanopore Technologies MinION sequencing platform). The sequencing primer binding sites of the non-complementary sequence of the first and/or second primer of the second primer pair further comprise an adapter sequence compatible with a third generation sequencing platform (e.g., Oxford Nanopore Technologies MinION sequencing platform). In one embodiment, the non-complementary sequence of the first primer and/or the second primer of the second primer pair further comprise a sample specific index sequence. In some cases, the one or the plurality of genetic edits is in an episome, chromosome, or other genomic DNA. In some cases, the chromosome is from bacteria or fungi. In some cases, size selection can be performed after each step in the method. In one embodiment, the molecular analysis comprises amplicon size selection on the amplicons generated from the PCR performed in step (b). Size selection can comprise any method known in the art for performing size selection such as, for example, column purification or isolation from an agarose gel. Size selection can comprise digestion and/or gel electrophoresis, optionally, wherein the electrophoresis is preceded by the digestion. In one embodiment, the amplicon size selection comprises digestion and/or gel electrophoresis of the amplicons, optionally, wherein the electrophoresis is preceded by the digestion. In some cases, size selection can be performed using SPRI beads or magnetic particles coated with carboxyl groups (in the form of succinic acid) that can bind DNA non-specifically and reversibly. Amplicon size selection can be employed to isolate amplicons sizes that are compatible with a sequencing platform or technology used for the molecular analysis. The amplicon size selection can isolate fragments that are at least 50 base pairs (bps), 75 bps, 100 bps, 125 bps, 150 bps, 175 bps, 200 bps, 225 bps, 250 bps, 275 bps, 300 bps, 325 bps, 350 bps, 375 bps, 400 bps, 425 bps, 450 bps, 475 bps, 500 bps, 550 bps, 600 bps, 650 bps, 700 bps, 750 bps, 800 bps, 850 bps, 900 bps, 950 bps or 1000 bps.

In one embodiment, the SG-seq method provided herein for identifying one or a plurality of genetic edits introduced into a microbial strain comprises: (a) amplifying nucleic acid derived from a microbial strain in a polymerase chain reaction (PCR), wherein the microbial strain comprises the one or the plurality of genetic edits, and wherein each genetic edit from the one or the plurality of genetic edits comprises a common sequence, wherein the PCR utilizes a primer pair comprising a first primer comprising a sequence complementary to the common sequence at its 3′ end and a 5′ tail comprising a first universal sequence and a plurality of second primers comprising a priming sequence complementary to a variable locus-specific sequence at its 3′ end and a 5′ tail comprising a second universal sequence that is common among all second primers, wherein the first primer and each second primer of the plurality of second primers each comprise sequencing primer binding sites in the 5′ tail; and (b) performing molecular analysis on amplicons generated from the PCR performed in step (a), thereby identifying the one or the plurality of genetic edits in the microbial strain. The sequencing primer binding sites of the non-complementary sequence of the first and/or second primer can be replaced with an adapter sequence compatible with a third generation sequencing platform (e.g., Oxford Nanopore Technologies MinION sequencing platform). The sequencing primer binding sites of the non-complementary sequence of the first and/or second primer further comprise an adapter sequence compatible with a third generation sequencing platform (e.g., Oxford Nanopore Technologies MinION sequencing platform). In one embodiment, the non-complementary sequence of the first primer and/or the second primer further comprise a sample specific index sequence. In some cases, the one or the plurality of genetic edits is in an episome, chromosome, or other genomic DNA. In some cases, the chromosome is from bacteria or fungi. In some cases, size selection can be performed after each step in the method. In one embodiment, the molecular analysis comprises amplicon size selection on the amplicons generated from the PCR performed in step (a). Size selection can comprise any method known in the art for performing size selection such as, for example, column purification or isolation from an agarose gel. Size selection can comprise digestion and/or gel electrophoresis, optionally, wherein the electrophoresis is preceded by the digestion. In one embodiment, the amplicon size selection comprises digestion and/or gel electrophoresis of the amplicons, optionally, wherein the electrophoresis is preceded by the digestion. In some cases, size selection can be performed using SPRI beads or magnetic particles coated with carboxyl groups (in the form of succinic acid) that can bind DNA non-specifically and reversibly. Amplicon size selection can be employed to isolate amplicons sizes that are compatible with a sequencing platform or technology used for the molecular analysis. The amplicon size selection can isolate fragments that are at least 50 base pairs (bps), 75 bps, 100 bps, 125 bps, 150 bps, 175 bps, 200 bps, 225 bps, 250 bps, 275 bps, 300 bps, 325 bps, 350 bps, 375 bps, 400 bps, 425 bps, 450 bps, 475 bps, 500 bps, 550 bps, 600 bps, 650 bps, 700 bps, 750 bps, 800 bps, 850 bps, 900 bps, 950 bps or 1000 bps.

Also provided herein is a composition for use in a SG-seq enrichment method provided herein. The composition can comprise a first primer pair comprising a first primer comprising a sequence complementary to a common sequence present in a genetic edit at its 3′ end and a 5′ tail comprising a first universal sequence and a plurality of semi-guided primers comprising a priming sequence complementary to a variable locus-specific sequence at its 3′ end and a 5′ tail comprising a second universal sequence. The first primer and/or each second primer of the plurality of second primers can comprise sequencing primer binding sites in the 5′ tail. The first primer and/or each second primer of the plurality of second primers can comprise an adapter sequence compatible with a third generation sequencing platform (e.g., Oxford Nanopore Technologies MinION sequencing platform) in the 5′ tail. In some cases, the composition can further comprise a second primer pair comprising a first primer comprising a 3′ end comprising sequence complementary to the first universal sequence in the 5′ tail of the first primer from the first primer pair and a second primer comprising a 3′ end comprising sequence complementary to the second universal sequence in the 5′ tail of each of the second primers from the first primer pair. The first primer and/or the second primer from the second primer pair can comprise 5′ tails comprising non-complementary sequence that comprise sequencing primer binding sites. The first primer and/or the second primer from the second primer pair can comprise 5′ tails comprising non-complementary sequence that comprise an adapter sequence compatible with a third generation sequencing platform (e.g., Oxford Nanopore Technologies MinION sequencing platform) in the 5′ tail. In one embodiment, the composition further comprises one or more reagents for performing nucleic extraction, purification, PCR, size selection or sequencing.

In one embodiment, the priming sequence in the plurality of second primers for any SG-seq method or composition provided herein comprises a mixture of fully or partially random nucleotides and nucleotides that are complementary to the variable locus-specific sequence, thereby making the second primers semi-guided in nature. The priming sequence can comprise at least 1, at least 2, at least 3, at least 4, at least 5, at least 6, at least 7, at least 8, at least 9 or at least 10 nucleotides that are complementary to the variable locus-specific sequence. The priming sequence can comprise 1, 2, 3, 4, 5, 6, 7, 8, 9 or 10 nucleotides that are complementary to the variable locus-specific sequence. The priming sequence can comprise between 0-3, between 1-4, between 2-5, between 3-6, between 4-7, between 5-8, between 6-9, between 7-10 or between 8-11 nucleotides that are complementary to the variable locus-specific sequence. In one embodiment, the priming sequence comprises 3-5 nucleotides that are complementary to the variable locus-specific sequence.

In one embodiment, the variable locus-specific sequence is near the one or each genetic edit from the plurality of genetic edits. The variable locus-specific sequence can be present in the microbial strain at least once near the one or each genetic edit of the plurality of genetic edits. The variable locus-specific sequence can be less than 3 kilobases (kbs), less than 1.5 kb, less than 1 kb, less than 750 base-pairs (bps), less than 500 bps, less than 250 bps, less than 125 bps, less than 100 bps, less than 75 bps, less than 50 bps, less than 25 bps, less than 20 bps, less than 15 bps, less than 10 bps, or less than 5 bps away from the one or each of the plurality of genetic edits. In one embodiment, the variable locus-specific sequence is less than 1.5 kb away from the one or each of the plurality of genetic edits.

In one embodiment, the molecular analysis of the amplicons in the SG-seq methods provided herein comprises DNA sequencing using sequencing primers directed to the sequencing primer binding sites. The molecular analysis can comprises any first, second, or third generation DNA sequencing method known in the art and/or provided herein. In one embodiment, the molecular analysis further comprises comparing sequence reads obtained from the sequencing of the amplicons to a reference database for the microbial strain using a computer-implemented method, thereby identifying the one or the plurality of genetic edits. The computer-implemented method can utilize a sequence similarity search program, a sequence composition search program or a combination thereof. The sequence similarity search program can employ a basic local alignment search tool (BLAST) algorithm, fuzzy logic, lowest common ancestor (LCA) algorithm or a profile hidden Markov Model (pHMM). The sequence composition search program employs interpolated Markov models (IMMs), naive Bayesian classifiers, k-mers or k-means/k-nearest-neighbor algorithms. In one embodiment, the sequence composition search program employs k-mers. The k-mers can comprise short nucleotide sequences comprising nucleotide bases complementary to a sequence near the one or each of the plurality of genetic edits. In one embodiment, detection of the short nucleotide sequence in the sequence reads indicates presence of the one or each of the plurality of genetic edits in the microbial strain. The sequence near the one or each of the plurality of genetic edits is within 25 base pairs (bps), 20 bps, 15 bps, 10 bps, or 5 bps of the one or each of the plurality of genetic edits.

In one embodiment, the common sequence in at least one genetic edit in the plurality of genetic edits is different from the common sequence in each other genetic edit in the plurality of genetic edits. In another embodiment, the common sequence in each genetic edit in the plurality of genetic edits is different from the common sequence in each other genetic edit in the plurality of genetic edits. In yet another embodiment, the common sequence is specific to a genetic edit. In one embodiment, the common sequence is a portion of the sequence that makes up the genetic edit. The portion of the sequence that makes up the genetic edit that can serve as the common sequence can be at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95 or 100 nucleotides in length. The portion of the sequence that makes up the genetic edit that can serve as the common sequence can be at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95 or 100 nucleotides in length. The portion of the sequence that makes up the genetic edit that can serve as the common sequence can be between 1-5, between 5-10, between 10-15, between 15-20, between 20-25, between 25-30, between 30-35, between 35-40, between 40-45, between 45-50, between 50-55, between 55-60, between 60-65, between 65-70, between 70-75, between 75-80, between 80-85, between 85-90, between 90-95 or between 95-100 nucleotides in length. The portion of the sequence that makes up the genetic edit that can serve as the common sequence can be from 1-5, from 5-10, from 10-15, from 15-20, from 20-25, from 25-30, from 30-35, from 35-40, from 40-45, from 45-50, from 50-55, from 55-60, from 60-65, from 65-70, from 70-75, from 75-80, from 80-85, from 85-90, from 90-95 or from 95-100 nucleotides in length, inclusive of the endpoints. In another embodiment, the common sequence is sequence added to the genetic edit that does not alter or affect the function of the genetic edit. In some cases, the common sequence added to the genetic edit can be shared with at least one genetic edit in the plurality of genetic edits. In some cases, the common sequence added to the genetic edit can be shared with each of the genetic edits in the plurality of genetic edits. The common sequence added to the genetic edit can be at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95 or 100 nucleotides in length. The common sequence added to the genetic edit can be at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95 or 100 nucleotides in length. The common sequence added to the genetic edit can be between 1-5, between 5-10, between 10-15, between 15-20, between 20-25, between 25-30, between 30-35, between 35-40, between 40-45, between 45-50, between 50-55, between 55-60, between 60-65, between 65-70, between 70-75, between 75-80, between 80-85, between 85-90, between 90-95 or between 95-100 nucleotides in length. The common sequence added to the genetic edit can be from 1-5, from 5-10, from 10-15, from 15-20, from 20-25, from 25-30, from 30-35, from 35-40, from 40-45, from 45-50, from 50-55, from 55-60, from 60-65, from 65-70, from 70-75, from 75-80, from 80-85, from 85-90, from 90-95 or from 95-100 nucleotides in length, inclusive of the endpoints. The common sequence and/or genetic edit (of which the common sequence can be all or a part of) can be selected from any genetic element including a promoter sequence, a termination sequence, a degron sequence, a protein solubility tag sequence, a protein degradation tag sequence, a ribosomal binding site (RBS) sequence, a landing pad primer binding sequence, an antibiotic resistance gene sequence or any portion thereof. The common sequence and/or genetic edit (of which the common sequence can be all or a part of) can be an exogenous gene sequence or mutated version thereof. The common sequence and/or genetic edit (of which the common sequence can be all or a part of) can be a mutated version of a gene present in the genome of the organism. The mutated version of the gene sequence can contain or comprise a single nucleotide polymorphism (SNP). In one embodiment, the one genetic edit or the plurality of genetic edits introduced into an organism that is subsequently subjected to a SG-seq enrichment method provided herein can be derived from or introduced as a part of a library of genetic edits. The library of genetic edits can be libraries of promoter sequences, termination sequences, solubility tag sequences, degradation tag sequences or SNP sequences that can be generated using any of the methods described in WO 2020/092704, WO 2018/226900, WO 2018/226880 or WO 2017/100377, each of which is herein incorporated by reference in their entireties. Said libraries of promoter sequences, termination sequences, solubility tag sequences, degradation tag sequences or SNP sequences can be introduced using the promoter swapping, terminator (stop) swapping, solubility tag swapping, degradation tag swapping or SNP swapping methods described in WO 2018/226900, WO 2018/226880 or WO 2017/100377. In one embodiment, the common sequence is shared by all members of a library introduced or to be introduced into the genome of an organism (e.g., microbial strain). In one embodiment, the common sequence is shared by a subset of members of a library introduced or to be introduced into the genome of an organism (e.g., microbial strain). In one embodiment, the common sequence and/or genetic edit (of which the common sequence can be all or a part of) is not a transposon or transposon-related sequence.

### Gene Editing

As described herein, the enrichment methods provided herein (e.g., CS-seq and SG-seq) can be used to genotype an organism (e.g., microbial strain) that has been subjected to genetic engineering or gene editing. In one embodiment, an enrichment method provided herein (e.g., CS-seq or SG-seq) is used to identify one or a plurality of genetic edits introduced into the genome of a microbial strain. The genetic edit or edits can comprise control elements (e.g., promoters, terminators, solubility tags, degradation tags or degrons), modified forms of genes (e.g., genes with desired SNP(s)), antisense nucleic acids, and/or one or more genes that are part of a metabolic or biochemical pathway. The gene editing can entail editing the genome of the organism and/or a separate genetic element present in the organism such as, for example, a plasmid or cosmid. The gene editing method used to generate the organism to be genotyped using an enrichment method provided herein (e.g., CS-seq or SG-seq) can be any gene editing method or system known in the art and can be selected based on the organism for which gene editing is desired. Non-limiting examples of gene editing include homologous recombination, lambda red recombineering, CRISPR, TALENS, FOK-1 nuclease, viral or phage transduction, ZN finger, meganuclease or other endonucleases.

### Homologous Recombination

In one aspect provided herein, the gene editing method used to generate the organism (e.g., microbial strain) to be genotyped using an enrichment method provided herein (e.g., CS-seq or SG-seq) can entail use of a homologous recombination based method known in the art. The homologous recombination based method can be selected from single-crossover homologous recombination, double-crossover homologous recombination, or lambda red recombineering. In order to be used in a homologous recombination based method known in the art, the genetic edit or plurality of genetic edit can be generated or assembled using any method known in the art. In one embodiment, the genetic edit or pools of genetics edit are generated using the deterministic assembly methods described in US 2020-0131508, which is herein incorporated by reference in its entirety.

Loop-in/Loop-Out

In some embodiments, the gene editing method used to generate the organism (e.g., microbial strain) to be genotyped using an enrichment method provided herein (e.g., CS-seq or SG-seq) teaches methods of looping out selected regions of DNA from the host organisms. The looping out method can be as described in Nakashima et al. 2014 “Bacterial Cellular Engineering by Genome Editing and Gene Silencing.” Int. J. Mol. Sci. 15(2), 2773-2793. Looping out deletion techniques are known in the art, and are described in (Tear et al. 2014 “Excision of Unstable Artificial Gene-Specific inverted Repeats Mediates Scar-Free Gene Deletions in Escherichia coli.” Appl. Biochem. Biotech. 175:1858-1867). The looping out methods used can be performed using single-crossover homologous recombination or double-crossover homologous recombination. In one embodiment, looping out of selected regions can entail using single-crossover homologous recombination.

In one aspect provided herein, the gene editing method used to generate the organism (e.g., microbial strain) to be genotyped using an enrichment method provided herein (e.g., CS-seq or SG-seq) can entail the use of sets of proteins from one or more recombination systems. Said recombination systems can be endogenous to the microbial host cell or can be introduced heterologously. The sets of proteins of the one or more heterologous recombination systems can be introduced as nucleic acids (e.g., as plasmid, linear DNA or RNA, or integron) and be integrated into the genome of the host cell or be stably expressed from an extrachromosomal element. The sets of proteins of the one or more heterologous recombination systems can be introduced as RNA and be translated by the host cell. The sets of proteins of the one or more heterologous recombination systems can be introduced as proteins into the host cell. The sets of proteins of the one or more recombination systems can be from a lambda red recombination system, a RecET recombination system, a Red/ET recombination system, any homologs, orthologs or paralogs of proteins from a lambda red recombination system, a RecET recombination system, or Red/ET recombination system or any combination thereof. The recombination methods and/or sets of proteins from the RecET recombination system can be any of those as described in Zhang Y., Buchholz F., Muyrers J. P. P. and Stewart A. F. “A new logic for DNA engineering using recombination in E. coli.” Nature Genetics 20 (1998) 123-128; Muyrers, J. P. P., Zhang, Y., Testa, G., Stewart, A. F. “Rapid modification of bacterial artificial chromosomes by ET-recombination.” Nucleic Acids Res. 27 (1999) 1555-1557; Zhang Y., Muyrers J. P. P., Testa G. and Stewart A. F. “DNA cloning by homologous recombination in E. coli.” Nature Biotechnology 18 (2000) 1314-1317 and Muyrers J P et al., “Techniques: Recombinogenic engineering—new options for cloning and manipulating DNA” Trends Biochem Sci. 2001 May; 26(5):325-31, which are herein incorporated by reference. The sets of proteins from the Red/ET recombination system can be any of those as described in Rivero-Müller, Adolfo et al. “Assisted large fragment insertion by Red/ET-recombination (ALFIRE)—an alternative and enhanced method for large fragment recombineering” Nucleic acids research vol. 35, 10 (2007): e78, which is herein incorporated by reference.

Lambda RED Mediated Recombination

In one aspect provided herein, the gene editing method used to generate the organism (e.g., microbial strain) to be genotyped using an enrichment method provided herein (e.g., CS-seq or SG-seq) can entail the use of a set of proteins from the lambda red-mediated recombination system. The use of lambda red-mediated homologous recombination to generate the organism to be genotyped using an enrichment method provided herein (e.g., CS-seq or SG-seq) can be as described by Datsenko and Wanner, PNAS USA 97:6640-6645 (2000), the contents of which are hereby incorporated by reference in their entirety. The set of proteins from the lambda red recombination system can comprise the exo, beta or gam proteins or any combination thereof. Gam can prevent both the endogenous RecBCD and SbcCD nucleases from digesting linear DNA introduced into a microbial host cell, while exo is a 5′→3′ dsDNA-dependent exonuclease that can degrade linear dsDNA starting from the 5′ end and generate 2 possible products (i.e., a partially dsDNA duplex with single-stranded 3′ overhangs or a ssDNA whose entire complementary strand was degraded) and beta can protect the ssDNA created by Exo and promote its annealing to a complementary ssDNA target in the cell. Beta expression can be required for lambda red based recombination with an ssDNA oligo substrate as described at https://blog.addgene.org/lambda-red-a-homologous-recombination-based-technique-for-genetic-engineering, the contents of which are herein incorporated by reference.

In one embodiment, the gene editing method used to generate the organism to be genotyped using an enrichment method provided herein (e.g., CS-seq or SG-seq) is implemented in a microbial host cell that already stably expresses lambda red recombination genes such as the DY380 strain described at https://blog.addgene.org/lambda-red-a-homologous-recombination-based-technique-for-genetic-engineering, the contents of which are herein incorporated by reference. Other bacterial strains that comprise components of the lambda red recombination system and can be utilized to generate the organism to be genotyped using an enrichment method provided herein (e.g., CS-seq or SG-seq) can be found in Thomason et al (Recombineering: Genetic Engineering in Bacteria Using Homologous Recombination. Current Protocols in Molecular Biology. 106:V:1.16:1.16.1-1.16.39) and Sharan et al (Recombineering: A Homologous Recombination-Based Method of Genetic Engineering. Nature protocols. 2009; 4(2):206-223), the contents of each of which are herein incorporated by reference.

As provided herein, the set of proteins of the lambda red recombination system can be introduced into the microbial host cell prior to implementation of any of the editing methods known in the art and/or provided herein. Genes for each of the proteins of the lambda red recombination system can be introduced on nucleic acids (e.g., as plasmids, linear DNA or RNA, a mini-λ, a lambda red prophage or integrons) and be integrated into the genome of the host cell or expressed from an extrachromosomal element. In some cases, each of the components (i.e., exo, beta, gam or combinations thereof) of the lambda red recombination system can be introduced as an RNA and be translated by the host cell. In some cases, each of the components (i.e., exo, beta, gam or combinations thereof) of the lambda red recombination system can be introduced as a protein into the host cell.

In one embodiment, genes for the set of proteins of the lambda red recombination system are introduced on a plasmid. The set of proteins of the lambda red recombination system on the plasmid can be under the control of a promoter such as, for example, the endogenous phage pL promoter. In one embodiment, the set of proteins of the lambda red recombination system on the plasmid is under the control of an inducible promoter. The inducible promoter can be inducible by the addition or depletion of a reagent or by a change in temperature. In one embodiment, the set of proteins of the lambda red recombination system on the plasmid is under the control of an inducible promoter such as the IPTG-inducible lac promoter or the arabinose-inducible pBAD promoter. A plasmid expressing genes for the set of proteins of the lambda red recombination system can also express repressors associated with a specific promoter such as, for example, the lad, araC or cI857 repressors associated with the IPTG-inducible lac promoter, the arabinose-inducible pBAD promoter and the endogenous phage pL promoters, respectively.

In one embodiment, genes for the set of proteins of the lambda red recombination system are introduced on a mini-λ, which a defective non-replicating, circular piece of phage DNA, that when introduced into microbial host cell, integrates into the genome as described at https://blog.addgene.org/lambda-red-a-homologous-recombination-based-technique-for-genetic-engineering, the contents of which are herein incorporated by reference.

In one embodiment, genes for the set of proteins of the lambda red recombination system are introduced on a lambda red prophage, which can allow for stable integration of the lambda red recombination system into a microbial host cell such as described at https://blog.addgene.org/lambda-red-a-homologous-recombination-based-technique-for-genetic-engineering, the contents of which are herein incorporated by reference.

CRISPR Mediated Gene Editing

In one aspect provided herein, the gene editing method used to generate the organism (e.g., microbial strain) to be genotyped using an enrichment method provided herein (e.g., CS-seq or SG-seq) can entail the use of Clustered Regularly Interspaced Short Palindromic Repeat (CRISPR). As provided herein, the RNA-guided DNA endonucleases of the CRISPR/Cas system can be introduced into the microbial host cell prior to implementation of the method. The RNA-guided DNA endonucleases of the CRISPR/Cas system can be introduced as nucleic acids (e.g., as plasmid, linear DNA or RNA, or integron) and be integrated into the genome of the host cell or expressed from an extrachromosomal element. The RNA-guided DNA endonucleases of the CRISPR/Cas system can be introduced as an RNA and be translated by the host cell. The RNA-guided DNA endonucleases of the CRISPR/Cas system can be introduced as a protein into the host cell.

The CRISPR/Cas system is a prokaryotic immune system that confers resistance to foreign genetic elements such as those present within plasmids and phages and that provides a form of acquired immunity. CRISPR stands for Clustered Regularly Interspaced Short Palindromic Repeat, and cas stands for CRISPR-associated system, and refers to the small cas genes associated with the CRISPR complex.

CRISPR-Cas systems are most broadly characterized as either Class 1 or Class 2 systems. The main distinguishing feature between these two systems is the nature of the Cas-effector module. Class 1 systems require assembly of multiple Cas proteins in a complex (referred to as a “Cascade complex”) to mediate interference, while Class 2 systems use a large single Cas enzyme to mediate interference. Each of the Class 1 and Class 2 systems are further divided into multiple CRISPR-Cas types based on the presence of a specific Cas protein. For example, the Class 1 system is divided into the following three types: Type I systems, which contain the Cas3 protein; Type III systems, which contain the Cas10 protein; and the putative Type IV systems, which contain the Csf1 protein, a Cas8-like protein. Class 2 systems are generally less common than Class 1 systems and are further divided into the following three types: Type II systems, which contain the Cas9 protein; Type V systems, which contain Cas12a protein (previously known as Cpf1, and referred to as Cpf1 herein), Cas12b (previously known as C2c1), Cas12c (previously known as C2c3), Cas12d (previously known as CasY), and Cas12e (previously known as CasX); and Type VI systems, which contain Cas13a (previously known as C2c2), Cas13b, and Cas13c. Pyzocha et al., ACS Chemical Biology, Vol. 13 (2), pgs. 347-356. In one embodiment, the CRISPR-Cas system for use in the methods provided herein is a Class 2 system. In one embodiment, the CRISPR-Cas system for use in the methods provided herein is a Type II, Type V or Type VI Class 2 system. In one embodiment, the CRISPR-Cas system for use in the methods provided herein comprises a component selected from Cas9, Cas12a, Cas12b, Cas12c, Cas12d, Cas12e, Cas13a, Cas13b, Cas13c, and MAD7, or homologs, orthologs or paralogs thereof. In one embodiment, the CRISPR-Cas system for use in the methods provided herein comprises Cpf1, or homologs, orthologs or paralogs thereof. In one embodiment, the CRISPR-Cas system for use in the methods provided herein comprises MAD7, or homologs, orthologs or paralogs thereof.

CRISPR systems used in methods disclosed herein comprise a Cas effector module comprising one or more nucleic acid (e.g., RNA) guided CRISPR-associated (Cas) nucleases, referred to herein as Cas effector proteins. In some embodiments, the Cas proteins can comprise one or multiple nuclease domains. A Cas effector protein can target single stranded or double stranded nucleic acid molecules (e.g. DNA or RNA nucleic acids) and can generate double strand or single strand breaks. In some embodiments, the Cas effector proteins are wild-type or naturally occurring Cas proteins. In some embodiments, the Cas effector proteins are mutant Cas proteins, wherein one or more mutations, insertions, or deletions are made in a WT or naturally occurring Cas protein (e.g., a parental Cas protein) to produce a Cas protein with one or more altered characteristics compared to the parental Cas protein.

In some instances, the Cas protein is a wild-type (WT) nuclease. Non-limiting examples of suitable Cas proteins for use in the present disclosure include C2c1, C2c2, C2c3, Cas1, Cas1B, Cas2, Cas3, Cas4, Cas5, Cash, Cas7, Cas8, Cas9 (also known as Csn1 and Csx12), Cas10, Cpf1, Csy1, Csy2, Csy3, Cse1, Cse2, Csc1, Csc2, Csa5, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx100, Csx16, CsaX, Csx3, Csx1, Csx15, Csf1, Csf2, Csf3, Csf4, MAD1-20, SmCsm1, homologs thereof, orthologues thereof, variants thereof, mutants thereof, or modified versions thereof. Suitable nucleic acid guided nucleases (e.g., Cas9) can be from an organism from a genus, which includes but is not limited to: Thiomicrospira, Succinivibrio, Candidatus, Porphyromonas, Acidomonococcus, Prevotella, Smithella, Moraxella, Synergistes, Francisella, Leptospira, Catenibacterium, Kandleria, Clostridium, Dorea, Coprococcus, Enterococcus, Fructobacillus, Weissella, Pediococcus, Corynebacter, Sutterella, Legionella, Treponema, Roseburia, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, Mycoplasma, Alicyclobacillus, Brevibacilus, Bacillus, Bacteroidetes, Brevibacilus, Carnobacterium, Clostridiaridium, Clostridium, Desulfonatronum, Desulfovibrio, Helcococcus, Leptotrichia, Listeria, Methanomethyophilus, Methylobacterium, Opitutaceae, Paludibacter, Rhodobacter, Sphaerochaeta, Tuberibacillus, and Campylobacter. Species of organism of such a genus can be as otherwise herein discussed.

Suitable nucleic acid guided nucleases (e.g., Cas9) can be from an organism from a phylum, which includes but is not limited to Firmicute, Actinobacteria, Bacteroidetes, Proteobacteria, Spirochates, and Tenericutes. Suitable nucleic acid guided nucleases can be from an organism from a class, which includes but is not limited to Erysipelotrichia, Clostridia, Bacilli, Actinobacteria, Bacteroidetes, Flavobacteria, Alphaproteobacteria, Betaproteobacteria, Gammaproteobacteria, Deltaproteobacteria, Epsilonproteobacteria, Spirochaetes, and Mollicutes. Suitable nucleic acid guided nucleases can be from an organism from an order, which includes but is not limited to Clostridiales, Lactobacillales, Actinomycetales, Bacteroidales, Flavobacteriales, Rhizobiales, Rhodospirillales, Burkholderiales, Neisseriales, Legionellales, Nautiliales, Campylobacterales, Spirochaetales, Mycoplasmatales, and Thiotrichales. Suitable nucleic acid guided nucleases can be from an organism from within a family, which includes but is not limited to: Lachnospiraceae, Enterococcaceae, Leuconostocaceae, Lactobacillaceae, Streptococcaceae, Peptostreptococcaceae, Staphylococcaceae, Eubacteriaceae, Corynebacterineae, Bacteroidaceae, Flavobacterium, Cryomoorphaceae, Rhodobiaceae, Rhodospirillaceae, Acetobacteraceae, Sutterellaceae, Neisseriaceae, Legionellaceae, Nautiliaceae, Campylobacteraceae, Spirochaetaceae, Mycoplasmataceae, and Francisellaceae.

Other nucleic acid guided nucleases (e.g., Cas9) suitable for use in the methods, systems, and compositions of the present disclosure include those derived from an organism such as, but not limited to: Thiomicrospira sp. XS5, Eubacterium rectale, Succinivibrio dextrinosolvens, Candidatus Methanoplasma termitum, Candidatus Methanomethylophilus alvus, Porphyromonas crevioricanis, Flavobacterium branchiophilum, Acidomonococcus sp., Lachnospiraceae bacterium COE1, Prevotella brevis ATCC 19188, Smithella sp. SCADC, Moraxella bovoculi, Synergistes jonesii, Bacteroidetes oral taxon 274, Francisella tularensis, Leptospira inadai serovar Lyme str. 10, Acidomonococcus sp. crystal structure (5B43) S. mutans, S. agalactiae, S. equisimilis, S. sanguinis, S. pneumonia; C. jejuni, C. coli; N. salsuginis, N. tergarcus; S. auricularis, S. carnosus; N. meningitides, N. gonorrhoeae; L. monocytogenes, L. ivanovii; C. botulinum, C. difficile, C. tetani, C. sordellii; Francisella tularensis l, Prevotella albensis, Lachnospiraceae bacterium MC2017 1, Butyrivibrio proteoclasticus, Peregrinibacteria bacterium GW2011_GWA2_33_10, Parcubacteria bacterium GW2011_GWC2_44_17, Smithella sp. SCADC, Microgenomates, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens, Porphyromonas macacae, Catenibacterium sp. CAG:290, Kandleria vitulina, Clostridiales bacterium KA00274, Lachnospiraceae bacterium 3-2, Dorea longicatena, Coprococcus catus GD/7, Enterococcus columbae DSM 7374, Fructobacillus sp. EFB-N1, Weissella halotolerans, Pediococcus acidilactici, Lactobacillus curvatus, Streptococcus pyogenes, Lactobacillus versmoldensis, and Filifactor alocis ATCC 35896. See, U.S. Pat. Nos. 8,697,359; 8,771,945; 8,795,965; 8,865,406; 8,871,445; 8,889,356; 8,895,308; 8,906,616; 8,932,814; 8,945,839; 8,993,233; 8,999,641; 9,822,372; 9,840,713; U.S. patent application Ser. No. 13/842,859 (US 2014/0068797 A1); U.S. Pat. Nos. 9,260,723; 9,023,649; 9,834,791; 9,637,739; U.S. patent application Ser. No. 14/683,443 (US 2015/0240261 A1); U.S. patent application Ser. No. 14/743,764 (US 2015/0291961 A1); U.S. Pat. Nos. 9,790,490; 9,688,972; 9,580,701; 9,745,562; 9,816,081; 9,677,090; 9,738,687; U.S. application Ser. No. 15/632,222 (US 2017/0369879 A1); U.S. application Ser. No. 15/631,989; U.S. application Ser. No. 15/632,001; and U.S. Pat. No. 9,896,696, each of which is herein incorporated by reference.

In some embodiments, a Cas effector protein comprises one or more of the following activities:

a nickase activity, i.e., the ability to cleave a single strand of a nucleic acid molecule;

a double stranded nuclease activity, i.e., the ability to cleave both strands of a double stranded nucleic acid and create a double stranded break;

an endonuclease activity;

an exonuclease activity; and/or

a helicase activity, i.e., the ability to unwind the helical structure of a double stranded nucleic acid.

In aspects of the disclosure the term “guide nucleic acid” refers to a polynucleotide comprising 1) a guide sequence capable of hybridizing to a target sequence (referred to herein as a “targeting segment”) and 2) a scaffold sequence capable of interacting with (either alone or in combination with a tracrRNA molecule) a nucleic acid guided nuclease as described herein (referred to herein as a “scaffold segment”). A guide nucleic acid can be DNA. A guide nucleic acid can be RNA. A guide nucleic acid can comprise both DNA and RNA. A guide nucleic acid can comprise modified non-naturally occurring nucleotides. In cases where the guide nucleic acid comprises RNA, the RNA guide nucleic acid can be encoded by a DNA sequence on a polynucleotide molecule such as a plasmid, linear construct generated using the methods and compositions provided herein.

In some embodiments, the guide nucleic acids described herein are RNA guide nucleic acids (“guide RNAs” or “gRNAs”) and comprise a targeting segment and a scaffold segment. In some embodiments, the scaffold segment of a gRNA is comprised in one RNA molecule and the targeting segment is comprised in another separate RNA molecule. Such embodiments are referred to herein as “double-molecule gRNAs” or “two-molecule gRNA” or “dual gRNAs.” In some embodiments, the gRNA is a single RNA molecule and is referred to herein as a “single-guide RNA” or an “sgRNA.” The term “guide RNA” or “gRNA” is inclusive, referring both to two-molecule guide RNAs and sgRNAs.

The DNA-targeting segment of a gRNA comprises a nucleotide sequence that is complementary or homologous to a sequence in a target nucleic acid sequence. The target nucleic acid sequence can be a locus in a genetic element such as a genome or plasmid. As such, the targeting segment of a gRNA interacts with a target nucleic acid in a sequence-specific manner via hybridization (i.e., base pairing), and the nucleotide sequence of the targeting segment determines the location within the target DNA that the gRNA will bind. The degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences. In some embodiments, a guide sequence is about or more than about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 75, or more nucleotides in length. In some embodiments, a guide sequence is less than about 75, 50, 45, 40, 35, 30, 25, 20 nucleotides in length. In aspects, the guide sequence is 10-30 nucleotides long. The guide sequence can be 15-20 nucleotides in length. The guide sequence can be 15 nucleotides in length. The guide sequence can be 16 nucleotides in length. The guide sequence can be 17 nucleotides in length. The guide sequence can be 18 nucleotides in length. The guide sequence can be 19 nucleotides in length. The guide sequence can be 20 nucleotides in length.

The scaffold segment of a guide RNA interacts with a one or more Cas effector proteins to form a ribonucleoprotein complex (referred to herein as a CRISPR-RNP or a RNP-complex). The guide RNA directs the bound polypeptide to a specific nucleotide sequence within a target nucleic acid sequence via the above-described targeting segment. The scaffold segment of a guide RNA comprises two stretches of nucleotides that are complementary to one another and which form a double stranded RNA duplex. Sufficient sequence within the scaffold sequence to promote formation of a targetable nuclease complex may include a degree of complementarity along the length of two sequence regions within the scaffold sequence, such as one or two sequence regions involved in forming a secondary structure. In some cases, the one or two sequence regions are comprised or present on the same polynucleotide. In some cases, the one or two sequence regions are comprised or present on separate polynucleotides. Optimal alignment may be determined by any suitable alignment algorithm, and may further account for secondary structures, such as self-complementarity within either the one or two sequence regions. In some embodiments, the degree of complementarity between the one or two sequence regions along the length of the shorter of the two when optimally aligned is about or more than about 25%, 30%, 40%, 50%, 60%, 70%, 80%, 90%, 95%, 97.5%, 99%, or higher. In some embodiments, at least one of the two sequence regions is about or more than about 5, 6, 7, 8, 9, 10, 11, 12, 13, 14, 15, 16, 17, 18, 19, 20, 25, 30, 40, 50, or more nucleotides in length.

A scaffold sequence of a subject gRNA can comprise a secondary structure. A secondary structure can comprise a pseudoknot region or stem-loop structure. In some examples, the compatibility of a guide nucleic acid and nucleic acid guided nuclease is at least partially determined by sequence within or adjacent to the secondary structure region of the guide RNA. In some cases, binding kinetics of a guide nucleic acid to a nucleic acid guided nuclease is determined in part by secondary structures within the scaffold sequence. In some cases, binding kinetics of a guide nucleic acid to a nucleic acid guided nuclease is determined in part by nucleic acid sequence with the scaffold sequence.

A compatible scaffold sequence for a gRNA-Cas effector protein combination can be found by scanning sequences adjacent to a native Cas nuclease loci. In other words, native Cas nucleases can be encoded on a genome within proximity to a corresponding compatible guide nucleic acid or scaffold sequence.

Nucleic acid guided nucleases can be compatible with guide nucleic acids that are not found within the nucleases endogenous host. Such orthogonal guide nucleic acids can be determined by empirical testing. Orthogonal guide nucleic acids can come from different bacterial species or be synthetic or otherwise engineered to be non-naturally occurring. Orthogonal guide nucleic acids that are compatible with a common nucleic acid-guided nuclease can comprise one or more common features. Common features can include sequence outside a pseudoknot region. Common features can include a pseudoknot region. Common features can include a primary sequence or secondary structure.

A guide nucleic acid can be engineered to target a desired target sequence by altering the guide sequence such that the guide sequence is complementary or homologous to the target sequence, thereby allowing hybridization between the guide sequence and the target sequence. A guide nucleic acid with an engineered guide sequence can be referred to as an engineered guide nucleic acid. Engineered guide nucleic acids are often non-naturally occurring and are not found in nature.

In one embodiment, the repair fragments comprising one or more genetic edits as provided herein that are introduced in each round any method provided herein serve as donor DNA and each genetic edit on each repair fragment is paired with a gRNA. Each gRNA can comprise sequence targeting a specific sequence at a locus in a genetic element (e.g., chromosome or plasmid) within the host cell. The donor DNA sequence can be used in combination with its paired guide RNA (gRNA) in a CRISPR method of gene editing using homology directed repair (HDR). The CRISPR complex can result in the strand breaks within the target gene(s) that can be repaired by using homology directed repair (HDR). HDR mediated repair can be facilitated by co-transforming the host cell with a donor DNA sequence generated using the methods and compositions provided herein. The donor DNA sequence can comprise a desired genetic perturbation (e.g., deletion, insertion (e.g., promoter, terminator, solubility or degradation tag), and/or single nucleotide polymorphism) as well as targeting sequences or homology arms that comprise sequence complementary or homologous to the sequence or locus targeted by the gRNA. In this embodiment, the CRISPR complex cleaves the target gene specified by the one or more gRNAs. The donor DNA sequence can then be used as a template for the homologous recombination machinery to incorporate the desired genetic perturbation into the host cell. The donor DNA can be single-stranded, double-stranded or a double-stranded plasmid. The donor DNA can lack a PAM sequence or comprise a scrambled, altered or non-functional PAM in order to prevent re-cleavage. In some cases, the donor DNA can contain a functional or non-altered PAM site. The mutated or edited sequence in the donor DNA (also flanked by the regions of homology) prevents re-cleavage by the CRISPR-complex after the mutation(s) has/have been incorporated into the genome. In some embodiments, homologous recombination is facilitated through the use or expression of sets of proteins from one or more recombination systems either endogenous to the host cell or introduced heterologously.

In one embodiment, the genetic edits found in the genome of an organism that can be genotyped using the enrichment methods provided herein (e.g., CS-seq or SG-seq) can be introduced singly or in pools using the methods described in US 2020-0283802, which is herein incorporated by reference in its entirety. In one embodiment, the single genetic edit or pools of genetic edits can be introduced into the genome of an organism that can be genotyped using the enrichment methods provided herein (e.g., CS-seq or SG-seq) in an iterative manner such as, for example, using the iterative editing methods described in US 2020-0283780, which is herein incorporated by reference in its entirety. The genetic edits can comprise control elements (e.g., promoters, terminators, solubility tags, degradation tags or degrons), modified forms of genes (e.g., genes with desired SNP(s)), antisense nucleic acids, and/or one or more genes that are part of a metabolic or biochemical pathway. In one embodiment, the genetic edit entails one or more deletions, for example, to inactivate a single gene or a plurality of genes. The gene editing can entail editing the genome of the host cell and/or a separate genetic element present in the host cell such as, for example, a plasmid or cosmid.

In some embodiments, the plurality of genetic edits found in the genome of an organism that can be genotyped using the enrichment methods provided herein (e.g., CS-seq or SG-seq) edits were introduced into the microbial strain by a iterative editing method.

In one embodiment, the genetic edits found in the genome of an organism that can be genotyped using the enrichment methods provided herein (e.g., CS-seq or SG-seq) were introduced into the microbial strain by an iterative editing method, wherein the iterative method comprises: (a) introducing into a microbial host cell a first plasmid comprising a first repair fragment and a selection marker gene, wherein the microbial host cell comprises a site-specific restriction enzyme or a sequence encoding a site-specific restriction enzyme is introduced into the microbial host cell along with the first plasmid, wherein the site-specific restriction enzyme targets a first locus in the microbial host cell, and wherein the first repair fragment comprises homology arms separated by a sequence for a genetic edit comprising a common sequence in or adjacent to a first locus in the microbial host cell, wherein the homology arms comprise sequence homologous to sequence that flanks the first locus in the microbial host cell; (b) growing the microbial host cells from step (a) in a medium selective for microbial host cells expressing the selection marker gene and isolating microbial host cells from cultures derived therefrom; (c) growing the microbial host cells isolated in step (b) in media not selective for the selection marker gene and isolating microbial host cells from cultures derived therefrom; and (d) repeating steps (a)-(c) in one or more additional rounds in the microbial host cells isolated in step (c), wherein each of the one or more additional rounds comprises introducing an additional plasmid comprising an additional repair fragment, wherein the additional repair fragment comprises homology arms separated by a sequence for a genetic edit comprising a common sequence in or adjacent to a locus in the microbial host cell, wherein the homology arms comprise sequence homologous to sequence that flanks the locus in the microbial host cell, wherein the additional plasmid comprises a different selection marker gene than the selection marker gene introduced in a previous round of selection, and wherein the microbial host cell comprises a site-specific restriction enzyme or a sequence encoding a site-specific restriction enzyme is introduced into the microbial host cell along with the additional plasmid that targets the first locus or another locus in the microbial host cell, thereby iteratively editing the microbial host cell to generate the microbial strain comprising the plurality of genetic edits; wherein a counterselection is not performed after at least one round of editing.

In one embodiment, the genetic edits found in the genome of an organism that can be genotyped using the enrichment methods provided herein (e.g., CS-seq or SG-seq) were introduced into the microbial strain by an iterative editing method, wherein the iterative method comprises: (a) introducing into the microbial host cell a first plasmid, a first guide RNA (gRNA) and a first repair fragment, wherein the gRNA comprises a sequence complementary to a first locus in the microbial host cell, wherein the first repair fragment comprises homology arms separated by a sequence for a genetic edit comprising a common sequence in or adjacent to a first locus in the microbial host cell, wherein the homology arms comprise sequence homologous to sequence that flanks the first locus in the microbial host cell, wherein the first plasmid comprises a selection marker gene and at least one or both of the gRNA and the repair fragment, and wherein: (i) the microbial host cell comprises an RNA-guided DNA endonuclease; or (ii) an RNA-guided DNA endonuclease is introduced into the microbial host cell along with the first plasmid; (b) growing the microbial host cells from step (a) in a medium selective for microbial host cells expressing the selection marker gene and isolating microbial host cells from cultures derived therefrom; (c) growing the microbial host cells isolated in step (b) in media not selective for the selection marker gene and isolating microbial host cells from cultures derived therefrom; and (d) repeating steps (a)-(c) in one or more additional rounds in the microbial host cells isolated in step (c), wherein each of the one or more additional rounds comprises introducing an additional plasmid, an additional gRNA and an additional repair fragment, wherein the additional gRNA comprises sequence complementary to a locus in the microbial host cell, wherein the additional repair fragment homology arms separated by a sequence for a genetic edit comprising a common sequence in or adjacent to a locus in the microbial host cell, wherein the homology arms comprise sequence homologous to sequence that flanks the locus in the microbial host cell, wherein the additional plasmid comprises a different selection marker gene than the selection marker gene introduced in a previous round of selection, and wherein the additional plasmid comprises at least one or both of the additional gRNA and the additional repair fragment, thereby iteratively editing the microbial host cell to generate the microbial strain comprising the plurality of genetic edits; wherein a counterselection is not performed after at least one round of editing.

In one embodiment, the genetic edits found in the genome of an organism that can be genotyped using the enrichment methods provided herein (e.g., CS-seq or SG-seq) were introduced into the microbial strain by an iterative editing method, wherein the iterative method comprises: (a) introducing into the microbial host cell a first plasmid comprising a first repair fragment and a selection marker gene, wherein the first repair fragment comprises homology arms separated by a sequence for a genetic edit comprising a common sequence in or adjacent to a first locus in the microbial host cell, wherein the homology arms comprise sequence homologous to sequence that flanks the first locus in the microbial host cell; (b) growing the microbial host cells from step (a) in a medium selective for microbial host cells expressing the selection marker gene and isolating microbial host cells from cultures derived therefrom; (c) growing the microbial host cells isolated in step (b) in media not selective for the selection marker gene and isolating microbial host cells from cultures derived therefrom; and (d) repeating steps (a)-(c) in one or more additional rounds in the microbial host cells isolated in step (c), wherein each of the one or more additional rounds comprises introducing an additional plasmid comprising an additional repair fragment, wherein the additional repair fragment comprises homology arms separated by sequence for a genetic edit comprising a common sequence in or adjacent to a locus in the microbial host cell, wherein the homology arms comprise sequence homologous to sequence that flanks the locus in the microbial host cell, and wherein the additional plasmid comprises a different selection marker gene than the selection marker gene introduced in a previous round of selection, thereby iteratively editing the microbial host cell to generate the microbial strain comprising the plurality of genetic edits; wherein a counterselection is not performed after at least one round of editing.

In some embodiments, the plurality of genetic edits found in the genome of an organism that can be genotyped using the enrichment methods provided herein (e.g., CS-seq or SG-seq) edits were introduced into the microbial strain by a pooled editing method.

In one embodiment, the plurality of genetic edits found in the genome of an organism that can be genotyped using the enrichment methods provided herein (e.g., CS-seq or SG-seq) edits were introduced into the microbial strain by a pooled editing method, wherein the pooled method comprises: (a) combining a base population of microbial host cells with a first pool of editing plasmids, wherein each editing plasmid in the pool comprises at least one repair fragment, and wherein the pool of editing plasmids comprises at least two different repair fragments, wherein each editing plasmid in the pool further comprises a selection marker gene, and wherein each repair fragment comprises sequence for one or more genetic edits comprising a common sequence in or adjacent to one or more target loci in the microbial host cells, and wherein sequence for each of the one or more genetic edits lies between homology arms, wherein the homology arms comprise sequence homologous to sequence that flanks a target loci from the one or more target loci in the microbial host cells; (b) introducing into individual microbial host cells from step (a) a plasmid or plasmids from the pool of editing plasmids; and (c) growing the microbial host cells from step (b) in a medium selective for microbial host cells expressing the selection marker gene and isolating microbial host cells from cultures derived therefrom, thereby generating the microbial strain comprising the plurality of genetic edits.

In one embodiment, the plurality of genetic edits found in the genome of an organism that can be genotyped using the enrichment methods provided herein (e.g., CS-seq or SG-seq) were introduced into the microbial strain by a pooled editing method, wherein the pooled method comprises: (a) combining a base population of microbial host cells with a first pool of editing plasmids, wherein each editing plasmid in the pool comprises at least one repair fragment, wherein the pool of editing plasmids comprises at least two different repair fragments, wherein each editing plasmid in the pool of editing plasmids further comprises a selection marker gene, and wherein the microbial host cells comprise one or more site-specific restriction enzymes or one or more sequences encoding one or more site-specific restriction enzymes is/are introduced into the microbial host cells along with the first pool of editing plasmids, wherein the one or more site-specific restriction enzymes target one or more target loci in the microbial host cells, wherein each repair fragment comprises sequence for one or more genetic edits comprising a common sequence in or adjacent to one or more target loci targeted by the one or more site-specific restriction enzymes, and wherein sequence for each of the one or more genetic edits lies between homology arms, wherein the homology arms comprise sequence homologous to sequence that flanks a target loci from the one or more target loci in the microbial host cell; (b) introducing into individual microbial host cells from step (a) a plasmid or plasmids from the pool of editing plasmids; and (c) growing the microbial host cells from step (b) in a medium selective for microbial host cells expressing the selection marker gene and isolating microbial host cells from cultures derived therefrom, thereby generating the microbial strain comprising the plurality of genetic edits.

In one embodiment, the genetic edits found in the genome of an organism that can be genotyped using the enrichment methods provided herein (e.g., CS-seq or SG-seq) were introduced into the microbial strain by a pooled editing method, wherein the pooled method comprises: (a) combining a base population of microbial host cells with a first pool of editing constructs comprising one or more editing plasmids, wherein each editing plasmid in the first pool of editing constructs comprises a selection marker gene and one or both of a guide RNA (gRNA) and a repair fragment, wherein the microbial host cells comprise an RNA-guided DNA endonuclease or an RNA-guided DNA endonuclease is introduced into the microbial host cells along with the first pool of editing constructs, and wherein the first pool of editing constructs comprise: (i) gRNAs that target the same target locus or loci, and at least two different repair fragments, wherein each repair fragment comprises a sequence for one or more genetic edits comprising a common sequence in or adjacent to the target locus, and wherein sequence for each of the genetic edits lies between homology arms, wherein the homology arms comprise sequence homologous to sequence that flanks the target locus in the microbial host cell; (ii) gRNAs that target at least two different target loci, and at least two different repair fragments, wherein each repair fragment comprises a sequence for the same one or more genetic edits comprising a common sequence in or adjacent to the target loci, and wherein sequence for each of the genetic edits lies between homology arms, wherein the homology arms comprise sequence homologous to sequence that flanks the target loci in the microbial host cell; or (iii) gRNAs that target at least two different target loci, and at least two different repair fragments, wherein each repair fragment comprises a sequence for one or more genetic edits comprising a common sequence in or adjacent to the target loci, and wherein sequence for each of the genetic edits lies between homology arms, wherein the homology arms comprise sequence homologous to sequence that flanks the target loci in the microbial host cell; (b) introducing into individual microbial host cells from step (a) the first pool of editing constructs comprising the one or more editing plasmids, wherein the first pool of editing constructs comprise gRNAs and repair fragments according to any one of step (a)(i)-(iii); and (c) growing the microbial host cells from step (b) in a medium selective for microbial host cells expressing the selection marker gene and isolating microbial host cells from cultures derived therefrom, thereby generating the microbial strain comprising the plurality of genetic edits.

In one embodiment, the genetic edits found in the genome of an organism that can be genotyped using the enrichment methods provided herein (e.g., CS-seq or SG-seq) were introduced into the microbial strain by a pooled editing method, wherein the pooled method comprises: (a) combining a base population of microbial host cells with a first pool of editing constructs comprising one or more editing plasmids, wherein each editing plasmid in the first pool of editing constructs comprises a selection marker gene and one or both of a guide RNA (gRNA) and a repair fragment, and wherein the first pool of editing constructs comprise: (i) gRNAs that target the same target locus or loci, and at least two different repair fragments, wherein each repair fragment comprises a sequence for one or more genetic edits comprising a common sequence in or adjacent to the target locus, and wherein sequence for each of the genetic edits lies between homology arms, wherein the homology arms comprise sequence homologous to sequence that flanks the target locus in the microbial host cell; (ii) gRNAs that target at least two different target loci, and at least two different repair fragments, wherein each repair fragment comprises a sequence for the same one or more genetic edits comprising a common sequence in or adjacent to the target loci, and wherein sequence for each of the genetic edits lies between homology arms, wherein the homology arms comprise sequence homologous to sequence that flanks the target loci in the microbial host cell; or (iii) gRNAs that target at least two different target loci, and at least two different repair fragments, wherein each repair fragment comprises a sequence for one or more genetic edits comprising a common sequence in or adjacent to the target loci, and wherein sequence for each of the genetic edits lies between homology arms, wherein the homology arms comprise sequence homologous to sequence that flanks the target loci in the microbial host cell; (b) introducing into individual microbial host cells from step (a) an RNA-guided DNA endonuclease and the first pool of editing constructs comprising the one or more editing plasmids, wherein the first pool of editing constructs comprise gRNAs and repair fragments according to any one of step (a)(i)-(iii); and (c) growing the microbial host cells from step (b) in a medium selective for microbial host cells expressing the selection marker gene and isolating microbial host cells from cultures derived therefrom, thereby generating the microbial strain comprising the plurality of genetic edits.

In some embodiments, the present disclosure provides a gRNA complexed with a site-directed modifying polypeptide to form an RNP-complex that is capable of being directly introduced into a host cell comprising a target locus for which the targeting segment of the gRNA comprising sequence that is complementary thereto. The site-directed modifying polypeptide can be a nucleic acid guided nuclease. The nucleic acid guided nuclease can be any nucleic acid guided nuclease as known in the art and/or provided herein (e.g., Cas9). The nucleic acid guided nuclease can be guided by and RNA (e.g., gRNA) and thus be referred to as an RNA guided nuclease or RNA guided endonuclease.

### Organisms Amenable to Enrichment

The disclosed targeted genome enrichment methods provided herein (e.g., CS-seq or SG-seq) are applicable to any host cell organism where desired traits can be identified in a population of genetic mutants, such as, for example, industrial microbial cell cultures (e.g., Corynebacterium and A. niger).

Thus, as used herein, the terms “microorganism” or “microbe” should be taken broadly. It includes, but is not limited to, the two prokaryotic domains, Bacteria and Archaea, as well as certain eukaryotic fungi and protists. However, in certain aspects, “higher” eukaryotic organisms such as insects, plants, and animals can be utilized in the methods taught herein.

Suitable host cells include, but are not limited to: bacterial cells, algal cells, plant cells, fungal cells, insect cells, and mammalian cells. In one illustrative embodiment, suitable host cells include E. coli (e.g., SHuffle™ competent E. coli available from New England BioLabs in Ipswich, Mass.).

Other suitable host organisms of the present disclosure include microorganisms of the genus Corynebacterium. In some embodiments, preferred Corynebacterium strains/species include: C. efficiens, with the deposited type strain being DSM44549, C. glutamicum, with the deposited type strain being ATCC13032, and C. ammoniagenes, with the deposited type strain being ATCC6871. In some embodiments the preferred host of the present disclosure is C. glutamicum.

Suitable host strains of the genus Corynebacterium, in particular of the species Corynebacterium glutamicum, are in particular the known wild-type strains: Corynebacterium glutamicum ATCC13032, Corynebacterium acetoglutamicum ATCC15806, Corynebacterium acetoacidophilum ATCC13870, Corynebacterium melassecola ATCC17965, Corynebacterium thermoaminogenes FERM BP-1539, Brevibacterium flavum ATCC14067, Brevibacterium lactofermentum ATCC13869, and Brevibacterium divaricatum ATCC14020; and L-amino acid-producing mutants, or strains, prepared therefrom, such as, for example, the L-lysine-producing strains: Corynebacterium glutamicum FERM-P 1709, Brevibacterium flavum FERM-P 1708, Brevibacterium lactofermentum FERM-P 1712, Corynebacterium glutamicum FERM-P 6463, Corynebacterium glutamicum FERM-P 6464, Corynebacterium glutamicum DM58-1, Corynebacterium glutamicum DG52-5, Corynebacterium glutamicum DSM5714, and Corynebacterium glutamicum DSM12866.

The term “Micrococcus glutamicus” has also been in use for C. glutamicum. Some representatives of the species C. efficiens have also been referred to as C. thermoaminogenes in the prior art, such as the strain FERM BP-1539, for example.

In some embodiments, the host cell of the present disclosure is a eukaryotic cell.

Suitable eukaryotic host cells include, but are not limited to: fungal cells, algal cells, insect cells, animal cells, and plant cells. Suitable fungal host cells include, but are not limited to: Ascomycota, Basidiomycota, Deuteromycota, Zygomycota, Fungi imperfecti. Certain preferred fungal host cells include yeast cells and filamentous fungal cells. Suitable filamentous fungi host cells include, for example, any filamentous forms of the subdivision Eumycotina and Oomycota. (see, e.g., Hawksworth et al., In Ainsworth and Bisby's Dictionary of The Fungi, 8th edition, 1995, CAB International, University Press, Cambridge, UK, which is incorporated herein by reference). Filamentous fungi are characterized by a vegetative mycelium with a cell wall composed of chitin, cellulose and other complex polysaccharides. The filamentous fungi host cells are morphologically distinct from yeast.

In certain illustrative, but non-limiting embodiments, the filamentous fungal host cell may be a cell of a species of: Achlya, Acremonium, Aspergillus, Aureobasidium, Bjerkandera, Ceriporiopsis, Cephalosporium, Chrysosporium, Cochliobolus, Corynascus, Cryphonectria, Cryptococcus, Coprinus, Coriolus, Diplodia, Endothis, Fusarium, Gibberella, Gliocladium, Humicola, Hypocrea, Myceliophthora (e.g., Myceliophthora thermophila), Mucor, Neurospora, Penicillium, Podospora, Phlebia, Piromyces, Pyricularia, Rhizomucor, Rhizopus, Schizophyllum, Scytalidium, Sporotrichum, Talaromyces, Thermoascus, Thielavia, Tramates, Tolypocladium, Trichoderma, Verticillium, Volvariella, or teleomorphs, or anamorphs, and synonyms or taxonomic equivalents thereof. In one embodiment, the filamentous fungus is selected from the group consisting of A. nidulans, A. oryzae, A. sojae, and Aspergilli of the A. niger group. In an embodiment, the filamentous fungus is Aspergillus niger.

In another embodiment, specific mutants of the fungal species are used for the methods and systems provided herein. In one embodiment, specific mutants of the fungal species are used which are suitable for the high-throughput and/or automated methods and systems provided herein. Examples of such mutants can be strains that protoplast very well; strains that produce mainly or, more preferably, only protoplasts with a single nucleus; strains that regenerate efficiently in microtiter plates, strains that regenerate faster and/or strains that take up polynucleotide (e.g., DNA) molecules efficiently, strains that produce cultures of low viscosity such as, for example, cells that produce hyphae in culture that are not so entangled as to prevent isolation of single clones and/or raise the viscosity of the culture, strains that have reduced random integration (e.g., disabled non-homologous end joining pathway) or combinations thereof.

In yet another embodiment, a specific mutant strain for use in the methods and systems provided herein can be strains lacking a selectable marker gene such as, for example, uridine-requiring mutant strains. These mutant strains can be either deficient in orotidine 5 phosphate decarboxylase (OMPD) or orotate p-ribosyl transferase (OPRT) encoded by the pyrG or pyrE gene, respectively (T. Goosen et al., Curr Genet. 1987, 11:499 503; J. Begueret et al., Gene. 1984 32:487 92.

In one embodiment, specific mutant strains for use in the methods and systems provided herein are strains that possess a compact cellular morphology characterized by shorter hyphae and a more yeast-like appearance. In one embodiment, the specific filamentous fungus for use in the methods provided comprise a non-mycelium, pellet-like morphology due to a genetic perturbation in one or more genes that affect filamentous fungal cell morphology as described in PCT/US2019/035793, which is herein incorporated by reference in its entirety.

Suitable yeast host cells include, but are not limited to: Candida, Hansenula, Saccharomyces, Schizosaccharomyces, Pichia, Kluyveromyces, and Yarrowia. In some embodiments, the yeast cell is Hansenula polymorpha, Saccharomyces cerevisiae, Saccaromyces carlsbergensis, Saccharomyces diastaticus, Saccharomyces norbensis, Saccharomyces kluyveri, Schizosaccharomyces pombe, Pichia pastoris, Pichia finlandica, Pichia trehalophila, Pichia kodamae, Pichia membranaefaciens, Pichia opuntiae, Pichia thermotolerans, Pichia salictaria, Pichia quercuum, Pichia pijperi, Pichia stipitis, Pichia methanolica, Pichia angusta, Kluyveromyces lactis, Candida albicans, or Yarrowia hpolytica.

In certain embodiments, the host cell is an algal cell such as, Chlamydomonas (e.g., C. Reinhardtii) and Phormidium (P. sp. ATCC29409).

In other embodiments, the host cell is a prokaryotic cell. Suitable prokaryotic cells include gram positive, gram negative, and gram-variable bacterial cells. The host cell may be a species of, but not limited to: Agrobacterium, Alicyclobacillus, Anabaena, Anacystis, Acinetobacter, Acidothermus, Arthrobacter, Azobacter, Bacillus, Bifidobacterium, Brevibacterium, Butyrivibrio, Buchnera, Campestris, Camplyobacter, Clostridium, Corynebacterium, Chromatium, Coprococcus, Escherichia, Enterococcus, Enterobacter, Envinia, Fusobacterium, Faecalibacterium, Francisella, Flavobacterium, Geobacillus, Haemophilus, Helicobacter, Klebsiella, Lactobacillus, Lactococcus, Ilyobacter, Micrococcus, Microbacterium, Mesorhizobium, Methylobacterium, Methylobacterium, Mycobacterium, Neisseria, Pantoea, Pseudomonas, Prochlorococcus, Rhodobacter, Rhodopseudomonas, Rhodopseudomonas, Roseburia, Rhodospirillum, Rhodococcus, Scenedesmus, Streptomyces, Streptococcus, Synecoccus, Saccharomonospora, Saccharopolyspora, Staphylococcus, Serratia, Salmonella, Shigella, Thermoanaerobacterium, Tropheryma, Tularensis, Temecula, Thermosynechococcus, Thermococcus, Ureaplasma, Xanthomonas, Xylella, Yersinia, and Zymomonas. In some embodiments, the host cell is Corynebacterium glutamicum.

In some embodiments, the bacterial host strain is an industrial strain. Numerous bacterial industrial strains are known and suitable in the methods and compositions described herein.

In some embodiments, the bacterial host cell is of the Agrobacterium species (e.g., A. radiobacter, A. rhizogenes, A. rubi), the Arthrobacter species (e.g., A. aurescens, A. citreus, A. globformis, A. hydrocarboglutamicus, A. mysorens, A. nicotianae, A. paraffineus, A. protophonniae, A. roseoparaffinus, A. sulfureus, A. ureafaciens), the Bacillus species (e.g., B. thuringiensis, B. anthracis, B. megaterium, B. subtilis, B. lentus, B. circulars, B. pumilus, B. lautus, B. coagulans, B. brevis, B. firmus, B. alkaophius, B. licheniformis, B. clausii, B. stearothermophilus, B. halodurans and B. amyloliquefaciens). In particular embodiments, the host cell will be an industrial Bacillus strain including but not limited to B. subtilis, B. pumilus, B. licheniformis, B. megaterium, B. clausii, B. stearothermophilus and B. amyloliquefaciens. In some embodiments, the host cell will be an industrial Clostridium species (e.g., C. acetobutylicum, C. tetani E88, C. lituseburense, C. saccharobutylicum, C. perfringens, C. beijerinckii). In some embodiments, the host cell will be an industrial Corynebacterium species (e.g., C. glutamicum, C. acetoacidophilum). In some embodiments, the host cell will be an industrial Erwinia species (e.g., E. uredovora, E. carotovora, E. ananas, E. herbicola, E. punctata, E. terreus). In some embodiments, the host cell will be an industrial Pantoea species (e.g., P. citrea, P. agglomerans). In some embodiments, the host cell will be an industrial Pseudomonas species, (e.g., P. putida, P. aeruginosa, P. mevalonii). In some embodiments, the host cell will be an industrial Streptococcus species (e.g., S. equisimiles, S. pyogenes, S. uberis). In some embodiments, the host cell will be an industrial Streptomyces species (e.g., S. ambofaciens, S. achromogenes, S. avermitilis, S. coelicolor, S. aureofaciens, S. aureus, S. fungicidicus, S. griseus, S. lividans). In some embodiments, the host cell will be an industrial Zymomonas species (e.g., Z. mobilis, Z. hpolytica), and the like.

In some embodiments, the host cell will be an industrial Escherichia species (e.g., E. coli).

Suitable host strains of the E. coli species comprise: Enterotoxigenic E. coli (ETEC), Enteropathogenic E. coli (EPEC), Enteroinvasive E. coli (EIEC), Enterohemorrhagic E. coli (EHEC), Uropathogenic E. coli (UPEC), Verotoxin-producing E. coli, E. coli O157:H7, E. coli O104:H4, Escherichia coli O121, Escherichia coli O104:H21, Escherichia coli K1, and Escherichia coli NC101. In some embodiments, the present disclosure teaches genomic engineering of E. coli K12, E. coli B, and E. coli C.

In some embodiments, the host cell can be E. coli strains NCTC 12757, NCTC 12779, NCTC 12790, NCTC 12796, NCTC 12811, ATCC 11229, ATCC 25922, ATCC 8739, DSM 30083, BC 5849, BC 8265, BC 8267, BC 8268, BC 8270, BC 8271, BC 8272, BC 8273, BC 8276, BC 8277, BC 8278, BC 8279, BC 8312, BC 8317, BC 8319, BC 8320, BC 8321, BC 8322, BC 8326, BC 8327, BC 8331, BC 8335, BC 8338, BC 8341, BC 8344, BC 8345, BC 8346, BC 8347, BC 8348, BC 8863, and BC 8864.

In some embodiments, the present disclosure teaches host cells that can be verocytotoxigenic E. coli (VTEC), such as strains BC 4734 (O26:H11), BC 4735 (O157:H-), BC 4736, BC 4737 (n.d.), BC 4738 (O157:H7), BC 4945 (O26:H-), BC 4946 (O157:H7), BC 4947 (O111:H-), BC 4948 (O157:H), BC 4949 (O5), BC 5579 (O157:H7), BC 5580 (O157:H7), BC 5582 (O3:H), BC 5643 (O2:H5), BC 5644 (O128), BC 5645 (O55:H-), BC 5646 (O69:H-), BC 5647 (O101:H9), BC 5648 (O103:H2), BC 5850 (O22:H8), BC 5851 (O55:H-), BC 5852 (O48:H21), BC 5853 (O26:H11), BC 5854 (O157:H7), BC 5855 (O157:H-), BC 5856 (O26:H-), BC 5857 (O103:H2), BC 5858 (O26:H11), BC 7832, BC 7833 (Oraw form:H-), BC 7834 (ONT:H-), BC 7835 (O103:H2), BC 7836 (O57:H-), BC 7837 (ONT:H-), BC 7838, BC 7839 (O128:H2), BC 7840 (O157:H-), BC 7841 (O23:H-), BC 7842 (O157:H-), BC 7843, BC 7844 (O157:H-), BC 7845 (O103:H2), BC 7846 (O26:H11), BC 7847 (O145:H-), BC 7848 (O157:H-), BC 7849 (O156:H47), BC 7850, BC 7851 (O157:H-), BC 7852 (O157:H-), BC 7853 (O5:H-), BC 7854 (O157:H7), BC 7855 (O157:H7), BC 7856 (O26:H-), BC 7857, BC 7858, BC 7859 (ONT:H-), BC 7860 (O129:H-), BC 7861, BC 7862 (O103:H2), BC 7863, BC 7864 (Oraw form:H-), BC 7865, BC 7866 (O26:H-), BC 7867 (Oraw form:H-), BC 7868, BC 7869 (ONT:H-), BC 7870 (O113:H-), BC 7871 (ONT:H-), BC 7872 (ONT:H-), BC 7873, BC 7874 (Oraw form:H-), BC 7875 (O157:H-), BC 7876 (O111:H-), BC 7877 (O146:H21), BC 7878 (O145:H-), BC 7879 (O22:H8), BC 7880 (Oraw form:H-), BC 7881 (O145:H-), BC 8275 (O157:H7), BC 8318 (O55:K-:H-), BC 8325 (O157:H7), and BC 8332 (ONT), BC 8333.

In some embodiments, the present disclosure teaches host cells that can be enteroinvasive E. coli (EIEC), such as strains BC 8246 (O152:K-:H-), BC 8247 (O124:K(72):H3), BC 8248 (O124), BC 8249 (O112), BC 8250 (O136:K(78):H-), BC 8251 (O124:H-), BC 8252 (O144:K-:H-), BC 8253 (O143:K:H-), BC 8254 (O143), BC 8255 (O112), BC 8256 (O28a.e), BC 8257 (O124:H-), BC 8258 (O143), BC 8259 (O167:K-:H5), BC 8260 (O128a.c.:H35), BC 8261 (O164), BC 8262 (O164:K-:H-), BC 8263 (O164), and BC 8264 (O124).

In some embodiments, the present disclosure teaches host cells that can be enterotoxigenic E. coli (ETEC), such as strains BC 5581 (O78:H11), BC 5583 (O2:K1), BC 8221 (O118), BC 8222 (O148:H-), BC 8223 (O111), BC 8224 (O110:H-), BC 8225 (O148), BC 8226 (O118), BC 8227 (O25:H42), BC 8229 (O6), BC 8231 (O153:H45), BC 8232 (O9), BC 8233 (O148), BC 8234 (O128), BC 8235 (O118), BC 8237 (O111), BC 8238 (O110:H17), BC 8240 (O148), BC 8241 (O6H16), BC 8243 (O153), BC 8244 (O15:H-), BC 8245 (O20), BC 8269 (O125a.c:H-), BC 8313 (O6:H6), BC 8315 (O153:H-), BC 8329, BC 8334 (O118:H12), and BC 8339.

In some embodiments, the present disclosure teaches host cells that can be enteropathogenic E. coli (EPEC), such as strains BC 7567 (O86), BC 7568 (O128), BC 7571 (O114), BC 7572 (O119), BC 7573 (O125), BC 7574 (O124), BC 7576 (O127a), BC 7577 (O126), BC 7578 (O142), BC 7579 (O26), BC 7580 (OK26), BC 7581 (O142), BC 7582 (O55), BC 7583 (O158), BC 7584 (O-), BC 7585 (O-), BC 7586 (O-), BC 8330, BC 8550 (O26), BC 8551 (O55), BC 8552 (O158), BC 8553 (O26), BC 8554 (O158), BC 8555 (O86), BC 8556 (O128), BC 8557 (OK26), BC 8558 (O55), BC 8560 (O158), BC 8561 (O158), BC 8562 (O114), BC 8563 (O86), BC 8564 (O128), BC 8565 (O158), BC 8566 (O158), BC 8567 (O158), BC 8568 (O111), BC 8569 (O128), BC 8570 (O114), BC 8571 (O128), BC 8572 (O128), BC 8573 (O158), BC 8574 (O158), BC 8575 (O158), BC 8576 (O158), BC 8577 (O158), BC 8578 (O158), BC 8581 (O158), BC 8583 (O128), BC 8584 (O158), BC 8585 (O128), BC 8586 (O158), BC 8588 (O26), BC 8589 (O86), BC 8590 (O127), BC 8591 (O128), BC 8592 (O114), BC 8593 (O114), BC 8594 (O114), BC 8595 (O125), BC 8596 (O158), BC 8597 (O26), BC 8598 (O26), BC 8599 (O158), BC 8605 (O158), BC 8606 (O158), BC 8607 (O158), BC 8608 (O128), BC 8609 (O55), BC 8610 (O114), BC 8615 (O158), BC 8616 (O128), BC 8617 (O26), BC 8618 (O86), BC 8619, BC 8620, BC 8621, BC 8622, BC 8623, BC 8624 (O158), and BC 8625 (O158).

In some embodiments, the present disclosure also teaches host cells that can be Shigella organisms, including Shigelia flexneri, Shigella dysenteriae, Shigella boydii, and Shigella sonnei.

The present disclosure is also suitable for use with a variety of animal cell types, including mammalian cells, for example, human (including 293, WI38, PER.C6 and Bowes melanoma cells), mouse (including 3T3, NS0, NS1, Sp2/0), hamster (CHO, BHK), monkey (COS, FRhL, Vero), and hybridoma cell lines.

In various embodiments, strains that may be used in the practice of the disclosure including both prokaryotic and eukaryotic strains, are readily accessible to the public from a number of culture collections such as American Type Culture Collection (ATCC), Deutsche Sammlung von Mikroorganismen and Zellkulturen GmbH (DSM), Centraalbureau Voor Schimmelcultures (CBS), and Agricultural Research Service Patent Culture Collection, Northern Regional Research Center (NRRL).

In some embodiments, the methods of the present disclosure are also applicable to multi-cellular organisms. For example, the platform could be used for improving the performance of crops. The organisms can comprise a plurality of plants such as Gramineae, Fetucoideae, Poacoideae, Agrostis, Phleum, Dactylis, Sorgum, Setaria, Zea, Oryza, Triticum, Secale, Avena, Hordeum, Saccharum, Poa, Festuca, Stenotaphrum, Cynodon, Coix, Olyreae, Phareae, Compositae or Leguminosae. For example, the plants can be corn, rice, soybean, cotton, wheat, rye, oats, barley, pea, beans, lentil, peanut, yam bean, cowpeas, velvet beans, clover, alfalfa, lupine, vetch, lotus, sweet clover, wisteria, sweet pea, sorghum, millet, sunflower, canola or the like. Similarly, the organisms can include a plurality of animals such as non-human mammals, fish, insects, or the like.

### Sequencing Methods

In one embodiment, the molecular analysis steps of the enrichment methods provided herein utilize first generation sequencing methods or platforms. An example of a first generation sequencing method for use in the enrichment methods provided herein can be classic dideoxy sequencing reactions (Sanger method) using labeled terminators or primers and gel separation in slab or capillary.

In one embodiment, the molecular analysis steps of the enrichment methods provided herein utilize next generation sequencing (NGS) methods or platforms. The enrichment methods provided herein (e.g., CS-seq and SG-seq) can produce amplicons that are sequenced using the method commercialized by Illumina, as described U.S. Pat. Nos. 5,750,341; 6,306,597; and 5,969,119.

In some embodiments, the enrichment methods provided herein (e.g., CS-seq and SG-seq) are useful for preparing amplicons for sequencing by the sequencing by ligation methods commercialized by Applied Biosystems (e.g., SOLiD sequencing). In other embodiments, the methods are useful for preparing amplicons for sequencing by synthesis using the methods commercialized by 454/Roche Life Sciences, including but not limited to the methods and apparatus described in Margulies et al., Nature (2005) 437:376-380 (2005); and U.S. Pat. Nos. 7,244,559; 7,335,762; 7,211,390; 7,244,567; 7,264,929; and 7,323,305. In other embodiments, the methods are useful for preparing amplicons for sequencing by the methods commercialized by Helicos BioSciences Corporation (Cambridge, Mass.) as described in U.S. application Ser. No. 11/167,046, and U.S. Pat. Nos. 7,501,245; 7,491,498; 7,276,720; and in U.S. Patent Application Publication Nos. US20090061439; US20080087826; US20060286566; US20060024711; US20060024678; US20080213770; and US20080103058. In other embodiments, the methods are useful for preparing amplicons for sequencing by the methods commercialized by Pacific Biosciences as described in U.S. Pat. Nos. 7,462,452; 7,476,504; 7,405,281; 7,170,050; 7,462,468; 7,476,503; 7,315,019; 7,302,146; 7,313,308; and US Application Publication Nos. US20090029385; US20090068655; US20090024331; and US20080206764.

Another example of a sequencing technique that can be used in the enrichment methods provided herein is nanopore sequencing (see e.g. Soni G V and Meller A. (2007) Clin Chem 53: 1996-2001). A nanopore can be a small hole of the order of 1 nanometer in diameter. Immersion of a nanopore in a conducting fluid and application of a potential across it can result in a slight electrical current due to conduction of ions through the nanopore. The amount of current that flows is sensitive to the size of the nanopore. As a DNA molecule passes through a nanopore, each nucleotide on the DNA molecule obstructs the nanopore to a different degree. Thus, the change in the current passing through the nanopore as the DNA molecule passes through the nanopore can represent a reading of the DNA sequence.

Another example of a sequencing technique that can be used in the enrichment methods provided herein is semiconductor sequencing provided by Ion Torrent (e.g., using the Ion Personal Genome Machine (PGM)). Ion Torrent technology can use a semiconductor chip with multiple layers, e.g., a layer with micro-machined wells, an ion-sensitive layer, and an ion sensor layer. Nucleic acids can be introduced into the wells, e.g., a clonal population of single nucleic can be attached to a single bead, and the bead can be introduced into a well. To initiate sequencing of the nucleic acids on the beads, one type of deoxyribonucleotide (e.g., dATP, dCTP, dGTP, or dTTP) can be introduced into the wells. When one or more nucleotides are incorporated by DNA polymerase, protons (hydrogen ions) are released in the well, which can be detected by the ion sensor. The semiconductor chip can then be washed and the process can be repeated with a different deoxyribonucleotide. A plurality of nucleic acids can be sequenced in the wells of a semiconductor chip. The semiconductor chip can comprise chemical-sensitive field effect transistor (chemFET) arrays to sequence DNA (for example, as described in U.S. Patent Application Publication No. 20090026082). Incorporation of one or more triphosphates into a new nucleic acid strand at the 3′ end of the sequencing primer can be detected by a change in current by a chemFET. An array can have multiple chemFET sensors.

In one aspect of the disclosure, high-throughput methods of NGS are employed that comprise a step of spatially isolating individual molecules on a solid surface where they are sequenced in parallel. Such solid surfaces may include nonporous surfaces (such as in Solexa sequencing, e.g. Bentley et al, Nature, 456: 53-59 (2008) or Complete Genomics sequencing, e.g. Drmanac et al, Science, 327: 78-81 (2010)), arrays of wells, which may include bead- or particle-bound templates (such as with 454, e.g. Margulies et al, Nature, 437: 376-380 (2005) or Ion Torrent sequencing, U.S. patent publication 2010/0137143 or 2010/0304982), micromachined membranes (such as with SMRT sequencing, e.g. Eid et al, Science, 323: 133-138 (2009)), or bead arrays (as with SOLiD sequencing or polony sequencing, e.g. Kim et al, Science, 316: 1481-1414 (2007)).

In another embodiment, the methods of the present disclosure comprise amplifying the isolated molecules either before or after they are spatially isolated on a solid surface. Prior amplification may comprise emulsion-based amplification, such as emulsion PCR, or rolling circle amplification. Also taught is Solexa-based sequencing where individual template molecules are spatially isolated on a solid surface, after which they are amplified in parallel by bridge PCR to form separate clonal populations, or clusters, and then sequenced, as described in Bentley et al (cited above) and in manufacturer's instructions (e.g. TruSeq™ Sample Preparation Kit and Data Sheet, Illumina, Inc., San Diego, Calif., 2010); and further in the following references: U.S. Pat. Nos. 6,090,592; 6,300,070; 7,115,400; and EP0972081B1; which are incorporated by reference.

In one embodiment, the molecular analysis steps of the enrichment methods provided herein utilize third generation sequencing methods or platforms. Further to this embodiment, when employing third generation sequencing (e.g., Oxford Nanopore Technologies MinION sequencing) during the molecular analysis step, one or more adapter sequence(s) are appended to the amplicons produced in CS-seq and used to perform said third generation sequencing (e.g., Nanopore adapter sequence). Further to the above embodiment, when employing third generation sequencing (e.g., Oxford Nanopore Technologies MinION sequencing) during the molecular analysis step, one or more adapter sequence(s) are appended to the amplicons produced in SG-seq and used to perform said third generation sequencing (e.g., Nanopore adapter sequence). An example of third generation sequencing methods for use in the enrichment methods provided herein can be Pacific Biosciences (PacBio) Single Molecule Real Time (SMRT) sequencing, the Illumina Tru-seq Synthetic Long-Read technology and the Oxford Nanopore Technologies MinION Technologies sequencing platform. Using single-molecule sequencing or clonal amplification and sequencing of long molecules, all three technologies can produce long reads averaging between 5,000 bp to 15,000 bp, with some reads exceeding 100,000 bp.

### Alignment Methods

As provided herein, the molecular analysis portion of the enrichment methods provided herein (e.g., CS-seq and SG-seq) can comprise comparing sequence reads obtained from sequencing of the amplicons to a reference database for the organism (e.g., microbe) subjected to genetic engineering and subsequent targeted enrichment analysis using a computer-implemented method. The computer-implemented method can utilize a sequence similarity search program, a sequence composition search program or a combination thereof. In one embodiment, the sequence comparison is performed using any sequence similarity search program, sequence composition search program for performing global or local sequence alignment known in the art such as, for example, the programs discussed in Bazinet et al., BMC Bioinformatics 2012, 13:92. In some aspects, the alignment is accomplished by employing a program that utilizes the Smith-Waterman algorithm or the Needleman-Wunsch algorithm. In one embodiment, the sequence similarity search program employs a basic local alignment search tool (BLAST), fuzzy logic, lowest common ancestor (LCA) algorithm or a profile hidden Markov Model (pHMM). Examples of sequence similarity based alignment programs for use in the methods provided herein can include a BLAST algorithm, Bowtie, vsearch, usearch, NW-align, GGSEARCH, GLSEARCH, DNASTAR, JAligner, DNADot, ALLALIGN, ACANA, needle, matcher, NW, water, CARMA, FACS, jMOTU/Taxonerator, MARTA, MEGAN, MetaPhyler, MG-RAST, MTR, and SOrt-ITEMS and wordmatch. The sequence composition search program can employ interpolated Markov models (IMMs), naive Bayesian classifiers, k-mers or k-means/k-nearest-neighbor algorithms. Examples of sequence composition search programs for use in the methods provided herein can include Naive Bayes Classifier (NBC), PhyloPythia, PhymmBL, RAlphy, RDP, Scimm and TACOA The sequence comparison can be also be performed using computer implemented methods that employ programs that use a combination of a sequence similarity search and sequence composition search program such as, for example, fuzzy logic analysis of k-mers (FLAK) and SPHINX.

In one embodiment, the sequence composition search program employs k-mers. The k-mers can comprise short nucleotide sequences comprising nucleotide bases complementary to a sequence near the one or each of the plurality of genetic edits. In one embodiment, detection of the short nucleotide sequence in the sequence reads indicates presence of the one or each of the plurality of genetic edits in the microbial strain. The sequence near the one or each of the plurality of genetic edits can be as long as a sequencing read length, including but not limited to 300 base pairs (bps), 250 bps, 150 bps, 100 bps, 95 bps, 90 bps, 85 bps, 80 bps, 75 bps, 70 bps, 65 bps, 60 bps, 55 bps, 50 bps, 45 bps, 40 bps, 35 bps, 30 bps, 25 bps, 20 bps, 15 bps, 10 bps, or 5 bps of the one or each of the plurality of genetic edits. The sequence near the one or each of the plurality of genetic edits can be about 100 bps, about 95 bps, about 90 bps, about 85 bps, about 80 bps, about 75 bps, about 70 bps, about 65 bps, about 60 bps, about 55 bps, about 50 bps, about 45 bps, about 40 bps, about 35 bps, about 30 bps, about 25 bps, about 20 bps, about 15 bps, about 10 bps, or about 5 bps of the one or each of the plurality of genetic edits. The sequence near the one or each of the plurality of genetic edits can be within at least 100 bps, 95 bps, 90 bps, 85 bps, 80 bps, 75 bps, 70 bps, 65 bps, 60 bps, 55 bps, 50 bps, 45 bps, 40 bps, 35 bps, 30 bps, 25 bps, 20 bps, 15 bps, 10 bps, or 5 bps of the one or each of the plurality of genetic edits. The sequence near the one or each of the plurality of genetic edits can be within at most 100 bps, 95 bps, 90 bps, 85 bps, 80 bps, 75 bps, 70 bps, 65 bps, 60 bps, 55 bps, 50 bps, 45 bps, 40 bps, 35 bps, 30 bps, 25 bps, 20 bps, 15 bps, 10 bps, or 5 bps of the one or each of the plurality of genetic edits. The sequence near the one or each of the plurality of genetic edits can be between 100 bps-95 bps, between 95 bps-90 bps, between 90 bps-85 bps, between 85 bps-80 bps, between 80 bps-75 bps, between 75 bps-70 bps, between 70 bps-65 bps, between 65 bps-60 bps, between 60 bps-55 bps, between 55 bps-50 bps, between 50 bps-45 bps, between 45 bps-40 bps, between 40 bps-35 bps, between 35 bps-30 bps, between 30 bps-25 bps, between 25 bps-20 bps, between 20 bps-15 bps, between 15 bps-10 bps, or between 10 bps-5 bps of the one or each of the plurality of genetic edits. In one embodiment, the sequence near the one or each of the plurality of genetic edits is within 25 base pairs (bps), 20 bps, 15 bps, 10 bps, or 5 bps of the one or each of the plurality of genetic edits

### Automation

In one embodiment, the kits, compositions and methods provided herein are incorporated into a high-throughput (HTP) method for genetic engineering and screening of an organism (e.g., a microbial host cell). In another embodiment, the methods provided herein can be implemented as an additional tool to be used in combination or conjunction with the one or more molecular tools that are part of the suite of HTP molecular tool sets described in WO 2018/226900, WO 2018/226880 or WO 2017/100377, each of which is herein incorporated by reference, for all purposes, to create and screen genetically engineered microbial host cells with a desired trait or phenotype. Examples of libraries that can be generated using the methods provided herein to iteratively edit the genome of a microbial host cell can include, but are not limited to promoter ladders, terminator ladders, solubility tag ladders or degradation tag ladders. Examples of high-throughput genomic engineering methods for which the methods provided herein can be used to genotype and identify the presence and/or location of one or more genetic edits in resultant strains generated by said high-throughput genomic engineering methods can include, but are not limited to, promoter swapping, terminator (stop) swapping, solubility tag swapping, degradation tag swapping or SNP swapping as described in WO 2018/226900, WO 2018/226880 or WO 2017/100377. Like the high-throughput genomic engineering methods described above, the enrichment methods provided herein (e.g., CS-seq and SG-seq) can be automated and/or utilize robotics and liquid handling platforms (e.g., plate robotics platform and liquid handling machines known in the art. The high-throughput methods can utilize multi-well plates such as, for example microtiter plates.

In some embodiments, the automated methods of the disclosure comprise a robotic system. The systems outlined herein are generally directed to the use of 96- or 384-well microtiter plates, but as will be appreciated by those in the art, any number of different plates or configurations may be used. In addition, any or all of the steps outlined herein may be automated; thus, for example, the systems may be completely or partially automated. The robotic systems compatible with the methods and compositions provided herein can be those described in WO 2018/226900, WO 2018/226880 or WO 2017/100377.

### Kits

Any of the compositions described herein may be comprised in a kit. In a non-limiting example, the kit, in a suitable container, comprises: an adaptor or several adaptors, one or more of oligonucleotide primers and reagents for ligation, primer extension and amplification. The kit may also comprise means for purification, such as a bead suspension.

The containers of the kits will generally include at least one vial, test tube, flask, bottle, syringe or other containers, into which a component may be placed, and preferably, suitably aliquoted. Where there is more than one component in the kit, the kit also will generally contain a second, third or other additional container into which the additional components may be separately placed. However, various combinations of components may be comprised in a container.

When the components of the kit are provided in one or more liquid solutions, the liquid solution can be an aqueous solution. However, the components of the kit may be provided as dried powder(s). When reagents and/or components are provided as a dry powder, the powder can be reconstituted by the addition of a suitable solvent.

A kit will preferably include instructions for employing, the kit components as well the use of any other reagent not included in the kit. Instructions may include variations that can be implemented.

In one aspect, the invention provides kits containing any one or more of the elements disclosed in the above methods and compositions. In some embodiments, a kit comprises a composition provided herein for use in performing CS-seq or SG-seq as provided herein, in one or more containers. In some embodiments, kits for performing CS-seq comprise adapters, primers, and/or reagents for performing tagmentation, PCR, size selection and/or sequencing as described herein. In some embodiments, kits for performing SG-seq comprise primers including semi-guided primers as provided herein and/or reagents for performing PCR, size selection and/or sequencing as described herein. The kits provided herein may further comprise additional agents, such as those described above, for use according to the methods of the invention. The kit elements can be provided in any suitable container, including but not limited to test tubes, vials, flasks, bottles, ampules, syringes, or the like. The agents can be provided in a form that may be directly used in the methods of the invention, or in a form that requires preparation prior to use, such as in the reconstitution of lyophilized agents. Agents may be provided in aliquots for single-use or as stocks from which multiple uses, such as in a number of reaction, may be obtained.

### EXAMPLES

The present invention is further illustrated by reference to the following Examples. However, it should be noted that these Examples, like the embodiments described above, are illustrative and are not to be construed as restricting the scope of the invention in any way.

### Example 1—Proof of Principle for CS-Seq Enrichment Method for Detecting Genetic Edits Using Tagmentation

### Objective

This example describes the use of a CS-Seq enrichment method employing tagmentation to identify genetic edits introduced into the genome of a microbial host cell.

### Materials and Methods

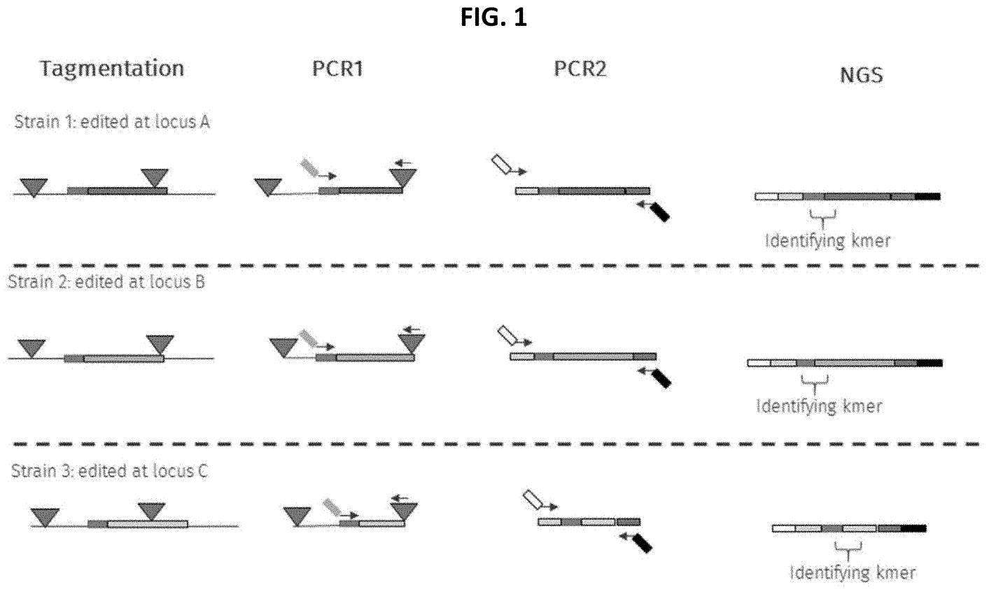

Genomic DNA was extracted from 3 separate E. coli strains each containing a single edit at one of 3 possible loci within the E. coli genome (i.e., locus A, locus B and locus C). The same genetic edit (i.e., an exogenous promoter sequence) was targeted for insertion at one of the targeted loci (i.e., locus A, locus B and locus C). Following genomic DNA extraction, libraries for subsequent next-generation sequencing (NGS) were generated from said genomic DNA by subjecting said genomic DNA to Nextera® Tagmentation in order to fragment the genomic DNA and append adapters to said genomic DNA fragments. The adapters added during tagmentation all contained a single universal sequence common to each adapter. Following tagmentation, the DNA fragments were subjected to the CS-seq enrichment method shown in FIG. 1 prior to molecular analysis by NGS.

As shown in FIG. 1, a first PCR (i.e., PCR1 in FIG. 1) was performed using a forward primer specific to the genetic edit inserted at each of the three loci (i.e., A, B and C) in the separate strains and a reverse primer specific to the universal sequence present in the adapter added to each DNA fragment during tagmentation of the genomic DNA extracted from each of the 3 strains. Table 1 shows the primer sequences used in the CS-seq method described in this example. For the first PCR, the PCR1-Fs primer comprised sequence that bound to a portion of the inserted genetic edit sequence (italicized portion of the PCR1-Fs primer in Table 1) at the 3′ end of the primer and TruSeq adapter sequence in a non-complementary portion of the primer found at the 5′ end. The PCR1-R primers used in the first PCR comprised sequence that was complementary to and bound to the adapter sequence added by Nextera Tagmentation reagent (grayed out part of PCR1-R in Table 1). The purpose of this step was to enrich for the portions of the genome of each of the 3 strains where the genetic edit inserted by specifically amplifying the genomic region of interest using primers that bound the integrated genetic edit and the nearest universal sequence present in the adapters added to the fragmented genomic DNA during tagmentation.

TABLE 1
CS-seq primer sequences.

| | |
|---|---|
| PCR1-Fs | GATCTACACTCTTTCCCTACACGACGCTCTTCCGATCTGCT |
| | AGCACTGTACCTAGGACTGAGCTAG (SEQ ID NO: 1) |
| PCR2-F | AATGATACGGCGACCACCGAGATCTACACCCATGTTGGCTCA |
| | TTGGAAACCACTACAGATCTACACTCTTTCCCTACACGACG |
| | CTCTTCCGATCT (SEQ ID NO: 2) |
| PCR1-R | 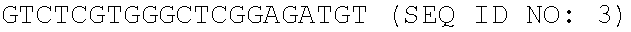 |
| PCR2-R | CAAGCAGAAGACGGCATACGAGATGCTGTGTTGATCATAGGCT |
| | 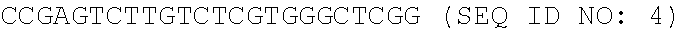 |

Subsequently, a second PCR step (i.e., PCR2 in FIG. 1) was performed on an aliquot of the amplicons produced during the first PCR on each of the 3 strains. The second PCR step used a forward primer (i.e., PCR2-F in Table 1) that comprised sequence complementary to the PCR1 forward primer TruSeq adapter sequence from the PCR1-Fs primer (bold portion of the PCR2-F primer in Table 1) and a reverse primer (i.e., PCR2-R in Table 1) that comprised sequence complementary to the tagmentation adapter from the PCR1-R primer, but offset by 6 nucleotides (grayed out portion of PCR2-R in Table 1). The PCR2 forward and reverse primers further comprised P5 and P7 Illumina adapter sequences, respectively, and an 8 base index sequence to allow sample identification after sequencing. As such, the PCR2-F primers bound to the TruSeq adapter added by the PCR1-F primer, while the PCR2-R primers bound to the adapter sequence added by Nextera Tagmentation reagent, but offset by 6 nucleotides. The P5 Illumina adapter sequence is the underlined portion of PCR2-F in Table 1, while the P7 Illumina adapter sequence is the italicized, underlined portion of PCR2-R in Table 1. The index sequence is the bold, underlined sequences in the PCR2-F and -R primers in Table 1. The purpose of this step was to use a common set of indexed primers to add unique sample indices to each sample and to also add the sequences required for sequencing on the Illumina MiSeq NGS platform (i.e. i5 and i7 sequences).

Subsequently, size selection and amplicon purification were performed using AmpureXP SPRI beads according to manufacturer's protocol (i.e., Beckman Coulter) to select for amplicons in the 200-400 bp range for use in the Illumina MiSeq based sequencing platform. Once obtained, chosen amplicons were subjected to NGS on the Illumina MiSeq platform. Raw sequences reads were aligned to potential edited sequence to determine which, if any, of the genetics edits were present in the genome of the respective strain at the desired loci (i.e., locus A, B or C). Strains yielding amplicons with NGS reads that aligned to the sequence of interest could then be tested for phenotype of genotype.

It should be noted that, as shown in FIG. 1, the NGS sequencing results could have also been analyzed by searching said sequence reads using short nucleotide sequences (i.e., k-mers) that were specific for a junction of interest. In this example, the k-mer for each of the 3 loci would be about 5-20 bases on either side of the junction between the inserted genetic edit and the locus (i.e., A, B or C) in the genome of the respective microbial strain.

### Results

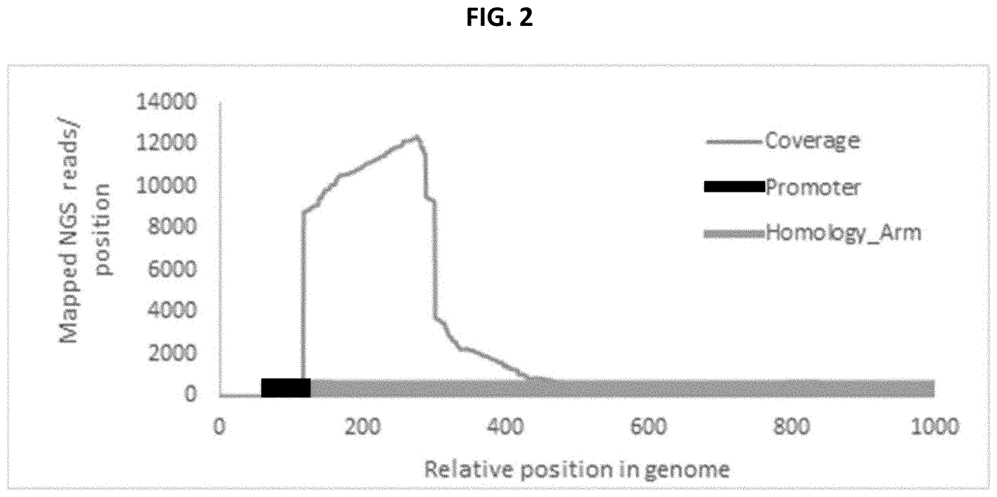

As shown in FIG. 2, while the entire genome of each strain was tagmented, only the junction between a sequence of interest (i.e., genetic edit or inserted promoter in this example) and the tagmentation site was amplified. Accordingly, the CS-seq method described in this example was effective in enriching the sequence reads obtained from the genomic DNA isolated from each the edited microbial strains for the junction between the inserted genetic edit (i.e., promoter sequence in FIG. 2) and the target insertion locus (i.e., homology arm portion of the genomic DNA sequence in FIG. 2). This approach can be used to identify a particular locus of insertion of one or more sequences of interest (e.g. Promoter in FIG. 2) when the strains are generated in a pooled fashion.

### Example 2—Proof of Principle for Detecting Genetic Edits Using SG-Seq

### Objective

This example describes the use and optimization of semi-guided sequencing (SG-Seq) enrichment methods to identify genetic edits introduced into the genome of a microbial host cell.

### Materials and Methods

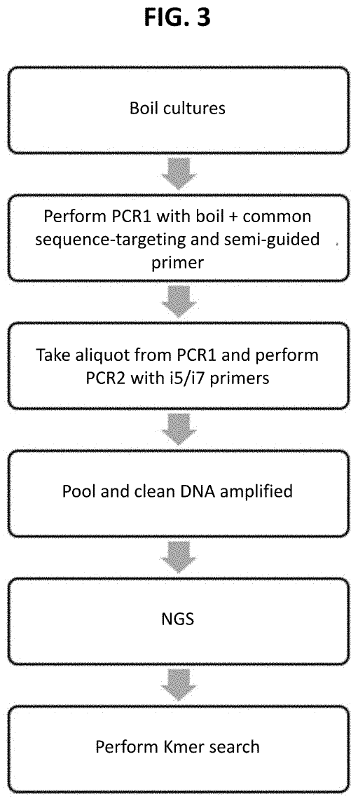

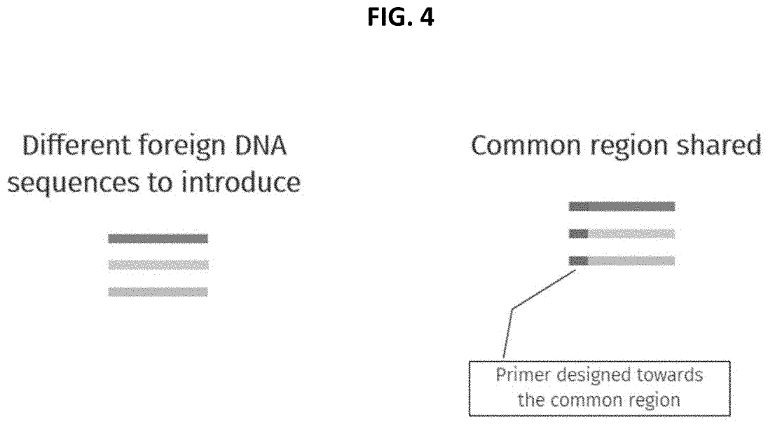

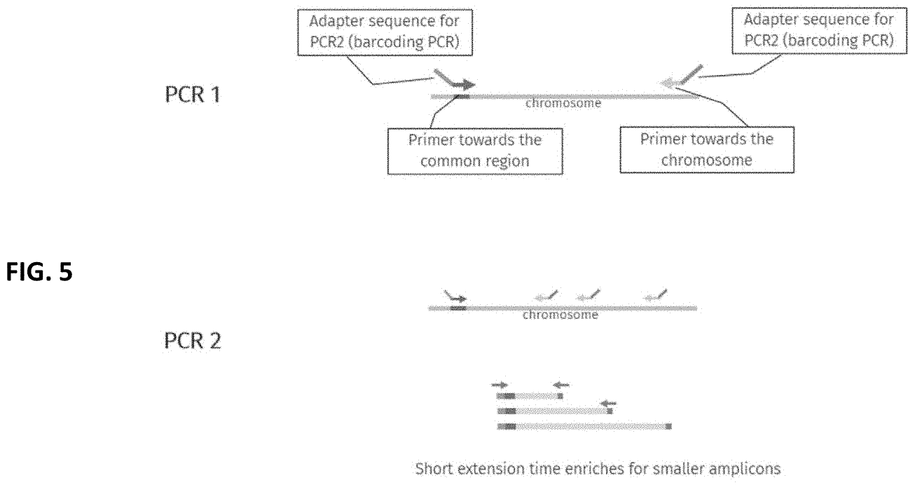

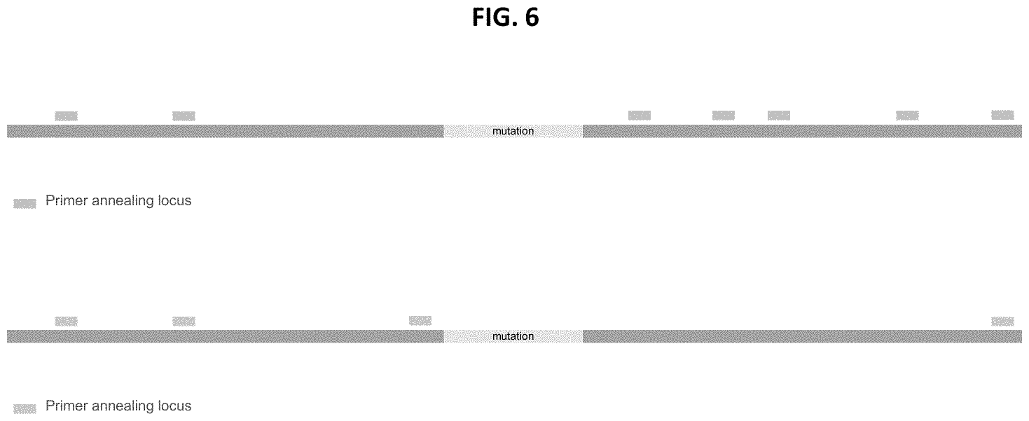

As outlined in FIG. 3, the embodiment of the SG-seq method described in this example encompassed performing two independent but linked rounds of PCR (i.e., PCR1 and PCR2 in FIG. 5) on boil preparations or genomic DNA extracted from cultures of edited microbial strains. In the first round (i.e., PCR1), the sequence of the inserted genetic edits were used to design a forward PCR primer comprising sequence complementary to a common sequence present in each genetic edit (see FIG. 4) and a 5′ overhang encoding non-complementary universal sequence. The reverse primer used in PCR1 was “semi-guided” and comprised 3-5 bases of defined sequence and multiple non-specified (degenerate or arbitrary) bases at its 3′ end and a specific overhang at the 5′ required for the second round of PCR (i.e., PCR2). The 3-5 defined bases (the “semi-guided” part) were found with a frequency that was enough to have at least one binding site near the locus of the genome where the genetic edit inserted, but still rare enough to prevent the primers from binding randomly at every spot in the genome of the edited cell or strain (see FIG. 6).

PCR1 was followed by PCR2 with an aliquot from PCR1 serving as template for PCR2, and employing a second set of primers. One primer in the second set was specific to the non-complementary universal sequence from the forward PCR primer used in PCR1 (and covered by the first round of PCR), while the other primer was specific to the overhang of the semi-guided primer of the first round of PCR (i.e., the reverse primer from PCR1). These specific primers also comprised 5′ overhangs that constituted the indices that specify the sample well identity similarly to the CS-Seq method provided throughout and described in Example 1. Table 2 provides details of the forward and reverse primers used in PCR1 (i.e., PCR1-F and PCR1-R primers) and PCR2 (i.e., PCR2-F and PCR2-R primers).

Size selection and purification were performed using AmpureXP SPRI beads as described in Example 1.

Upon pooling and cleanup of the size selected products from PCR2, sequencing libraries were prepared for 96 standard samples, and sequenced using Illumina MiSeq. The edits were identified using k-mer analysis as described for the CS-Seq method described in Example 1.

SG-seq PCR template prep:

To optimize the SG-seq method towards a low-cost, fast enrichment method, different variables with regards to the template DNA were tested. For example, (1) aliquots of different volumes from overnight cultures were used as well as (2) different volumes of different ratios of culture:water were boiled for 10 min to lyse the cells. The results shown in Table 3 reflect the results from using different volumes of different ratios of culture:water (i.e., (2)).

SG-seq Enrichment (PCR1) and indexing (PCR2) optimization:

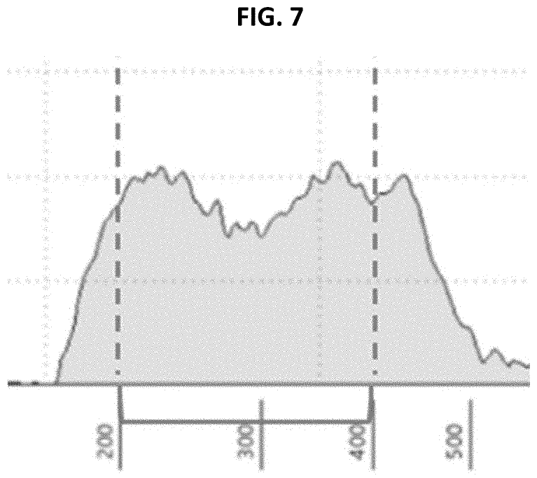

In addition to the optimization of the template source, the (1) number of cycles of PCR1, (2) semi-guided primer for PCR1 (PCR1-R primers in Table 2) and the (3) extension times for PCR2 were also varied. By varying (1), the goal was to enrich the PCR only for the target sequences, by varying (2) and (3) the goal was to increase the number of annealing loci (see FIG. 6) and decrease amplicon length (i.e., aiming to obtain the desired amplicon size (˜300 bp) for NGS (see FIG. 7)), respectively.

TABLE 2
SG-seq primer sequences.

| | |
|---|---|
| SG-seq | TCGTCGGCAGCGTCTATTTACCTCCTTTATGCTAGCA |
| PCR1-F | (SEQ ID NO: 5) |
| SG-seq | AATGATACGGCGACCACCGAGATCTACACCCATGTTGGCTCA |
| PCR2-F | TTGGAAACCACTACATCGTCGGCAGCGTC |
| | (SEQ ID_NO: 6) |
| SG-seq | GTCTCGTGGGCTCGGNNNNNNNNNNTGCGG |
| PCR1-R | (SEQ ID_NO: 7) |
| | or |
| | GTCTCGTGGGCTCGGNNNNNNNNNNNGCGG |
| | (SEQ ID_NO: 8) |
| | or |
| | GTCTCGTGGGCTCGGNNNNNNNNNNNNCGG |
| | (SEQ ID_NO: 9) |
| | or |
| | GTCTCGTGGGCTCGGNNNNNNNNNNCTATA |
| | (SEQ ID_NO: 10) |
| | or |
| | GTCTCGTGGGCTCGGNNNNNNNNNNNTATA |
| | (SEQ ID_NO: 11) |
| | or |
| | GTCTCGTGGGCTCGGNNNNNNNNNNNNATA |
| | (SEQ ID_NO: 12) |
| SG-seq | CAAGCAGAAGACGGCATACGAGATGCTGTGTTGATCATAGG |
| PCR2-R | CTCCGAGTCTTGTCTCGTGGGCTCGG |
| | (SEQ ID_NO: 13) |

*For PCR1-F, the bold is adapter sequence for indexing primer region (PCR2-F), while the underlined is sequence directed towards the genetic edit being introduced and to be searched for.
For PCR1-R, the italicized is adapter sequence for indexing primer region (PCR2-R), while the remaining sequence is the semi-guided portion of the primer. The index sequence is the bold, underlined sequences in the PCR2-F and -R primers.

### Results

Upon PCR protocol and primer (Table 2, PCR1-R) optimization, it was possible to generate SG libraries averaging 300 bp in size—ideal for NGS with Illumina MiSeq platform (see FIG. 7). The ultimate results of the PCR protocol and primer optimization experiments indicated that PCR1-R with SEQ ID NO: 7 was the best PCR1-R of those tested. Accordingly, the results shown in Table 3 reflect the results from using different volumes of different ratios of culture:water (i.e., (2)) as indicated in Table 3 using the PCR1-R with SEQ ID NO: 7 as compared with those expected as shown in Table 3. As can be seen in Table 3, SG-seq was capable of picking up all edits, while returning no false positives (did not pick up edits that did not exist). In particular, treatment T2 and T3 yielded 100% successful hit identification, with no false positive calls.

TABLE 3
Comparison of the edit detection and false positive (different
call) rate from SG-Seq analyses vs expected

| Treatments | | Correct call | Different call | No read |
|---|---|---|---|---|
| T1 | 5 culture + 15 H2O | 54 | 1 | 7 |
| | BP→ 2 uL used as | | | |
| | template | | | |
| T2 | 5 culture + 15 H2O | 62 | 0 | 0 |
| | BP→ 6 uL used as | | | |
| | template | | | |
| T3 | 10 culture + 10 H2O | 62 | 0 | 0 |
| | BP→ 2 uL used as | | | |
| | template | | | |
| T4 | 10 culture + 10 H2O | 58 | 0 | 4 |
| | BP→ 6 uL used as | | | |
| | template | | | |

### Example 3—Proof of Principle for Use of CS-Seq Enrichment Method for Detecting Ectopic Integration

### Objective

This example describes the use of the CS-Seq enrichment method to identify ectopic integration of genetic edits introduced into the genome of a S. cerevisiae host cell.

### Materials and Methods/Results

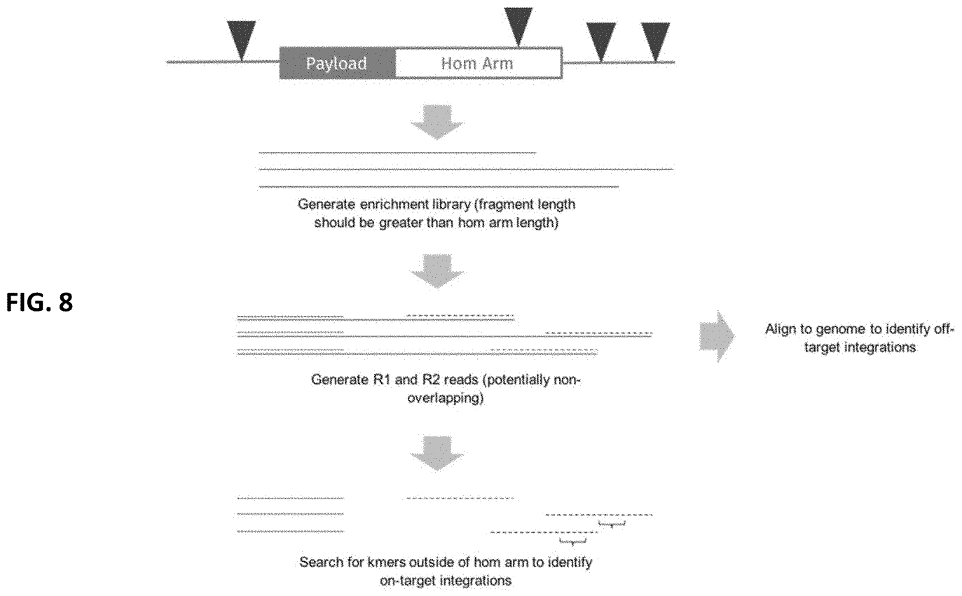

The general strategy to identify ectopic integrations is shown in FIG. 8. The key variables in obtaining the 300-700 bp library fragments used in this experiment were (1) the ratio of gDNA to tagmentation reagent, (2) the number of cycles used in the enrichment and indexing PCR reactions, and (3) the polymerase.

PCR template prep: Genomic DNA was extracted from liquid cultures of Saccharomyces cerevisiae originating from single colonies using a MagBio gDNA extraction kit. Concentrations were determined using a pico green assay. Since the ratio of transposase to gDNA would affect the library size, the amount of gDNA used for tagmentation was varied between 167 pg and 2 ng. Using 1-2 ng of yeast gDNA in a 417 nL reaction, combined with the PCR optimizations described below, gave libraries of 300-700 bps.

Enrichment (PCR1) and indexing (PCR2) optimization: The number of cycles and polymerase used were varied in order to obtain larger average fragment lengths. It was suspected that shorter fragments would be preferentially amplified, creating a size bias with increasing number of PCR cycles, so 14 and 20 cycles were tested. The yeast genome is AT-rich and it was suspected that certain polymerases may be better suited than others to amplify those sequences. OneTaq and Q5 polymerases were tested in initial experiments. The combination of Q5 polymerase and 14 cycles of amplification gave good yields with the longest library lengths (i.e., 300-700 bps); these conditions were used for both enrichment and amplification PCR.

Aside from these optimizations, the sequencing libraries were prepared as described above in Examples 1 for CS-seq. In PCR1, insert or common-specific (i.e., payload) forward primers and a constant reverse primer were used as described in FIG. 8. A common set of index primers were used in PCR2. After the second amplification, libraries were pooled, concentrated, and purified using a Zymo DNA clean and concentrate kit. Libraries were sequenced on an Illumina MiSeq (2×150 bp reads) by a third party vendor using standard procedures.

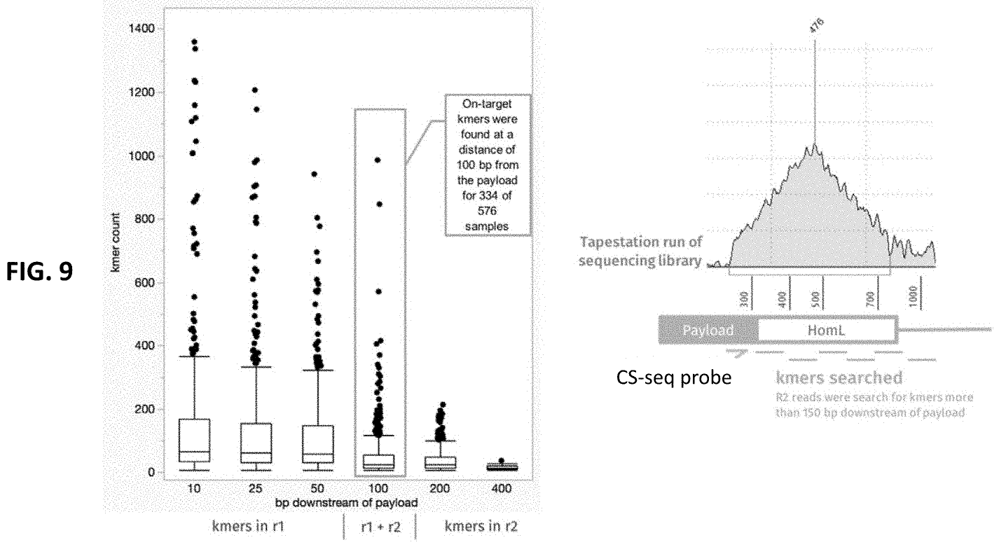

Data analysis: A k-mer taken from the payload was first used to determine which samples had any integration at all. K-mers were then designed beginning 10, 25, 50, 100, 200, and 400 bp downstream of the payload (all k-mers were 20 nucleotides) and corresponding to the expected downstream sequence for correct integrations. The R1 and R2 sequences were both searched for the 100 bp k-mers; proximal k-mers were searched in R1 reads only and distal k-mers were searched in R2 reads only because the R1 reads were expected to end ˜150 bp downstream of the payload. Data from an initial experiment is shown in FIG. 9.

### Conclusions

To detect an off-target integration using the method presented here, the sequencing library should ideally extend past the homology (hom) arm used for integration and into the surrounding genomic locus. Detection of “on-target” k-mers in that distal sequence would indicate a correct integration, while absence of the expected k-mer could indicate a possible ectopic integration or simply that no reads were generated in that region of the genome. Because the position of “downstream” tagmentation events is random, the number of samples for which k-mers can be reliably detected was expected to decrease as downstream distance increases.

In this example dataset, all samples had independently verified on-target integrations. On-target k-mers were detected at 100 bps downstream of the payload sequence (see FIG. 9) in about 60% of the samples. This means that if the homology arm length was less than 100 bp, the method described here would be able to indicate a possible off-target integration for about 60% of the samples. With the initial k-mer data in hand, alignment of the reads for samples where expected on-target k-mers were not found would allow determination of the site of ectopic integration, if any.

### Sequences of the Disclosure with Seq Id No Identifiers

| | | NUCLEIC ACID | |
|---|---|---|---|
| NAME | SOURCE | SEQ ID NO: | COMMENTS |
| CS-seq primer | Artificial | 1 | Table 1: CS-seq |
| PCR1-Fs | | | primer sequences |
| CS-seq primer | Artificial | 2 | Table 1: CS-seq |
| PCR2-F | | | primer sequences |
| CS-seq primer | Artificial | 3 | Table 1: CS-seq |
| PCR1-R | | | primer sequences |
| CS-seq primer | Artificial | 4 | Table 1: CS-seq |
| PCR2-R | | | primer sequences |
| Primer SG-seq | Artificial | 5 | Table 2: SG-seq |
| PCR1-F | | | primer sequences |
| Primer SG-seq | Artificial | 6 | Table 2: SG-seq |
| PCR2-F | | | primer sequences |
| Primer SG-seq | Artificial | 7 | Table 2: SG-seq |
| PCR1-R | | | primer sequences |
| Primer SG-seq | Artificial | 8 | Table 2: SG-seq |
| PCR1-R | | | primer sequences |
| Primer SG-seq | Artificial | 9 | Table 2: SG-seq |
| PCR1-R | | | primer sequences |
| Primer SG-seq | Artificial | 10 | Table 2: SG-seq |
| PCR1-R | | | primer sequences |
| Primer SG-seq | Artificial | 11 | Table 2: SG-seq |
| PCR1-R | | | primer sequences |
| Primer SG-seq | Artificial | 12 | Table 2: SG-seq |
| PCR1-R | | | primer sequences |
| Primer SG-seq | Artificial | 13 | Table 2: SG-seq |
| PCR2-R | | | primer sequences |

### Numbered Embodiments of the Disclosure

Other subject matter contemplated by the present disclosure is set out in the following numbered embodiments:

1. A method for identifying one or a plurality of genetic edits introduced into a microbial strain, the method comprising:
(a) appending an adaptor comprising a universal sequence to nucleic acid fragments from a plurality of nucleic acid fragments prepared from nucleic acid obtained from a microbial strain, wherein the microbial strain comprises one or a plurality of genetic edits previously introduced, wherein each genetic edit from the one or the plurality of genetic edits comprises a common sequence;
(b) amplifying each of the nucleic acid fragments from step (a) in a polymerase chain reaction (PCR) using a primer pair comprising a first primer comprising a sequence complementary to the common sequence at its 3′ end and a 5′ tail comprising non-complementary sequence and a second primer comprising sequence complementary to the universal sequence at its 3′ end and a 5′ tail comprising non-complementary sequence, optionally, wherein the non-complementary sequence of the first primer and the second primer each comprise sequencing primer binding sites; and
(c) performing molecular analysis on amplicons generated from the PCR performed in step (b), thereby identifying the one or the plurality of genetic edits in the microbial strain.
   2. A method for identifying one or a plurality of genetic edits introduced into a microbial strain, the method comprising:
(a) appending an adaptor comprising a universal sequence to nucleic acid fragments from a plurality of nucleic acid fragments prepared from nucleic acid obtained from a microbial strain, wherein the microbial strain comprises one or a plurality of genetic edits previously introduced, wherein each genetic edit from the one or the plurality of genetic edits comprises a common sequence;
(b) amplifying each of the nucleic acid fragments from step (a) in a first polymerase chain reaction (PCR) using a first primer pair comprising a first primer comprising a sequence complementary to the common sequence at its 3′ end and a 5′ tail comprising non-complementary sequence and a second primer comprising sequence complementary to the universal sequence at its 3′ end and a 5′ tail comprising non-complementary sequence;
(c) amplifying amplicons generated in step (b) in a second PCR using a second primer pair comprising a first primer comprising a 3′ end comprising sequence complementary to the non-complementary sequence in the 5′ tail of the first primer from the first primer pair and a second primer comprising a 3′ end comprising sequence complementary to the non-complementary sequence in the 5′ tail of the second primer from the first primer pair, wherein the first primer and the second primer from the second primer pair each comprise 5′ tails comprising non-complementary sequence, and optionally each of the 5′ tails of the second primer pair comprise sequencing primer binding sites; and
(d) performing molecular analysis on amplicons generated from the PCR performed in step (c), thereby identifying the one or the plurality of genetic edits in the microbial strain.
   3. A method for identifying one or a plurality of genetic edits introduced into a microbial strain, the method comprising:
(a) amplifying nucleic acid obtained from a microbial strain in a first polymerase chain reaction (PCR), wherein the microbial strain comprises one or a plurality of genetic edits, and wherein each genetic edit from the one or the plurality of genetic edits comprises a common sequence, wherein the first PCR utilizes a first primer pair comprising a first primer comprising a sequence complementary to the common sequence at its 3′ end and a 5′ tail comprising a first universal sequence and a plurality of second primers comprising a priming sequence complementary to a variable locus-specific sequence at its 3′ end and a 5′ tail comprising a second universal sequence that is common among all second primers;
(b) amplifying amplicons generated in step (a) in a second PCR using a second primer pair comprising a first primer comprising a 3′ end comprising sequence complementary to the first universal sequence in the 5′ tail of the first primer from the first primer pair and a second primer comprising a 3′ end comprising sequence complementary to the second universal sequence in the 5′ tail of each of the second primers from the first primer pair, wherein the first primer and the second primer from the second primer pair each comprise 5′ tails comprising non-complementary sequence, and optionally each of the 5′ tails of the second primer pair comprise sequencing primer binding sites; and
(c) performing molecular analysis on amplicons generated from the second PCR performed in step (b), thereby identifying the one or the plurality of genetic edits in the microbial strain.
   4. A method for identifying one or a plurality of genetic edits introduced into a microbial strain, the method comprising:
(a) amplifying nucleic acid obtained from a microbial strain in a polymerase chain reaction (PCR), wherein the microbial strain comprises one or a plurality of genetic edits, and wherein each genetic edit from the one or the plurality of genetic edits comprises a common sequence, wherein the PCR utilizes a primer pair comprising a first primer comprising a sequence complementary to the common sequence at its 3′ end and a 5′ tail comprising a first universal sequence and a plurality of second primers comprising a priming sequence complementary to a variable locus-specific sequence at its 3′ end and a 5′ tail comprising a second universal sequence that is common among all second primers, optionally, wherein the first primer and each second primer of the plurality of second primers each comprise sequencing primer binding sites in the 5′ tail; and
(b) performing molecular analysis on amplicons generated from the PCR performed in step (a), thereby identifying the one or the plurality of genetic edits in the microbial strain.
   5. The method of embodiment 1 or 2, wherein step (a) is performed in a transposon mediated adapter addition reaction.
   6. The method of embodiment 1, 2 or 5, wherein step (a) is performed in a tagmentation reaction.
   7. The method of embodiment 1 or 2, wherein step (a) is performed by fragmenting the nucleic acid derived from the microbial strain and ligating the adaptors comprising the universal sequence to the nucleic acid fragments.
   8. The method of embodiment 1 or 4, wherein the non-complementary sequence of the first primer and/or the second primer further comprise a sample specific index sequence.
   9. The method of embodiment 2 or 3, wherein the non-complementary sequence of the first primer and/or the second primer of the second primer pair further comprise a sample specific index sequence.
   10. The method of embodiment 3 or 4, wherein the priming sequence in the plurality of second primers comprises a mixture of fully or partially random nucleotides and nucleotides that are complementary to the variable locus-specific sequence.
   11. The method of any one of embodiments 3-4 or 10, wherein the priming sequence comprises at least 1, at least 2, at least 3, at least 4, at least 5, at least 6, at least 7, at least 8, at least 9 or at least 10 nucleotides that are complementary to the variable locus-specific sequence.
   12. The method of any one of embodiments 3-4 or 10, wherein the priming sequence comprises at least 3-5 nucleotides that are complementary to the variable locus-specific sequence.
   13. The method of any one of embodiments 3-4 or 10, wherein the priming sequence comprises 1, 2, 3, 4, 5, 6, 7, 8, 9 or 10 nucleotides that are complementary to the variable locus-specific sequence.
   14. The method of any one of embodiments 3-4 or 10, wherein the priming sequence comprises between 0-3, between 1-4, between 2-5, between 3-6, between 4-7, between 5-8, between 6-9, between 7-10 or between 8-11 nucleotides that are complementary to the variable locus-specific sequence.
   15. The method of any one of embodiments 3-4 or 10-14, wherein the variable locus-specific sequence is near the one or each genetic edit from the plurality of genetic edits.
   16. The method of any one of embodiments 3-4 or 10-15, wherein the variable locus-specific sequence is present in the microbial strain at least once near the one or each genetic edit of the plurality of genetic edits.
   17. The method of any one of embodiments 3-4 or 10-16, wherein the variable locus-specific sequence is less than 3 kilobases (kbs), less than 1.5 kbs, less than 1 kb, less than 750 base-pairs (bps), less than 500 bps, less than 250 bps, less than 125 bps, less than 100 bps, less than 75 bps, less than 50 bps, less than 25 bps, less than 20 bps, less than 15 bps, less than 10 bps, or less than 5 bps away from the one or each of the plurality of genetic edits.
   18. The method of any one of embodiments 3-4 or 10-17, wherein the variable locus-specific sequence is less than 1.5 kb away from the one or each of the plurality of genetic edits.
   19. The method of embodiment 1 or 3, wherein the molecular analysis comprises amplicon size selection on the amplicons generated from the PCR performed in step (b).
   20. The method of embodiment 2, wherein the molecular analysis comprises amplicon size selection on the amplicons generated from the PCR performed in step (c).
   21. The method of embodiment 4, wherein the molecular analysis comprises amplicon size selection on the amplicons generated from the PCR performed in step (a).
   22. The method of any one of embodiments 19-21, wherein the amplicon size selection comprises digestion and/or gel electrophoresis of the amplicons, optionally wherein the electrophoresis is preceded by the digestion.
   23. The method of any one of the above embodiments, wherein the molecular analysis comprises DNA sequencing.
   24. The method of any one of the above embodiments, wherein the molecular analysis of the amplicons comprises DNA sequencing using sequencing primers directed to the sequencing primer binding sites.
   25. The method of any one of the above embodiments, wherein the molecular analysis comprises first, second, or third generation DNA sequencing.
   26. The method of any one of the above embodiments, further comprising comparing sequence reads obtained from the sequencing of the amplicons to a reference database for the microbial strain using a computer-implemented method, thereby identifying the one or the plurality of genetic edits.
   27. The method of embodiment 26, wherein the computer-implemented method utilizes a sequence similarity search program, a sequence composition search program or a combination thereof.
   28. The method of embodiment 27, wherein the sequence similarity search program employs a basic local alignment search tool (BLAST) algorithm, fuzzy logic, lowest common ancestor (LCA) algorithm or a profile hidden Markov Model (pHMM).
   29. The method of embodiment 27, wherein the sequence composition search program employs interpolated Markov models (IMMs), naive Bayesian classifiers, k-mers or k-means/k-nearest-neighbor algorithms.
   30. The method of embodiment 29, wherein the sequence composition search program employs k-mers.
   31. The method of embodiment 30, wherein the k-mers comprise short nucleotide sequences comprising nucleotide bases complementary to a sequence near the one or each of the plurality of genetic edits, wherein detection of the short nucleotide sequence in the sequence reads indicates presence of the one or each of the plurality of genetic edits in the microbial strain.
   32. The method of embodiment 31, wherein the sequence near the one or each of the plurality of genetic edits is within 25 base pairs (bps), 20 bps, 15 bps, 10 bps, or 5 bps of the one or each of the plurality of genetic edits.
   33. The method of any one of the above embodiments, wherein the one or the plurality of genetic edits is in an episome, chromosome or other genomic DNA.
   34. The method of any one of the above embodiments, wherein the obtaining of the nucleic acid entails lysing the microbial strain.
   35. The method of any one of the above embodiments, wherein the obtaining of the nucleic acid entails isolating the nucleic acid from the microbial strain.
   36. The method of any one of the above embodiments, wherein the obtaining of the nucleic acid entails whole genome amplification (WGA) or multiple displacement amplification (MDA) of nucleic acid isolated from the microbial strain.
   37. The method of any one of embodiments 1-35, wherein the obtaining of the nucleic acid entails performing a boil preparation of the microbial strain.
   38. The method of embodiment 1, wherein the first primer is specific to a genetic edit and the second primer is specific to a single universal sequence found in each adapter.
   39. The method of embodiment 2, wherein the first primer of the second primer pair is specific to a genetic edit and the second primer of the second primer pair is specific to a single universal sequence found in each adapter.
   40. The method of any one of the above embodiments, wherein the genetic edits were introduced into the microbial strain by an iterative editing method, wherein the iterative method comprises:

(a) introducing into a microbial host cell a first plasmid comprising a first repair fragment and a selection marker gene, wherein the microbial host cell comprises a site-specific restriction enzyme or a sequence encoding a site-specific restriction enzyme is introduced into the microbial host cell along with the first plasmid, wherein the site-specific restriction enzyme targets a first locus in the microbial host cell, and wherein the first repair fragment comprises homology arms separated by a sequence for a genetic edit comprising a common sequence in or adjacent to a first locus in the microbial host cell, wherein the homology arms comprise sequence homologous to sequence that flanks the first locus in the microbial host cell;

(b) growing the microbial host cells from step (a) in a medium selective for microbial host cells expressing the selection marker gene and isolating microbial host cells from cultures derived therefrom;

(c) growing the microbial host cells isolated in step (b) in media not selective for the selection marker gene and isolating microbial host cells from cultures derived therefrom; and

(d) repeating steps (a)-(c) in one or more additional rounds in the microbial host cells isolated in step (c), wherein each of the one or more additional rounds comprises introducing an additional plasmid comprising an additional repair fragment, wherein the additional repair fragment comprises homology arms separated by a sequence for a genetic edit comprising a common sequence in or adjacent to a locus in the microbial host cell, wherein the homology arms comprise sequence homologous to sequence that flanks the locus in the microbial host cell, wherein the additional plasmid comprises a different selection marker gene than the selection marker gene introduced in a previous round of selection, and wherein the microbial host cell comprises a site-specific restriction enzyme or a sequence encoding a site-specific restriction enzyme is introduced into the microbial host cell along with the additional plasmid that targets the first locus or another locus in the microbial host cell, thereby iteratively editing the microbial host cell to generate the microbial strain comprising the plurality of genetic edits;

wherein a counterselection is not performed after at least one round of editing.
41. The method of any one of embodiments 1-39, wherein the genetic edits were introduced into the microbial strain by an iterative editing method, wherein the iterative method comprises:

(a) introducing into the microbial host cell a first plasmid, a first guide RNA (gRNA) and a first repair fragment, wherein the gRNA comprises a sequence complementary to a first locus in the microbial host cell, wherein the first repair fragment comprises homology arms separated by a sequence for a genetic edit comprising a common sequence in or adjacent to a first locus in the microbial host cell, wherein the homology arms comprise sequence homologous to sequence that flanks the first locus in the microbial host cell, wherein the first plasmid comprises a selection marker gene and at least one or both of the gRNA and the repair fragment, and wherein:
(i) the microbial host cell comprises an RNA-guided DNA endonuclease; or
(ii) an RNA-guided DNA endonuclease is introduced into the microbial host cell along with the first plasmid;

(b) growing the microbial host cells from step (a) in a media selective for microbial host cells expressing the selection marker gene and isolating microbial host cells from cultures derived therefrom;

(c) growing the microbial host cells isolated in step (b) in medium not selective for the selection marker gene and isolating microbial host cells from cultures derived therefrom; and

(d) repeating steps (a)-(c) in one or more additional rounds in the microbial host cells isolated in step (c), wherein each of the one or more additional rounds comprises introducing an additional plasmid, an additional gRNA and an additional repair fragment, wherein the additional gRNA comprises sequence complementary to a locus in the microbial host cell, wherein the additional repair fragment homology arms separated by a sequence for a genetic edit comprising a common sequence in or adjacent to a locus in the microbial host cell, wherein the homology arms comprise sequence homologous to sequence that flanks the locus in the microbial host cell, wherein the additional plasmid comprises a different selection marker gene than the selection marker gene introduced in a previous round of selection, and wherein the additional plasmid comprises at least one or both of the additional gRNA and the additional repair fragment, thereby iteratively editing the microbial host cell to generate the microbial strain comprising the plurality of genetic edits;

wherein a counterselection is not performed after at least one round of editing.
42. The method of any one of embodiments 1-39, wherein the genetic edits were introduced into the microbial strain by an iterative editing method, wherein the iterative method comprises:

(a) introducing into the microbial host cell a first plasmid comprising a first repair fragment and a selection marker gene, wherein the first repair fragment comprises homology arms separated by a sequence for a genetic edit comprising a common sequence in or adjacent to a first locus in the microbial host cell, wherein the homology arms comprise sequence homologous to sequence that flanks the first locus in the microbial host cell;

(b) growing the microbial host cells from step (a) in a medium selective for microbial host cells expressing the selection marker gene and isolating microbial host cells from cultures derived therefrom;

(c) growing the microbial host cells isolated in step (b) in media not selective for the selection marker gene and isolating microbial host cells from cultures derived therefrom; and

(d) repeating steps (a)-(c) in one or more additional rounds in the microbial host cells isolated in step (c), wherein each of the one or more additional rounds comprises introducing an additional plasmid comprising an additional repair fragment, wherein the additional repair fragment comprises homology arms separated by sequence for a genetic edit comprising a common sequence in or adjacent to a locus in the microbial host cell, wherein the homology arms comprise sequence homologous to sequence that flanks the locus in the microbial host cell, and wherein the additional plasmid comprises a different selection marker gene than the selection marker gene introduced in a previous round of selection, thereby iteratively editing the microbial host cell to generate the microbial strain comprising the plurality of genetic edits; wherein a counterselection is not performed after at least one round of editing.

43. The method of any one of embodiments 1-39, wherein the genetic edits were introduced into the microbial strain by a pooled editing method, wherein the pooled method comprises:

(a) combining a base population of microbial host cells with a first pool of editing plasmids, wherein each editing plasmid in the pool comprises at least one repair fragment, and wherein the pool of editing plasmids comprises at least two different repair fragments, wherein each editing plasmid in the pool further comprises a selection marker gene, and wherein each repair fragment comprises sequence for one or more genetic edits comprising a common sequence in or adjacent to one or more target loci in the microbial host cells, and wherein sequence for each of the one or more genetic edits lies between homology arms, wherein the homology arms comprise sequence homologous to sequence that flanks a target loci from the one or more target loci in the microbial host cells;

(b) introducing into individual microbial host cells from step (a) a plasmid or plasmids from the pool of editing plasmids; and

(c) growing the microbial host cells from step (b) in a medium selective for microbial host cells expressing the selection marker gene and isolating microbial host cells from cultures derived therefrom, thereby generating the microbial strain comprising the plurality of genetic edits.

44. The method of any one of embodiments 1-39, wherein the plurality of genetic edits were introduced into the microbial strain by a pooled editing method, wherein the pooled method comprises:

(a) combining a base population of microbial host cells with a first pool of editing plasmids, wherein each editing plasmid in the pool comprises at least one repair fragment, wherein the pool of editing plasmids comprises at least two different repair fragments, wherein each editing plasmid in the pool of editing plasmids further comprises a selection marker gene, and wherein the microbial host cells comprise one or more site-specific restriction enzymes or one or more sequences encoding one or more site-specific restriction enzymes is/are introduced into the microbial host cells along with the first pool of editing plasmids, wherein the one or more site-specific restriction enzymes target one or more target loci in the microbial host cells, wherein each repair fragment comprises sequence for one or more genetic edits comprising a common sequence in or adjacent to one or more target loci targeted by the one or more site-specific restriction enzymes, and wherein sequence for each of the one or more genetic edits lies between homology arms, wherein the homology arms comprise sequence homologous to sequence that flanks a target loci from the one or more target loci in the microbial host cell;

(b) introducing into individual microbial host cells from step (a) a plasmid or plasmids from the pool of editing plasmids; and

(c) growing the microbial host cells from step (b) in a medium selective for microbial host cells expressing the selection marker gene and isolating microbial host cells from cultures derived therefrom, thereby generating the microbial strain comprising the plurality of genetic edits.

45. The method of any one of embodiments 1-39, wherein the genetic edits were introduced into the microbial strain by a pooled editing method, wherein the pooled method comprises:

(a) combining a base population of microbial host cells with a first pool of editing constructs comprising one or more editing plasmids, wherein each editing plasmid in the first pool of editing constructs comprises a selection marker gene and one or both of a guide RNA (gRNA) and a repair fragment, wherein the microbial host cells comprise an RNA-guided DNA endonuclease or an RNA-guided DNA endonuclease is introduced into the microbial host cells along with the first pool of editing constructs, and wherein the first pool of editing constructs comprise:
(i) gRNAs that target the same target locus or loci, and at least two different repair fragments, wherein each repair fragment comprises a sequence for one or more genetic edits comprising a common sequence in or adjacent to the target locus, and wherein sequence for each of the genetic edits lies between homology arms, wherein the homology arms comprise sequence homologous to sequence that flanks the target locus in the microbial host cell;
(ii) gRNAs that target at least two different target loci, and at least two different repair fragments, wherein each repair fragment comprises a sequence for the same one or more genetic edits comprising a common sequence in or adjacent to the target loci, and wherein sequence for each of the genetic edits lies between homology arms, wherein the homology arms comprise sequence homologous to sequence that flanks the target loci in the microbial host cell; or
(iii) gRNAs that target at least two different target loci, and at least two different repair fragments, wherein each repair fragment comprises a sequence for one or more genetic edits comprising a common sequence in or adjacent to the target loci, and wherein sequence for each of the genetic edits lies between homology arms, wherein the homology arms comprise sequence homologous to sequence that flanks the target loci in the microbial host cell;

(b) introducing into individual microbial host cells from step (a) the first pool of editing constructs comprising the one or more editing plasmids, wherein the first pool of editing constructs comprise gRNAs and repair fragments according to any one of step (a)(i)-(iii); and

(c) growing the microbial host cells from step (b) in a medium selective for microbial host cells expressing the selection marker gene and isolating microbial host cells from cultures derived therefrom, thereby generating the microbial strain comprising the plurality of genetic edits.

46. The method of any one of embodiments 1-39, wherein the genetic edits were introduced into the microbial strain by a pooled editing method, wherein the pooled method comprises:

(a) combining a base population of microbial host cells with a first pool of editing constructs comprising one or more editing plasmids, wherein each editing plasmid in the first pool of editing constructs comprises a selection marker gene and one or both of a guide RNA (gRNA) and a repair fragment, and wherein the first pool of editing constructs comprise:
(i) gRNAs that target the same target locus or loci, and at least two different repair fragments, wherein each repair fragment comprises a sequence for one or more genetic edits comprising a common sequence in or adjacent to the target locus, and wherein sequence for each of the genetic edits lies between homology arms, wherein the homology arms comprise sequence homologous to sequence that flanks the target locus in the microbial host cell;
(ii) gRNAs that target at least two different target loci, and at least two different repair fragments, wherein each repair fragment comprises a sequence for the same one or more genetic edits comprising a common sequence in or adjacent to the target loci, and wherein sequence for each of the genetic edits lies between homology arms, wherein the homology arms comprise sequence homologous to sequence that flanks the target loci in the microbial host cell; or
(iii) gRNAs that target at least two different target loci, and at least two different repair fragments, wherein each repair fragment comprises a sequence for one or more genetic edits comprising a common sequence in or adjacent to the target loci, and wherein sequence for each of the genetic edits lies between homology arms, wherein the homology arms comprise sequence homologous to sequence that flanks the target loci in the microbial host cell;

(b) introducing into individual microbial host cells from step (a) an RNA-guided DNA endonuclease and the first pool of editing constructs comprising the one or more editing plasmids, wherein the first pool of editing constructs comprise gRNAs and repair fragments according to any one of step (a)(i)-(iii); and

(c) growing the microbial host cells from step (b) in a medium selective for microbial host cells expressing the selection marker gene and isolating microbial host cells from cultures derived therefrom, thereby generating the microbial strain comprising the plurality of genetic edits.

47. The method of any one of the above embodiments, wherein the common sequence in at least one genetic edit in the plurality of genetic edits is different from the common sequence in each other genetic edit in the plurality of genetic edits.
48. The method of any one of the above embodiments, wherein the common sequence in each genetic edit in the plurality of genetic edits is different from the common sequence in each other genetic edit in the plurality of genetic edits.
49. The method of any one of the above embodiments, wherein the common sequence is selected from any genetic element including a promoter sequence, a termination sequence, a degron sequence, a protein solubility tag sequence, a protein degradation tag sequence, a ribosomal binding site (RBS) sequence, a landing pad primer binding sequence, an antibiotic resistance gene sequence or any portion thereof.
50. The method of any one of the above embodiments, wherein the common sequence is specific to a genetic edit.
51. The method of embodiment 33, wherein the chromosome is from bacteria or fungi.

The various embodiments described above can be combined to provide further embodiments. All of the U.S. patents, U.S. patent application publications, U.S. patent application, foreign patents, foreign patent application and non-patent publications referred to in this specification and/or listed in the Application Data Sheet are incorporated herein by reference, in their entirety. Aspects of the embodiments can be modified, if necessary to employ concepts of the various patents, application and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

1. A method for identifying one or a plurality of genetic edits introduced into a microbial strain, the method comprising:
(a) appending an adaptor comprising a universal sequence to nucleic acid fragments from a plurality of nucleic acid fragments prepared from nucleic acid obtained from a microbial strain, wherein the microbial strain comprises one or a plurality of genetic edits previously introduced, wherein each genetic edit from the one or the plurality of genetic edits comprises a common sequence;
(b) amplifying each of the nucleic acid fragments from step (a) in a polymerase chain reaction (PCR) using a primer pair comprising a first primer comprising a sequence complementary to the common sequence at its 3′ end and a 5′ tail comprising non-complementary sequence and a second primer comprising sequence complementary to the universal sequence at its 3′ end and a 5′ tail comprising non-complementary sequence, optionally, wherein the non-complementary sequence of the first primer and the second primer each comprise sequencing primer binding sites; and
(c) performing molecular analysis on amplicons generated from the PCR performed in the preceding step, thereby identifying the one or the plurality of genetic edits in the microbial strain.

2.-3. (canceled)

4. A method for identifying one or a plurality of genetic edits introduced into a microbial strain, the method comprising:
(a) amplifying nucleic acid obtained from a microbial strain in a polymerase chain reaction (PCR), wherein the microbial strain comprises one or a plurality of genetic edits, and wherein each genetic edit from the one or the plurality of genetic edits comprises a common sequence, wherein the PCR utilizes a primer pair comprising a first primer comprising a sequence complementary to the common sequence at its 3′ end and a 5′ tail comprising a first universal sequence and a plurality of second primers comprising a priming sequence complementary to a variable locus-specific sequence at its 3′ end and a 5′ tail comprising a second universal sequence that is common among all second primers, optionally, wherein the first primer and each second primer of the plurality of second primers each comprise sequencing primer binding sites in the 5′ tail; and
(b) performing molecular analysis on amplicons generated from the PCR performed in the preceding step, thereby identifying the one or the plurality of genetic edits in the microbial strain.

5. The method of claim 1, wherein step (a) is performed in a transposon mediated adapter addition reaction or by fragmenting the nucleic acid derived from the microbial strain and ligating the adaptors comprising the universal sequence to the nucleic acid fragments.

6.-9. (canceled)

10. The method of claim 4, wherein the priming sequence in the plurality of second primers comprises a mixture of fully or partially random nucleotides and at least 1, at least 2, at least 3, at least 4, at least 5, at least 6, at least 7, at least 8, at least 9 or at least 10 nucleotides that are complementary to the variable locus-specific sequence nucleotides that are complementary to the variable locus-specific sequence.

11.-16. (canceled)

17. The method of claim 4, wherein the variable locus-specific sequence is less than 3 kilobases (kbs), less than 1.5 kbs, less than 1 kb, less than 750 base-pairs (bps), less than 500 bps, less than 250 bps, less than 125 bps, less than 100 bps, less than 75 bps, less than 50 bps, less than 25 bps, less than 20 bps, less than 15 bps, less than 10 bps, or less than 5 bps away from the one or each of the plurality of genetic edits.

18. (canceled)

19. The method of claim 1, wherein the molecular analysis comprises amplicon size selection on the amplicons generated from the PCR performed in step (b) or DNA sequencing.

20. (canceled)

21. The method of claim 4, wherein the molecular analysis comprises amplicon size selection on the amplicons generated from the PCR performed in step (a) or DNA sequencing.

22.-26. (canceled)

27. The method of claim 1, further comprising comparing sequence reads obtained from the sequencing of the amplicons to a reference database for the microbial strain using a computer-implemented method that utilizes a sequence similarity search program, a sequence composition search program or a combination thereof, thereby identifying the one or the plurality of genetic edits.

28.-30. (canceled)

31. The method of claim 27, wherein the sequence composition search program employs k-mers, wherein the k-mers comprise short nucleotide sequences comprising nucleotide bases complementary to a sequence within 25 base pairs (bps), 20 bps, 15 bps, 10 bps, or 5 bps of the one or each of the plurality of genetic edits of the one or each of the plurality of genetic edits, wherein detection of the short nucleotide sequence in the sequence reads indicates presence of the one or each of the plurality of genetic edits in the microbial strain.

32.-47. (canceled)

48. The method of claim 1, wherein the common sequence in at least one genetic edit in the plurality of genetic edits is different from the common sequence in each other genetic edit in the plurality of genetic edits.

49. (canceled)

50. The method of claim 1, wherein the common sequence is selected from any genetic element including a promoter sequence, a termination sequence, a degron sequence, a protein solubility tag sequence, a protein degradation tag sequence, a ribosomal binding site (RBS) sequence, a landing pad primer binding sequence, an antibiotic resistance gene sequence or any portion thereof.

51. The method of claim 1, wherein the common sequence is specific to a genetic edit.

52. (canceled)

53. The method of claim 1, further comprising amplifying amplicons generated in step (b) in a second PCR prior to step (c), wherein the second PCR uses a second primer pair comprising a first primer comprising a 3′ end comprising sequence complementary to the non-complementary sequence in the 5′ tail of the first primer from the first primer pair and a second primer comprising a 3′ end comprising sequence complementary to the non-complementary sequence in the 5′ tail of the second primer from the first primer pair, wherein the first primer and the second primer from the second primer pair each comprise 5′ tails comprising non-complementary sequence, and optionally each of the 5′ tails of the second primer pair comprise sequencing primer binding sites.

54. The method of claim 4, further comprising amplifying amplicons generated in step (a) in a second PCR prior to step (b), wherein the second PCR uses a second primer pair comprising a first primer comprising a 3′ end comprising sequence complementary to the first universal sequence in the 5′ tail of the first primer from the first primer pair and a second primer comprising a 3′ end comprising sequence complementary to the second universal sequence in the 5′ tail of each of the second primers from the first primer pair, wherein the first primer and the second primer from the second primer pair each comprise 5′ tails comprising non-complementary sequence, and optionally each of the 5′ tails of the second primer pair comprise sequencing primer binding sites.

55. The method of claim 4, further comprising comparing sequence reads obtained from the sequencing of the amplicons to a reference database for the microbial strain using a computer-implemented method that utilizes a sequence similarity search program, a sequence composition search program or a combination thereof, thereby identifying the one or the plurality of genetic edits.

56. The method of claim 55, wherein the sequence composition search program employs k-mers, wherein the k-mers comprise short nucleotide sequences comprising nucleotide bases complementary to a sequence within 25 base pairs (bps), 20 bps, 15 bps, 10 bps, or 5 bps of the one or each of the plurality of genetic edits of the one or each of the plurality of genetic edits, wherein detection of the short nucleotide sequence in the sequence reads indicates presence of the one or each of the plurality of genetic edits in the microbial strain.

57. The method of claim 4, wherein the common sequence in at least one genetic edit in the plurality of genetic edits is different from the common sequence in each other genetic edit in the plurality of genetic edits.

58. The method of claim 4, wherein the common sequence is selected from any genetic element including a promoter sequence, a termination sequence, a degron sequence, a protein solubility tag sequence, a protein degradation tag sequence, a ribosomal binding site (RBS) sequence, a landing pad primer binding sequence, an antibiotic resistance gene sequence or any portion thereof.

59. The method of claim 4, wherein the common sequence is specific to a genetic edit.
